SEQUENCE LISTING (92 sequences)

SEQ ID NO: 1 - protein, 8 aa, synthetic construct
  REGION 1..8: note = CDR1 of VHH1
GRLSSSNS

SEQ ID NO: 2 - protein, 7 aa, synthetic construct
  REGION 1..7: note = CDR2 of VHH1
ITGGGET

SEQ ID NO: 3 - protein, 9 aa, synthetic construct
  REGION 1..9: note = CDR3 of VHH1
NFWPPLINY

SEQ ID NO: 4 - protein, 115 aa, synthetic construct
  REGION 1..115: note = VHH1
DVQLVESGGGLVQPGGSLRLSCVASGRLSSSNSMAWYRQVPGKRRELVAGITGGGETNYADFVGGRFTISRDNAKNGLYLQLNGLKPEDTAAYYCNFWPPLINYWGQGTQVTVSS

SEQ ID NO: 5 - protein, 760 aa, Homo sapiens
  REGION 1..760: note = human fibroblast activation protein alpha (FAP), transcript variant 1
MKTWVKIVFGVATSAVLALLVMCIVLRPSRVHNSEENTMRALTLKDILNGTFSYKTFFPNWISGQEYLHQSADNNIVLYNIETGQSYTILSNRTMKSVNASNYGLSPDRQFVYLESDYSKLWRYSYTATYYIYDLSNGEFVRGNELPRPIQYLCWSPVGSKLAYVYQNNIYLKQRPGDPPFQITFNGRENKIFNGIPDWVYEEEMLATKYALWWSPNGKFLAYAEFNDTDIPVIAYSYYGDEQYPRTINIPYPKAGAKNPVVRIFIIDTTYPAYVGPQEVPVPAMIASSDYYFSWLTWVTDERVCLQWLKRVQNVSVLSICDFREDWQTWDCPKTQEHIEESRTGWAGGFFVSTPVFSYDAISYYKIFSDKDGYKHIHYIKDTVENAIQITSGKWEAINIFRVTQDSLFYSSNEFEEYPGRRNIYRISIGSYPPSKKCVTCHLRKERCQYYTASFSDYAKYYALVCYGPGIPISTLHDGRTDQEIKILEENKELENALKNIQLPKEEIKKLEVDEITLWYKMILPPQFDRSKKYPLLIQVYGGPCSQSVRSVFAVNWISYLASKEGMVIALVDGRGTAFQGDKLLYAVYRKLGVYEVEDQITAVRKFIEMGFIDEKRIAIWGWSYGGYVSSLALASGTGLFKCGIAVAPVSSWEYYASVYTERFMGLPTKDDNLEHYKNSTVMARAEYFRNVDYLLIHGTADDNVHFQNSAQIAKALVNAQVDFQAMWYSDQNHGLSGLSTNHLYTHMTHFLKQCFSLSD

SEQ ID NO: 6 - protein, 761 aa, Mus musculus
  REGION 1..761: note = murine fibroblast activation protein alpha (FAP)
MKTWLKTVFGVTTLAALALVVICIVLRPSRVYKPEGNTKRALTLKDILNGTFSYKTYFPNWISEQEYLHQSEDDNIVFYNIETRESYIILSNSTMKSVNATDYGLSPDRQFVYLESDYSKLWRYSYTATYYIYDLQNGEFVRGYELPRPIQYLCWSPVGSKLAYVYQNNIYLKQRPGDPPFQITYTGRENRIFNGIPDWVYEEEMLATKYALWWSPDGKFLAYVEFNDSDIPIIAYSYYGDGQYPRTINIPYPKAGAKNPVVRVFIVDTTYPHHVGPMEVPVPEMIASSDYYFSWLTWVSSERVCLQWLKRVQNVSVLSICDFREDWHAWECPKNQEHVEESRTGWAGGFFVSTPAFSQDATSYYKIFSDKDGYKHIHYIKDTVENAIQITSGKWEAIYIFRVTQDSLFYSSNEFEGYPGRRNIYRISIGNSPPSKKCVTCHLRKERCQYYTASFSYKAKYYALVCYGPGLPISTLHDGRTDQEIQVLEENKELENSLRNIQLPKVEIKKLKDGGLTFWYKMILPPQFDRSKKYPLLIQVYGGPCSQSVKSVFAVNWITYLASKEGIVIALVDGRGTAFQGDKFLHAVYRKLGVYEVEDQLTAVRKFIEMGFIDEERIAIWGWSYGGYVSSLALASGTGLFKCGIAVAPVSSWEYYASIYSERFMGLPTKDDNLEHYKNSTVMARAEYFRNVDYLLIHGTADDNVHFQNSAQIAKALVNAQVDFQAMWYSDQNHGISSGRSQNHLYTHMTHFLKQCFSLSD

SEQ ID NO: 7 - protein, 25 aa, synthetic construct
  REGION 1..25: note = FR1 of VHH1
DVQLVESGGGLVQPGGSLRLSCVAS

SEQ ID NO: 8 - protein, 17 aa, synthetic construct
  REGION 1..17: note = FR2 of VHH1
MAWYRQVPGKRRELVAG

SEQ ID NO: 9 - protein, 38 aa, synthetic construct
  REGION 1..38: note = FR3 of VHH1
NYADFVGGRFTISRDNAKNGLYLQLNGLKPEDTAAYYC

SEQ ID NO: 10 - protein, 11 aa, synthetic construct
  REGION 1..11: note = FR4 of VHH1
WGQGTQVTVSS

SEQ ID NO: 11 - protein, 9 aa, synthetic construct
  REGION 1..9: note = Exemplary HA tag
YPYDVPDYA

SEQ ID NO: 12 - protein, 10 aa, synthetic construct
  REGION 1..10: note = Exemplary HA tag
YPYDVPDYGS

SEQ ID NO: 13 - protein, 15 aa, synthetic construct
  REGION 1..15: note = Exemplary cysteine tag
SPSTPPTPSPSTPPC

SEQ ID NO: 14 - protein, 257 aa, Homo sapiens
  REGION 1..257: note = Human FOLR1
MAQRMTTQLLLLLVWVAVVGEAQTRIAWARTELLNVCMNAKHHKEKPGPEDKLHEQCRPWRKNACCSTNTSQEAHKDVSYLYRFNWNHCGEMAPACKRHFIQDTCLYECSPNLGPWIQQVDQSWRKERVLNVPLCKEDCEQWWEDCRTSYTCKSNWHKGWNWTSGFNKCAVGAACQPFHFYFPTPTVLCNEIWTHSYKVSNYSRGSGRCIQMWFDPAQGNPNEEVARFYAAAMSGAGPWAAWPFLLSLALMLLWLLS

SEQ ID NO: 15 - protein, 78 aa, synthetic construct
  REGION 1..78: note = IMGT: part of VHH2: N37-W118 = longest region of VHH2 interacting with human FOLR1
NHMSWYRQAPGKQRELAAIITSDGNTNYPDSVKGRFTISRDNAKNTVYLQMNSLKPEDTAVYYCNTFGRSVAGFYDYW

SEQ ID NO: 16 - protein, 76 aa, synthetic construct
  REGION 1..76: note = IMGT: part of VHH3: D38-W118 = longest region of VHH3 interacting with human FOLR1
DLAWYRRAPGKQRELAAIITSGGSTNYPDSVKGRFTISRDNAKNTVYLEMNSLKPEDTAVYYCNALRPWPQFSDYW

SEQ ID NO: 17 - protein, 30 aa, synthetic construct
  REGION 1..30: note = IMGT: 1st of 2 discrete regions of VHH2 interacting with human FOLR1 = N37-D69
NHMSWYRQAPGKQRELAAIITSDGNTNYPD

SEQ ID NO: 18 - protein, 14 aa, synthetic construct
  REGION 1..14: note = IMGT: 2nd of 2 discrete regions of VHH2 interacting with human FOLR1 = N105-W118
NTFGRSVAGFYDYW

SEQ ID NO: 19 - protein, 29 aa, synthetic construct
  REGION 1..29: note = IMGT: 1ST of 2 discrete regions of VHH3 interacting with human FOLR1 = D38-D69
DLAWYRRAPGKQRELAAIITSGGSTNYPD

SEQ ID NO: 20 - protein, 13 aa, synthetic construct
  REGION 1..13: note = IMGT: 2nd of 2 discrete regions of VHH3 interacting with human FOLR1 = N105-W118
NALRPWPQFSDYW

SEQ ID NO: 21 - protein, 119 aa, synthetic construct
  REGION 1..119: note = Full length sequence of VHH2
DVQLVESGGGLVQPGGSLRLSCAASESIFSRNHMSWYRQAPGKQRELAAIITSDGNTNYPDSVKGRFTISRDNAKNTVYLQMNSLKPEDTAVYYCNTFGRSVAGFYDYWGQGTQVTVSS

SEQ ID NO: 22 - protein, 118 aa, synthetic construct
  REGION 1..118: note = Full length sequence of VHH3
DVQLVESGGGLVQAGGSLRLSCAADEMKFSNYDLAWYRRAPGKQRELAAIITSGGSTNYPDSVKGRFTISRDNAKNTVYLEMNSLKPEDTAVYYCNALRPWPQFSDYWGQGTQVTVSS

SEQ ID NO: 23 - protein, 8 aa, synthetic construct
  REGION 1..8: note = CDR1 of VHH2
ESIFSRNH

SEQ ID NO: 24 - protein, 7 aa, synthetic construct
  REGION 1..7: note = CDR2 of VHH2
ITSDGNT

SEQ ID NO: 25 - protein, 13 aa, synthetic construct
  REGION 1..13: note = CDR3 of VHH2
NTFGRSVAGFYDY

SEQ ID NO: 26 - protein, 25 aa, synthetic construct
  REGION 1..25: note = FR1 of VHH2
DVQLVESGGGLVQPGGSLRLSCAAS

SEQ ID NO: 27 - protein, 17 aa, synthetic construct
  REGION 1..17: note = FR2 of VHH2
MSWYRQAPGKQRELAAI

SEQ ID NO: 28 - protein, 38 aa, synthetic construct
  REGION 1..38: note = FR3 of VHH2
NYPDSVKGRFTISRDNAKNTVYLQMNSLKPEDTAVYYC

SEQ ID NO: 29 - protein, 11 aa, synthetic construct
  REGION 1..11: note = FR4 of VHH2
WGQGTQVTVSS

SEQ ID NO: 30 - protein, 8 aa, synthetic construct
  REGION 1..8: note = CDR1 of VHH3
EMKFSNYD

SEQ ID NO: 31 - protein, 7 aa, synthetic construct
  REGION 1..7: note = CDR2 of VHH3
ITSGGST

SEQ ID NO: 32 - protein, 12 aa, synthetic construct
  REGION 1..12: note = CDR3 of VHH3
NALRPWPQFSDY

SEQ ID NO: 33 - protein, 25 aa, synthetic construct
  REGION 1..25: note = FR1 of VHH3
DVQLVESGGGLVQAGGSLRLSCAAD

SEQ ID NO: 34 - protein, 17 aa, synthetic construct
  REGION 1..17: note = FR2 of VHH3
LAWYRRAPGKQRELAAI

SEQ ID NO: 35 - protein, 38 aa, synthetic construct
  REGION 1..38: note = FR3 of VHH3
NYPDSVKGRFTISRDNAKNTVYLEMNSLKPEDTAVYYC

SEQ ID NO: 36 - protein, 11 aa, synthetic construct
  REGION 1..11: note = FR4 of VHH3
WGQGTQVTVSS

SEQ ID NO: 37 - protein, 9 aa, synthetic construct
  REGION 1..9: note = hemaglutinin tag (HA-tag)
YPYDVPDYA

SEQ ID NO: 38 - protein, 10 aa, synthetic construct
  REGION 1..10: note = tag
YPYDVPDYGS

SEQ ID NO: 39 - protein, 15 aa, synthetic construct
  REGION 1..15: note = non-limiting example of a cysteine tag
SPSTPPTPSPSTPPC

SEQ ID NO: 40 - protein, 8 aa, synthetic construct
  REGION 1..8: note = CDR1 sequence of VHH 2Rs15d
GYIFNSCG

SEQ ID NO: 41 - protein, 7 aa, synthetic construct
  REGION 1..7: note = CDR2 sequence of VHH 2Rs15d
ISGDGDT

SEQ ID NO: 42 - protein, 9 aa, synthetic construct
  REGION 1..9: note = CDR3 sequence of VHH 2Rs15d
AVCYNLETY

SEQ ID NO: 43 - protein, 8 aa, synthetic construct
  REGION 1..8: note = CDR1 sequence of VHH 2RVHH17c
GFIFSNDA

SEQ ID NO: 44 - protein, 8 aa, synthetic construct
  REGION 1..8: note = CDR2 sequence of VHH 2RVHH17c
INWSGTHT

SEQ ID NO: 45 - protein, 10 aa, synthetic construct
  REGION 1..10: note = CDR3 sequence of VHH 2RVHH17c
VTGYGVTKTP

SEQ ID NO: 46 - protein, 115 aa, synthetic construct
  REGION 1..115: note = VHH sequence 2Rs15d
QVQLQESGGGSVQAGGSLKLTCAASGYIFNSCGMGWYRQSPGRERELVSRISGDGDTWHKESVKGRFTISQDNVKKTLYLQMNSLKPEDTAVYFCAVCYNLETYWGQGTQVTVSS

SEQ ID NO: 47 - protein, 117 aa, synthetic construct
  REGION 1..117: note = VHH sequence 2RVHH17c
QVQLQESGGGLVQPGGSLRLSCAASGFIFSNDAMTWVRQAPGKGLEWVSSINWSGTHTNYADSVKGRFTISRDNAKRTLYLQMNSLKDEDTALYYCVTGYGVTKTPTGQGTQVTVSS

SEQ ID NO: 48 - protein, 123 aa, Camelus bactrianus
QVQLQESGGGSVQAGETLRLSCTASGFTFDDSDMGWYRQAPGNECELVSTISSDGSTYYADSVKGRFTISQDNAKNTVYLQMNSLKPEDTAVYYCAAHPLHYELGTCAALDYWGRGTQVTVSS

SEQ ID NO: 49 - protein, 123 aa, Camelus bactrianus
QVQLQESGGGSVQAGETLRLSCTASGFTFDDSDMGWYRQAPGNECELVSTISSDGSTYYADSVKGRFTISQDNAKNAVYLQMNSLKPEDTAVYYCAAHPLHYELGTCAALDYWGRGTQVTVSS

SEQ ID NO: 50 - protein, 123 aa, Camelus bactrianus
QVQLQESGGGSVQAGGSLRLSCTASGFTFDDSDVRWYRQAPGRECKLVSSISSDRSAYYEDSVKGRFTISQDNAKNTVYLQMNSLKPEDTAVYYCAAHPLHYELGTCAALDYWGRGTQVTVSS

SEQ ID NO: 51 - protein, 123 aa, Camelus bactrianus
QVQLQESGGGLVQPGGSLRLSCTASRFTFDDSDMAWYRQAPGNECELVSIISSDGSTYYADSVKGRFTISLDNTKSTVYLQMNSLKPEDTAVYYCAAHPLHYELGTCAALDYWGRGTQVTVSS

SEQ ID NO: 52 - protein, 118 aa, Camelus bactrianus
QVQLQESGGGSVQAGGSLRLSCTVSGFSFDDSDMGWYRRAPGNECELVSGISRDGSTYYADSVKGRFTISQDNAKNWVYLQMNSLKPEDTAVYYCAAATYSDYVCDYWTQGTQVTVSS

SEQ ID NO: 53 - protein, 125 aa, Camelus bactrianus
QVQLQESGGGSVQAGETLRLSCTASGFTFDDSDMGWYRQAPGNECELVSHISSDGSTYYADSVKGRFTISQDNAKNTVFLQMNSLKPEDTAVYYCAADKDARGYELGTCESLDYWGRGTQVTVSS

SEQ ID NO: 54 - protein, 125 aa, Camelus bactrianus
QVQLQESGGGSVQAGETLRLSCTASGFTFDDSDMGWYRQAPGNECELVSTISSDGSTYYADSVKGRFTISQDNAKNTVYLQMNSLKPEDTAVYYCAADKDARGYELGTCESLDYWGRGTQVTVSS

SEQ ID NO: 55 - protein, 123 aa, Camelus bactrianus
QVQLQESGGGSVQAGETLRLSCTASGFTFDESVMGWYRQAPGNECELVSTISSDGSTYYSNSVKGRFTISQDNAKNTVYLQMNSLKPEDTAVYYCAADDHTYELGTCEALNYWGRGTQVTVSS

SEQ ID NO: 56 - protein, 123 aa, Camelus bactrianus
QVQLQESGGGSVQAGETLRLSCTASGFTFDDSDMGWYRQAPGSECELVSTISSDGNTYYSNSVKGRFTISQDNAKNTVYLQMNSLKPEDTAVYYCAADDQHYELGTCEALDYWGRGTQVTVSS

SEQ ID NO: 57 - protein, 123 aa, Camelus bactrianus
QVQLQESGGGSVQAGETLKLSCTASGFTFDDSTMAWYRQAPGNECKLVSTISSDGSTYYADSVKGRFTISQDNAKNTVYLQMNSLKPEDTAVYYCAADDQNYELGTCEALDYWGRGTQVTVSS

SEQ ID NO: 58 - protein, 123 aa, Camelus bactrianus
QVQLQESGGGSVQAGETLRLSCTASGFTFDDSDMGWYRQAPGNECELVSHISSGGSTYYADSVKGRFTISQDNAKNTVYLQMNSLKPEDTAVYYCAADGSNYELGTCAALDYWGRGTQVTVSS

SEQ ID NO: 59 - protein, 123 aa, Camelus bactrianus
QVQLQESGGGSVQAGETLRLSCTASGFTFDDSDMGWYRQAPGNECELVSTISSDGSTYYADSVKGRFTISQDNAKNTVYLQMNSLKPEDTAVYYCAADGHKYELGTCAALDYWGRGTQVTVSS

SEQ ID NO: 60 - protein, 123 aa, Camelus bactrianus
QVQLQESGGGSVQAGETLRLSCTASGFTFDDSDMGWYRQAPGNECELVSTISADGSTFYADSVKGRFTISQDNAKNTVYLQMNSLKPEDTAVYYCASPENEYELGTCEALDYWGQGTQVTVSS

SEQ ID NO: 61 - protein, 123 aa, Camelus bactrianus
QVQLQESGGGSVQAGETLRLSCTASGFTFDDSDMGWYRQAPGNECELVSRISSDGSTYYADSVKGRFTISQDNAKNTVYLQMNSLKPEDTAVYYCAALEWEYELGTCEALDYWGQGTQVTVSS

SEQ ID NO: 62 - protein, 123 aa, Camelus bactrianus
QVQLQESGGGSVQAGGSLRLSCTASRFTFDDSDMGWYRQAPGNECELVSTISSDGATYYANSVKGRFTISQDNAANTVYLQMNSLKPEDTAVYYCAALEWEYELGTCEALDYWGQGTQVTVSS

SEQ ID NO: 63 - protein, 123 aa, Camelus bactrianus
QVQLQESGGGSVQAGGSLRLSCTASGFTFDDSDMVWYRQAPGNECELVSRISSDGSTYYADSVKGRFTISQDNAKNTVYLQMNSLKPEDTAVYYCAALEWEYELGTCEALDYWGQGTQVTVSS

SEQ ID NO: 64 - protein, 123 aa, Camelus bactrianus
QVQLQESGGGSVQAGGSLRLSCTASGFTFDDSDMGWYRQAPGNECELVSTISSDGSTYYADSVKGRFTISQDNAKNTVYLQMNSLKPEDTAVYYCADVQIPYGLGTCESLDYWGRGTQVTVSS

SEQ ID NO: 65 - protein, 122 aa, Camelus bactrianus
QVQLQESGGGSVQAGQTLRLSCTASGFTFDDSDMAWYRQAPGNECELVSKMRSDGSTYYADSVKGRFTISQDNAKNTVYLQMNSLKPEDTAVYYCAEDLPYGLGTCTSLDYWGRGTQVTVSS

SEQ ID NO: 66 - protein, 125 aa, Camelus bactrianus
QVQLQESGGGSVQAGGSLRLSCTASGFTFDDSDSDMGWYRQAPGNECELVSSISSDGSTYYADSVKGRFTISQDNAKNTVYLQMNSLKPEDTAVYYCAAINSGYELGTCESLDYWGRGTQVTVSS

SEQ ID NO: 67 - protein, 123 aa, Camelus bactrianus
QVQLQESGGGSVQAGGSLRLSCTASGFTFDDSVMGWFRKAPGNECELVSTISSDGSTYYADSVKGRFTISQDNAKNTVYLQMNNLKPEDTAVYYCAAINSGYELGTCESLDYWGRGTQVTVSS

SEQ ID NO: 68 - protein, 125 aa, Camelus bactrianus
QVQLQESGGGLVQPRGSLRLSCTASGFTFDDSDSDMGWYRQAPGNECELVSSISSDGSTYYADSVKGRFTISQDNAKNTVYLQMNSLKPEDTAVYYCAAINSGYELGTCESLDYWGRGTQVTVSS

SEQ ID NO: 69 - protein, 123 aa, Camelus bactrianus
QVQLQESGGGSVQAGGSLRLSCTASGFTFDDSDMGWYRQAPGNECELVSRISRDGTTYYADSVKGRFTISQDNAKNTVYLQMNSLKPEDTAVYYCADINSGYELGTCESLDYWGRGTQVTVSS

SEQ ID NO: 70 - protein, 123 aa, Camelus bactrianus
QVQLQESGGGSVQAGGSLKLSCSASGFTFDDTDMGWYRQAPGNECELVSTISSDGTTYYTDSVKGRFTISQDNAKNTVYLQMNSLKPEDTAVYYCAAINSGYELGTCESLDYWGRGTQVTVSS

SEQ ID NO: 71 - protein, 125 aa, Camelus bactrianus
QVQLQESGGGSVQAGETLRLSCTASGFTFDDSDSDMGWYRQAPGNECELVSSISSDGSTYYADSVKGRFTISQDNAKNTVYLQMNSLKPEDTAVYYCAAINSGYELGTCESLDYWGRGTQVTVSS

SEQ ID NO: 72 - protein, 125 aa, Camelus bactrianus
QVQLQESGGGSVQAGETLRLSCTASGFTFDDSDSDMGWYRQAPGNECELVSSISSDGSTYYADSVKGRFTISQDNAKNTVYLQMNSPKPEDTAVYYCAAINSGYELGTCESLDYWGRGTQVTVSS

SEQ ID NO: 73 - protein, 123 aa, Camelus bactrianus
QVQLQESGGGSVQAGETLRLSCTASGFTFDDSDMGWYRQAPGNECELVSSISSDGSTYYADSVKGRFTISQDNAKNTVYLQMNSLKPEDTGVYYCAAEGHRYELGTCAALDYWGRGTQVTVSS

SEQ ID NO: 74 - protein, 124 aa, Camelus bactrianus
QVQLQESGGGSVQAGETLRLSCTASGFTFDDSDMGWYRQAPGNECELVSTISSDGSTYYADSVKGRFTISQDNAKNTVYLQMNSLKPEDTAVYYCAADHGGGYELGTCAALDYWGRGTQVTVSS

SEQ ID NO: 75 - protein, 123 aa, Camelus bactrianus
QVQLQESGGGLVQPGGSLRLSCAASGFTFGDSGMGWYRQAPGNECELVSSVSSDGSTYYADSVKGRFTISQDNAKNTVYLRMNSLKPEDTAVYYCAADDHKYELGTCEALDYWGRGTQVTVSS

SEQ ID NO: 76 - protein, 123 aa, Camelus bactrianus
QVQLQESGGGSVQAGGSLRLSCTASGFTFDDLDMRWYRQAPGNECELVSIINSDGRTYYADSVKGRFAISQNNAKNTVYLQMNSLKPEDTAVYYCAADQHRYGLGTCEALDYWGRGTQVTVSS

SEQ ID NO: 77 - protein, 123 aa, Camelus bactrianus
QVQLQESGGGSVQAGETLRLSCTASGFTFDDSDMGWYRQAPGNECELVSTISSDGRTYYADSVKGRFAISQNNAKNTVYLQMNSLKPEDTAVYYCAADQHRYGLGTCEALDYWGRGTQVTVSS

SEQ ID NO: 78 - protein, 122 aa, Camelus bactrianus
QVQLQESGGGSVQAGETLRLSCTASGFTFNDSNMGWYRQAPGHECELVSTISSDGSTYYADSVKGRFTISQNNARNTVYLQMNSLKPEDTAVYYCAGDWGYELGICTSLDYWGQGTQVTVSS

SEQ ID NO: 79 - protein, 120 aa, Camelus bactrianus
QVQLQESGGGSVQAGGSLRLSCTASGFTFDDVDMGWYRQASGNECELVSTISSDGSTYYADSVKGRFTISQDNAKNTVYLQMNSLKPEDTAMYYCAAARYSDYEGMCGYWSQGTQVTVSS

SEQ ID NO: 80 - protein, 120 aa, Camelus bactrianus
QVQLQESGGGSVQAGGSLRLSCTASGFTFDDVDMGWYRQAPGNECELVSTISSDGSTYYADSVKGRFTISQDNAKNTVYLQMNSLKPEDTAMYYCAAARYSDYEGMCGYWSQGTQVTVSS

SEQ ID NO: 81 - protein, 120 aa, Camelus bactrianus
QVQLQESGGGSVQAGGSLRLSCTVSGFTFDDVDMGWYRQAPGNECELVSTISSDGSTYYADSVKGRFTISQDNAKNTVYLQMNSLKPEDTAMYYCAAARYSDYEGMCGYWSQGTQVTVSS

SEQ ID NO: 82 - protein, 120 aa, Camelus bactrianus
QVQLQESGGGLVQPGGSLRLSCAASGFTFDDVDMGWYRQAPGNECELVSTISSDGSTYYADSVKGRFTISQDNAKNTVYLQMNSLKPEDTAMYYCAAARYSDYEGMCGYWSQGTQVTVSS

SEQ ID NO: 83 - protein, 120 aa, Camelus bactrianus
QVQLQESGGGSVQAGESLRLSCRTSGFSFDDVDMGWYRQAPGNECELVSTISSDGSTYYADSVKGRFTISQDNAKNTVYLQMNSLKPEDTAMYYCAAARYSDYEGMCGYWSQGTQVTVSS

SEQ ID NO: 84 - protein, 120 aa, Camelus bactrianus
QVQLQESGGGSVQAGETLRLSCTVSGFTFDDADMGWYRQAPGNQCELVSTISSDGITYYADSVKGRFTVSQDNAKNTVYLQMNSLKPEDTAMYYCAAARYSDYEGMCGYWSQGTQVTVSS

SEQ ID NO: 85 - protein, 125 aa, Camelus bactrianus
QVQLQESGGGSVQAGGSLRLSCAASGFTYTGYCMGWFRQAPGKEREGVATVDSDGDTSYADSVKGRFTISKDNAKNTLYLQMNSLKPEDTAMYYCAADFSRWHLCSTSLATLGYWGQGTQVTVSS

SEQ ID NO: 86 - protein, 127 aa, Camelus bactrianus
QVQLQESGGGSVQAGGSLRLSCAASGYTYTGYCMGWFRQAPGKEREGVATIDSDGDTSYADSVKGRFTISKDNAKNTLYLQMNSLKPEDTAMYYCAADFRRWHLCSSSFREDGMDYWGKGTQVTVSS

SEQ ID NO: 87 - protein, 127 aa, Camelus bactrianus
QVQLQESGGGSVQAGETLRLSCAASGYTYTGYCMGWFRQATGKEREGVATIDSDGDTTYADSVKGRFTISKDNGKNTLYLQMNSLKPEDTAMYYCAADFRRWHLCSSSFQEYDMDYWGKGTQVTVSS

SEQ ID NO: 88 - protein, 118 aa, synthetic construct
  REGION 1..118: note = anti-HER2 VHH
EVQLVESGGGLVQAGGSLRLSCAASGITFSINTMGWYRQAPGKQRELVALISSIGDTYYADSVRGRFTISRDNAKNTVYLQMNSLKPEDTAVYYCKRFRTAAQGTDYWGQGTQVTVSS

SEQ ID NO: 89 - protein, 115 aa, synthetic construct
DVQLVESGGGLVQAGGSLRLSCAVSGSISSANSMGWYRQAPGKQRDVVAGLTTGGRSHYADSVKGRFTISRDNAKNTVYLQMNSLKAEDTAVYYCNLWPPVQGYWGQGTQVTVSS

SEQ ID NO: 90 - protein, 138 aa, synthetic construct
QVQLQESGGGLVQAGGSLRLSCTASGTFSGRNAMAWYRQAPGKRRELVAGIRSDDSTNYVDSVKGRFTISRDNAKNTVYLQMNSLRPEDTAVYYCNLWPPVIGAGFGSWGQGTQVTVSSAAAYPYDVPDYGSHHHHHH

SEQ ID NO: 91 - protein, 139 aa, synthetic construct
QVQLQESGGGTVQPGGSLRLSCAASGISVSIASMAWYRQAPGKQRELVAGIISGGSTNYADSVKGRFTISRDNAKKVVLLQMNSLKPEDTAVYYCNTYPPFVGPGATATWGQGTQVTVSSAAAYPYDVPDYGSHHHHHH

SEQ ID NO: 92 - protein, 135 aa, synthetic construct
QVQLQESGGGLVQSGGSLRLSCVASGSISSINAMGWYRQAPGNKREWVAGITSGGSTNYADAVLGRFSIFRDNPKNTVYLQMNSLKPEDTAVYYCNTWPPSTFGSWGQGTQVTVSSAAAYPYDVPDYGSHHHHHH

(19) United States
(12) Patent Application Publication
(10) Pub. No.: US 2025/0122304 A1
(43) Pub. Date: Apr. 17, 2025

(54) PRE-TARGETING

(71) Applicant: Precirix N.V., Brussels (BE)

(72) Inventors: Sophie Poty, Montpellier Cedex (FR); Jean-Pierre Pouget, Montpellier Cedex (FR); Tony Lahoutte, Brussels (BE); Matthias Bruno Lieven D'Huyvetter, Brussels (BE); Yana Dekempeneer, Brussels (BE); Francis Santens, Brussels (BE); Laurent Navarro, Brussels (BE)

(21) Appl. No.: 18/927,822

(22) Filed: Oct. 25, 2024

Related U.S. Application Data
(63) Continuation of application No. PCT/EP2023/061526, filed on May 2, 2023.

(30) Foreign Application Priority Data
May 2, 2022 (EP) 22305656.5

(51) Int. Cl. C07K 16/32; A61K 47/68; C07K 16/28; C07K 16/40

(52) U.S. Cl. CPC C07K 16/32; A61K 47/6803; A61K 47/6851; C07K 16/28; C07K 16/40; C07K 2317/33; C07K 2317/569; C07K 2317/92

(57) ABSTRACT

The present invention relates to the field of VHHs that may be used in pre-targeting combinations comprising the VHH and a labelled compound. Depending on the application, the labelled compound may comprise a radionuclide. The pre-targeting combinations are preferably for use in the treatment and/or diagnosis of a cancer.

### FIELD OF THE INVENTION

The present invention relates to the field of VHHs that may be used in pre-targeting combinations comprising the VHH and a labelled compound. Depending on the application, the labelled compound may comprise a radionuclide. The pre-targeting combinations are preferably for use in the treatment and/or diagnosis of a cancer.

### BACKGROUND

Cancers figure among the leading causes of morbidity and mortality worldwide. There is a continuous need for improved therapies combatting cancer while minimizing side effects. Radiolabeled heavy chain variable domain derived from a heavy chain antibody (VHH) have been proposed for such therapies. However, some of these therapies suffer from high kidney toxicities and low therapeutic indices (Parihar, Ashwin Singh, Sejal Chopra, and Vikas Prasad. “Nephrotoxicity after radionuclide therapies.” Translational Oncology 15.1 (2022): 101295.). Therefore, there is a need in the art for VHH radiotherapies for cancer with low kidney toxicities and high therapeutic indices.

### LEGEND TO THE DRAWINGS

FIG. 1A-D-Biodistribution of ¹⁷⁷Lu-VHH1 using a pre-targeting strategy up to 4 h post ¹⁷⁷Lu-DOTA-PEG₇-Tz injection using lag times of 30 min (FIG. 1A), 2 (FIG. 1B), 8 (FIG. 1C) and 24 (FIG. 1D) h. Data is expressed as % injected activity per gram of tissue (% IA/g), presented as mean±SD (n=5).

FIG. 2A-B—Biodistribution of ¹⁷⁷Lu-DOTA-VHH1 (FIG. 2A) and ¹⁷⁷Lu-DOTA-PEG₇-Tz (FIG. 2B) up to 4 h p.i. Data is expressed as % injected activity per gram of tissue (% IA/g), presented as mean±SD (n=5).

FIG. 3A-C-Statistical analysis of the biodistribution of ¹⁷⁷Lu-VHH1 using a pre-targeting strategy with different lag times, ¹⁷⁷Lu-DOTA-VHH1 and ¹⁷⁷Lu-DOTA-PEG₇-Tz 30 min p.i. (FIG. 3A), 1 h p.i. (FIG. 3B), and 4 h p.i. (FIG. 3C). Data is expressed as % injected activity per gram of tissue (% IA/g), presented as mean±SD (n=5).

FIG. 4A-C-Statistical analysis of the biodistribution of ¹⁷⁷Lu-VHH1 using a pre-targeting strategy with different molecular masses and ¹⁷⁷Lu-DOTA-PEG₇-Tz 30 min p.i. (FIG. 4A), 1 h p.i. (FIG. 4B), and 4 h p.i. (FIG. 4C). Data is expressed as % injected activity per gram of tissue (% IA/g), presented as mean±SD (n=5).

FIG. 5A-C-Statistical analysis of the biodistribution in kidneys (FIG. 5A) and tumor (FIG. 5B) of ¹⁷⁷Lu-VHH1 using a pre-targeting strategy with a 4 h and 8 h lag time and ¹⁷⁷Lu-DOTA-VHH1 up to 96 h p.i. Data is expressed as % injected activity per gram of tissue (% IA/g), presented as mean±SD (n=5). (FIG. 5C) The tumor-to-kidney ratio of of ¹⁷⁷Lu-VHH1 using a pre-targeting strategy with a 4 h and 8 h lag time and ¹⁷⁷Lu-DOTA-VHH1 up to 96 h p.i.

FIG. 6A-C—The biodistribution profiles describing the uptake of ¹⁷⁷Lu-labeled-Tz using a low (FIG. 6A) or a high (FIG. 6B) specific activity. Data in A and B is expressed as % injected activity per gram of tissue (% IA/g), presented as mean±SD (n=5). FIG. 6C reflects the resulting AUCs for both strategies.

FIG. 7A-C—The therapeutic efficacy of pre-targeted ²²⁵Ac-VHH1. (FIG. 7A) The Kaplan-Meier curve describing the survival of mice treated with pre-targeted ²²⁵Ac-VHH1 versus direct labeled ²²⁵Ac-DOTA-VHH1. The red arrows indicate the 6 consecutive treatments. (FIG. 7B) Tumor growth dynamics after treatment. High dose pre-targeted ²²⁵Ac-VHH1 and direct labeled ²²⁵Ac-DOTA-VHH1 are effective in mice with established subcutaneous human glioblastoma tumors (U87 MG) as revealed by (A) the resulting extended survival of treated mice and their (B) capacity to inhibit tumor development. (FIG. 7C) Weight loss after treatment. MS=mean survival, (n=10 per treatment group).

FIG. 8A-B—Long-term toxicology follow-up of healthy female C57BI/6 mice treated with pre-targeted ²²⁵Ac-VHH1. (FIG. 8A) The Kaplan-Meier curve describing the survival of mice treated with pre-targeted ²²⁵Ac-VHH1 or direct labeled ²²⁵Ac-DOTA-VHH1. The red arrows indicate the 6 consecutive treatments. (FIG. 8B) Weight progression after treatment. N=5 per treatment group for 6 months follow-up.

### DETAILED DESCRIPTION OF THE INVENTION

### VHH or Fragment Thereof

In a first aspect of the invention, there is provided a heavy chain variable domain derived from a heavy chain antibody (VHH), or a fragment thereof, comprising a click group for use as a medicament, preferably for use in the treatment and/or diagnosis of a cancer. Preferably, said click group has not underwent a click reaction upon administration of said medicament to a patient.

In a second aspect of the invention, there is provided a heavy chain variable domain derived from a heavy chain antibody (VHH), or a fragment thereof, comprising a click group, wherein said VHH, or fragment thereof, is able to specifically bind an antigen with a dissociation constant (K_{off}) equal to or smaller than 10⁻¹ s⁻¹, preferably equal to or smaller than 10⁻² s⁻¹, more preferably equal to or smaller than 10⁻³ s⁻¹, most preferably equal to or smaller than 10⁻⁴ s⁻¹; preferably for use as a medicament, more preferably for use in the treatment and/or diagnosis of a cancer associated with said antigen. Preferably, said click group has not underwent a click reaction upon administration of said medicament to a patient.

Wherever a heavy chain variable domain derived from a heavy chain antibody (VHH) a heavy chain antibody (VHH), or a fragment thereof, is mentioned in this application, reference is made to a heavy chain antibody (VHH), or a fragment thereof, comprising a click group as defined in this first or second aspect, unless explicitly mentioned otherwise. Such a VHH, or a fragment thereof, may also be called a VHH, or a fragment thereof, according to the invention. Herein, it is understood that a VHH, or a fragment thereof, according to the invention is preferably for use in the treatment and/or diagnosis of a cancer.

In embodiments, the VHH's as disclosed herein are able to specifically bind an antigen with a dissociation constant (K_{off}) in the range from 10⁻¹ s⁻¹ to 10⁻⁵ s⁻¹, preferably in the range from 10⁻² s⁻¹ to 10⁻⁵ s⁻¹, more preferably in the range from 10⁻³ s⁻¹ to 10⁻⁵ s⁻¹, most preferably in the range from 10⁻⁴ s⁻¹ to 10⁻⁵ s⁻¹. Example 7 shows the suitability of limited K_{off} rates for the pretargeting approach.

The heavy chain variable domains derived from heavy chain antibodies (VHH's), or fragments thereof, as disclosed herein consist of a single polypeptide chain. More particularly, the VHH's are derived from an innate or adaptive immune system, preferably from a protein of an innate or adaptive immune system. Still more particularly, the VHH's disclosed herein may comprise 4 framework regions (FR) and 3 complementary determining regions (CDR), or any suitable fragment thereof (which will then usually contain at least some of the amino acid residues that form at least one of the CDR). In particular, the VHH's disclosed herein are easy to produce at high yield, preferably in a microbial recombinant expression system, and convenient to isolate and/or purify subsequently.

The VHH's as disclosed herein may comprise CDR (complementarity determining regions) sequences of antibodies (or may be based on and/or derived from such CDR sequences, as further described herein), they will also generally be referred to herein as ‘CDR sequences’ (i.e. as CDR1 sequences, CDR2 sequences and CDR3 sequences, respectively). In an embodiment, the VHH's as disclosed herein comprise at least one amino acid sequence that is chosen from the group consisting of the CDR1 sequences, CDR2 sequences and CDR3 sequences that are described herein.

In an embodiment, the VHH's as disclosed herein have the (general) structure: FR1-CDR1-FR2-CDR2-FR3-CDR3-FR4

Within the context of the invention the IMGT nomenclature is used to define the FR (framework regions) FR1, FR2, FR3 and FR4 and corresponding CDR regions CDR1, CDR2, and CDR3. The definition of the IMGT nomenclature used is provided later herein in the general part dedicated to the definition of the invention.

A fragment of a VHH preferably exhibits at least an activity of the VHH to some extent. “Some extent” may mean at least 40%, 50%, 60%, 70%, 80%, 90%, 95%, 100% or more. A preferred activity of a fragment of a VHH is the specific binding to an antigen, preferably with a dissociation constant (K_{off}) equal to or smaller than 10⁻¹ s⁻¹, preferably equal to or smaller than 10⁻² s⁻¹, more preferably equal to or smaller than 10⁻³ s⁻¹, most preferably equal to or smaller than 10⁻⁴ s⁻¹. Preferred antigens are described below. A more preferred activity of a fragment of a VHH is the specific binding to an antigen with a dissociation constant (K_{off}) in the range from 10⁻¹ s⁻¹ to 10⁻⁵ s⁻¹, preferably in the range from 10⁻² s⁻¹ to 10⁻⁵ s⁻¹, more preferably in the range from 10⁻³ s⁻¹ to 10⁻⁵ s⁻¹, most preferably in the range from 10⁻⁴ s⁻¹ to 10⁻⁵ s⁻¹.

A fragment of a VHH preferably has a length from 20 to 50, 55, 60, 65, 70, 75, 80, 85, 90, 95, 100, 105, 110, 115, 120, 125, 130, 135, 140, 145, or 150 amino acids, or from 50 to 80, 85, 90, 95, 100, 105, 110, 115, 120, 125, 130, 135, 140, 145 or 150 amino acids.

In embodiments, a VHH, or a fragment thereof, according to the invention comprises from 1 to 20, 19, 18, 17, 16, 15, 14, 13, 12, 11, 10, 9, 8, 7, 6, 5, 4, 3, or 2 click groups. In embodiments, a VHH, or a fragment thereof, according to the invention comprises more than 1 click group. In embodiments, a VHH, or a fragment thereof, according to the invention comprises from 2 to 20, 19, 18, 17, 16, 15, 14, 13, 12, 11, 10, 9, 8, 7, 6, 5, 4, or 3 click groups. In embodiments, a VHH, or a fragment thereof, according to the invention comprises from 3 to 20, 19, 18, 17, 16, 15, 14, 13, 12, 11, 10, 9, 8, 7, 6, 5, or 4 click groups. In embodiments, a VHH, or a fragment thereof, according to the invention comprises 1, 2, 3, 4, 5, 6, 7, 8, 9, 10, 11, 12, 13, 14, 15, 16, 17, 18, 19, 20 click groups.

In embodiments, all click groups comprised in a VHH, or a fragment thereof, according to the invention are of the same type. According to a preferred definition, two click groups are of the same type if they are conformers, tautomers, isotopologues or isotopomers of each other.

The first aspect of this invention relates to “a heavy chain antibody (VHH), or a fragment thereof, comprising a click group”. It should be understood that “comprising a click group” may refer to either the click group being conjugated to the VHH, or to the click group being incorporated into the VHH in other ways, e.g. by virtue of a modified amino acid.

In embodiments, the click group comprised in a VHH, or a fragment thereof according to the invention, is conjugated to the VHH.

In embodiments, the click groups comprised in a VHH, or a fragment thereof, according to the invention are conjugated to said VHH at a lysine residue, a cysteine residue, a glycan, a N-terminus or a C-terminus comprised in said VHH, preferably at a lysine residue, a N-terminus or a C-terminus comprised in said VHH.

In embodiments, the click groups are conjugated to a VHH, or a fragment thereof, according to the invention by reacting a corresponding VHH, which has not been conjugated to said click groups, with a compound comprising a first group, which is able to conjugate with a lysine residue, a cysteine residue, a glycan, a N-terminus or a C-terminus, preferably a lysine residue, a N-terminus or a C-terminus, comprised in said VHH, and one of said click groups. Preferably, said reaction takes places in an aqueous buffer, more preferably in a basic aqueous buffer. Preferably, said first group is a N-hydroxysuccinimide (NHS), more preferably said first group is a N-hydroxysuccinimide (NHS) and (each of) said click group(s) is TCO.

### Combination

In an aspect of the invention, there is provided a combination for use in the treatment and/or diagnosis of a cancer, comprising:
a heavy chain variable domain derived from a heavy chain antibody (VHH), or a fragment thereof, according to the invention; and
a labelled compound, comprising a label and a click group;

wherein said VHH, or fragment thereof, is able to specifically bind an antigen associated with said cancer with a dissociation constant (k_{off}) equal to or smaller than 10⁻¹ s⁻¹, preferably equal to or smaller than 10⁻² s⁻¹, more preferably equal to or smaller than 10⁻³ s⁻¹, most preferably equal to or smaller than 10⁻⁴ s⁻¹;

wherein said use comprises a first administration of said VHH, or fragment thereof, and a subsequent administration of said labelled compound to a subject in need thereof;

wherein said click group comprised in said VHH, or fragment thereof, is able to undergo a click reaction in said subject with said click group comprised in said labelled compound.

Such a combination may be called a combination according to the invention, wherein it is understood that said combination is for use in the treatment and/or diagnosis of a cancer.

In embodiments, the VHH's comprised in a combination according to the invention are able to specifically bind an antigen with a dissociation constant (K_{off}) in the range from 10⁻¹ s⁻¹ to 10⁻⁵ s⁻¹, preferably in the range from 10⁻² s⁻¹ to 10⁻⁵ s⁻¹, more preferably in the range from 10⁻³ s⁻¹ to 10⁻⁵ s⁻¹, most preferably in the range from 10⁻⁴ s⁻¹ to 10⁻⁵ s⁻¹.

Without being bound to this theory, the use of a combination according to the invention results in the selective or specific delivery of the label to a cell, a tissue, or an organ (over) expressing the antigen.

It is understood that any VHH, or fragment thereof, according to the invention disclosed in this application, or any labelled compound disclosed in this application, may be comprised in a combination according to the invention. Hence, all preferred embodiments below may also be envisaged as combinations according to the invention.

In embodiments, a combination according to the invention is a kit or a kit-of-parts, wherein the VHH or fragment thereof and the labelled compound comprised therein are present as separate parts or components. Separate parts or components in this context preferably means that the VHH (or fragment thereof) and the labelled fragment are not present in a single chemical composition, as understood by the skilled person. This implies that the components are designed for separate administration (i.e. first and subsequent administration as described above). In other words, a combination should not be confused with a composition, as the latter implies a single chemical composition.

Conventional radioimmunotherapy and diagnosis, for which detailed protocols are readily available to the expert (Cancer Radiotherapy: Methods and Protocols (Methods in Molecular Medicine), Huddart RA Ed., Human Press 2002), involves the ex vivo labeling of a targeting moiety (such as a VHH or a fragment thereof), followed by a single administration of the resulting label-targeting moiety conjugate. Herein, the label may be a diagnostic or a therapeutic compound, as described below. In other words, the specific binding of a clinical target (such as an antigen associated with a cancer) is concurrent with the delivery of a therapeutically or diagnostically active substance (the label).

On the other hand, the use of a combination according to the invention in the treatment and/or diagnosis of a cancer involves two, subsequent (and thus separate) administration steps, wherein the first administration is of said VHH, or fragment thereof (resulting in specific binding of the antigen), and the second administration is of said labelled compound comprising the label (resulting in a click reaction between the VHH or fragment and the labelled compound). In other words, the specific binding and the delivery of the therapeutically or diagnostically active substance are decoupled. As such, the use of a combination according to the invention may be called “pre-targeting” and the combination may be a called a “pre-targeting combination” in the context of this invention.

Preferably, the click reaction between said click group comprised in said VHH, or fragment thereof, and said click group comprised in said labelled compound preferably does not lead to undesired side reaction with biomolecules, i.e. is a specific reaction. As such, without being bound to this theory, the specific binding of the VHH or fragment, which is the result of the first administration, translates to a specific delivery of the labelled compound during the subsequent administration.

Without being bound to this theory, an advantage of the pre-targeting use of a combination according to the invention compared to the use of a radiolabelled VHH, or a fragment thereof, using conventional radioimmunotherapy and diagnosis characterized by a single administration in the treatment or diagnosis of a cancer is one or more of the following:
reduced retention of the label in the kidneys, preferably by at least 5%, 10%, 20%, 30%, 40%, 50%, 60%, 70%, 80%, 90%, 100%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%;
increased therapeutic index of the label, preferably by at least 5%, 10%, 20%, 30%, 40%, 50%, 60%, 70%, 80%, 90%, 100%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%;

Preferably, said advantages are obtained while maintaining tumor targeting potential, as explained in Example 3.

In embodiments, the time between the first administration and the subsequent administration is around 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 days, preferably wherein around X days means from 0.8 times X days up to 1.2 times X days.

In embodiments, the time between the first administration and the subsequent administration is around 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 hours, preferably wherein around X hours means from 0.8 times X hours up to 1.2 times X hours.

In embodiments, the time between the first administration and the subsequent administration is around 10, 20, 30, 40, 50, 60, 70, 80, 90, 100, 110, 120, 130, 140, 150, 160, 170, 180, 190, 200, 210, 220, 230, or 240 minutes, preferably wherein around X minutes means from 0.8 times X minutes up to 1.2 times X minutes.

In embodiments, the time between the first administration and the subsequent administration are within 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 days, preferably wherein the time between the first and the subsequent administration is at least 1 day, more preferably at least 2 days.

In embodiments, the time between the first administration and the subsequent administration are within 72, 71, 70, 69, 68, 67, 66, 65, 64, 63, 62, 61, 60, 59, 58, 57, 56, 55, 54, 53, 52, 51, 50, 49, 48, 47, 46, 45, 44, 43, 42, 41, 40, 39, 38, 37, 36, 35, 34, 33, 32, 31, 30, 29, 28, 27, 26, 25, 24, 23, 22, 21, 20, 19, 18, 17, 16, 15, 14, 13, 12, 11, 10, 9, 8, 7, 6, 5, 4, 3, 2 or 1 hours, preferably wherein the time between the first and the subsequent administration is at least 1 hour, more preferably at least 2 hours.

In embodiments, the time between the first administration and the subsequent administration are within 240, 230, 220, 210, 200, 190, 180, 170, 160, 150, 140, 130, 120, 110, 100, 90, 80, 70, 60, 50, 40, 30, 20, or 10 minutes, preferably wherein the time between the first administration and the subsequent administration is at least 10 minutes.

In embodiments, the time between the first administration and the subsequent administration is at least 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 days, preferably wherein the time between the first administration and the subsequent administration is less than 30 days.

In embodiments, the time between the first administration and the subsequent administration is at least 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, or 72 hours, preferably wherein the time between the first administration and the subsequent administration is less than 72 hours.

In embodiments, the time between the first administration and the subsequent administration is at least 10, 20, 30, 40, 50, 60, 70, 80, 90, 100, 110, 120, 130, 140, 150, 160, 170, 180, 190, 200, 210, 220, 230, or 240 minutes, preferably wherein the time between the first administration and the subsequent administration is less than 240 minutes.

In the context of this invention, the time between the first and the subsequent administration may be called the lag time.

A lag time optimization for the pretargeting using TCO-VHH1 (first administration) and ¹⁷⁷Lu-DOTA-PEG₇-Tz (second administration) has been performed in Example 1. Similar protocols can be performed for other applications.

The use of pretargeting combinations comprising monoclonal antibodies, comprising a click group, and a labelled compound is already described in the literature. In the case of monoclonal antibodies, however, the use of pretargeting combinations addresses the issue of a high toxicity due to a long blood circulation. Since monoclonal antibodies typically have a long circulation time, their radiolabelled counterparts remain in circulation for a long time after a direct administration, causing toxicity issues. The pretargeting approach is able to overcome this problem by first administering unlabelled monoclonal antibodies, then administering a clearing agent to effectively take the unlabelled monoclonal antibodies which have not bound specifically to their targets out of circulation, and subsequently administering the labelled compound which is able to bind specifically to any monoclonal antibodies which have been specifically bound to their targets (i.e. tumors). Since the labelled compound generally does not remain in circulation for a long period of time, this pretargeting approach overcomes the toxicity issues.

In contrast, the use of pretargeting combinations according to the invention comprise a heavy chain antibody (VHH), or a fragment thereof. These targeting compounds are not characterized by a long circulation time.

In fact, they are cleared rapidly from blood stream in comparison to monoclonal antibodies. As a result, they are less hampered by the general toxicity issues associated with radiolabeled monoclonal antibodies. As a result, the skilled person would not envision using the pretargeting approach for VHHs, or fragments thereof, as it is unlikely to add any value or solve a particular problem associated with their use. In the present case, the pretargeting approach addresses the kidney toxicity issues associated with the rapid clearance of VHHs, or fragments thereof, an effect which is not evident from the prior art. In other words, it would not be obvious for a skilled person to use the pretargeting approach for VHHs, or fragments thereof, based on the publicly available knowledge about pretargeting for monoclonal antibodies.

Furthermore, the use of pretargeting combinations according to the invention typically does not involve the use of a blood clearing agent, as this agent only serves a purpose in the context of monoclonal antibodies with long circulation times. In fact, the absence of the administration of an additional agent may be a benefit of the use of pretargeting combinations according to the invention. Clearly, the possibility to leave out the blood clearing agent would not be obvious for the skilled person based on the publicly available knowledge about pretargeting for monoclonal antibodies.

In embodiments, the use of a combination according to the invention does not comprise the administration of a blood clearing agent to the subject between the first administration and the subsequent administration. A blood clearing agent as used herein is an agent capable of and/or with the purpose of removing or clearing unbound VHH from blood circulation of the subject, which is typically administered between the first and subsequent administration in the context of pretargeting. Examples of blood clearing agents include, without being limiting, avidin, galactose, biotinylated N-acetyl-galactosamine and binders, such as antibodies and fragments thereof, targeted against the VHH. Without being bound to this theory, the first administration may result in some fraction of the VHH, or fragment thereof, not specifically bound to the antigen, in the blood circulation of the subject. Some fraction is for example from 0% up to 0.1%, 0.5%, 1%, 2%, 3%, 4%, 5%, 6%, 7%, 8%, 9%, 10%, 11%, 12%, 13%, 14%, 15%, 16%, 17%, 18%, 19%, or 20% of the VHH, or fragment thereof, which has been administered. A preferred advantage of the use of a combination according to the invention is that no blood clearing agent needs to be administered between the first administration and the subsequent administration to achieve the therapeutic or diagnostic effect as described below.

In embodiments, the use of a combination according to the invention does not comprise the administration of biotinylated N-acetyl-galactosamine.

It is understood that any preferred VHH, or fragment thereof, according to the invention, as described below, should also be understood as a preferred combination according to the invention comprising the preferred VHH, or fragment thereof. Wherever reference is made to the administration of a combination according to the invention in this application, both the first administration and the subsequent administration are referred to.

### Click Groups and Pairs

Click chemistry is a chemical approach introduced by Sharpless in 2001 and well-known to the skilled person. According to a preferred definition, a click (chemistry) reaction comprises the joining of small-molecule modular groups (i.e. (part of) the reactants), which may be attached to larger moieties such as biomolecules, with a high rate, yield and specificity (e.g. stereospecificity). More preferably, undesired side products are only formed in a limited amount and may be removed from the resulting reacting mixture with limited efforts, preferably without the use of chromatography. More preferably, a click reaction is relatively insensitive to solvent parameters and the presence of oxygen and/or water. Most preferably, a click reaction is biocompatible, i.e. does not cause undesired side reaction with biomolecules and/or does not result in the formation of compounds that are toxic and/or not biocompatible.

Examples of click reactions can be found in Kolb, Finn and Sharpless Angewandte Chemie International Edition (2001) 40:2004-2021; Evans, Australian Journal of Chemistry (2007) 60:384-395). Click reactions include, but are not limited to, formation of esters, thioesters, amides (e.g., such as peptide coupling) from activated acids or acyl halides; nucleophilic displacement reactions (e.g., such as nucleophilic displacement of a halide or ring opening of strained ring systems); azide-alkyne Huisgen cycloaddition (e.g., 1,3-dipolar cycloaddition between an azide and an alkyne to form a 1,2,3-triazole linker); thiol-yne addition; imine formation; Diels-Alder reactions between tetrazines and trans-cyclooctene (TCO); and Michael additions (e.g., maleimide addition).

Click chemistry reactions between alkynes and azides typically require the addition of a copper catalyst to promote the 1,3-cycloaddition reaction, and are known as copper-catalyzed azide-alkyne cycloaddition (CuAAC) reactions. However, click chemistry reactions between cyclooctyne or cyclooctyne derivatives and azides typically do not require the addition of a copper catalyst, and instead proceed via strain-promoted azide-alkyne cycloaddition (SPAAC).

As used herein, a “click group” refers to a moiety that can partake as a modular group in a click chemistry reaction, i.e. as (part of) a reactant. A click group can be a moiety that is rarely found in naturally-occurring biomolecules and is chemically inert towards biomolecules, but, e.g., when reacted with an azide-reactive or alkyne-reactive group, the reaction can take place efficiently under biologically relevant conditions, for example in cell culture conditions, such as in the absence of excess heat or harsh reactants. Preferably, a click group is biocompatible.

In this section, preferred click groups are described. It is understood that these preferences apply to a click group comprised in a VHH, or a fragment thereof, according to the invention; and to a click group comprised in a labelled compound comprised in a combination according to the invention.

In embodiments, a click group is an alkene, a diene, an alkyne, an azide, a nitrone, a tetrazine, or a sydnone.

In embodiments, there is provided a VHH, or a fragment thereof, according to the invention, wherein the click group is an alkene, an alkyne, an azide, a nitrone, or a tetrazine, preferably wherein said alkene is a trans-cyclooctene.

In embodiments, there is provided a combination according to the invention, wherein the click group comprised in the labelled compound is an alkene, an alkyne, an azide, a nitrone, or a tetrazine, preferably wherein said alkene is a trans-cyclooctene, more preferably wherein said click group comprised in said labelled compound is a tetrazine.

As used herein, the term “alkene” refers to an unsaturated hydrocarbon moiety or molecule that includes a carbon-carbon double bond. A preferred alkene is trans-cyclooctene (TCO). Preferred alkenes have a number of carbon atoms from 2 to 100, to 95, to 90, to 85, to 80, to 75, to 70, to 65, to 60, to 55, to 50, to 45, to 40, to 35, to 30, to 25, to 20, to 19, to 18, to 17, to 16, to 15, to 14, to 13, to 12, to 11, to 10, to 9, to 8, to 7, to 6, or to 5.

As used herein, the term “diene” refers to a moiety or molecule having two double bonds where these double bonds are conjugated in the 1,3-position. The double bonds of the diene can be either cis or trans. Preferred dienes have a number of nitrogen atoms of 1, 2, 3, 4, 5 or 6. Preferred alkenes have a number of carbon atoms from 1 to 100, to 95, to 90, to 85, to 80, to 75, to 70, to 65, to 60, to 55, to 50, to 45, to 40, to 35, to 30, to 25, to 20, to 19, to 18, to 17, to 16, to 15, to 14, to 13, to 12, to 11, to 10, to 9, to 8, to 7, to 6, or to 5. Preferred dienes comprise 1, 2, 3, 4 or 5 covalent nitrogen-nitrogen bonds. Preferred dienes are tetrazines.

As used herein, the term “alkyne” refers to a moiety or molecule comprising a carbon-carbon triple bond. Alkyne moieties include terminal alkynes and cyclic alkynes, preferably terminal alkynes and cyclic alkynes that are reactive with azide groups. A terminal alkyne has at least one hydrogen atom bonded to a triply bonded carbon atom. A cyclic alkyne is a cycloalkyl ring comprising one or more triple bonds. Preferred alkenes are cyclic alkenes. Preferred cyclic alkynes are cyclooctyne and cyclooctyne derivatives, such as bicyclononyne (BCN), biarylazacyclooctynone (BARAC), and dibenzoazacyclooctyne (DIBAC).

As used herein, the term “azide” refers to a moiety or molecule comprising a —N₃ functional group. Preferred azides have a number of carbon atoms from 1 to 100, to 95, to 90, to 85, to 80, to 75, to 70, to 65, to 60, to 55, to 50, to 45, to 40, to 35, to 30, to 25, to 20, to 19, to 18, to 17, to 16, to 15, to 14, to 13, to 12, to 11, to 10, to 9, to 8, to 7, to 6, or to 5. A preferred azide is an NHS-azide.

As used herein, the term “nitrone” refers to a moiety or molecule comprising a >C═N⁺(—O⁻)— functional group, preferably comprising —C═N⁺(—O⁻)—H. Preferred nitrones have a number of carbon atoms from 1 to 100, to 95, to 90, to 85, to 80, to 75, to 70, to 65, to 60, to 55, to 50, to 45, to 40, to 35, to 30, to 25, to 20, to 19, to 18, to 17, to 16, to 15, to 14, to 13, to 12, to 11, to 10, to 9, to 8, to 7, to 6, or to 5.

As used herein, the term “tetrazine” refers to a six-membered aromatic ring comprising exactly four nitrogen atoms. Preferred tetrazines have a number of carbon atoms from 2 to 100, to 95, to 90, to 85, to 80, to 75, to 70, to 65, to 60, to 55, to 50, to 45, to 40, to 35, to 30, to 25, to 20, to 19, to 18, to 17, to 16, to 15, to 14, to 13, to 12, to 11, to 10, to 9, to 8, to 7, to 6, or to 5. Preferred tetrazines are 1,2,4,5-tetrazines.

Click reactions require at least two click groups that are able to react with each other. In this context, click groups able to react with each other may be called a click (chemistry) pair. The click groups constituting a click pair may be called click pair partners. “To be able to undergo a click reaction”, and similar expressions, are synonymous with “to form a click pair”, and similar expressions, in the context of this application.

An open click group is a click group which has not underwent a click reaction. In other words, an open click group is a click group which is still able to undergo a click reaction with another click group.

A closed click group is a click group which has underwent a click reaction. In other words, a closed click group is a moiety which is bound, typically covalently bound, to its click pair partner.

In this section, preferred click pairs are described. It is understood that the preference for a click pair consisting of click group A and click group B implicates a preferred combination according to the invention, wherein the VHH, or a fragment thereof, according to the invention comprises one or more click groups A and the labelled compound comprises click group B; and a preferred combination according to the invention, wherein the VHH, or a fragment thereof, according to the invention comprises one or more click groups B and the labelled compound comprises click group A.

In some embodiments, a click pair consists of an azide and an alkyne, preferably a cyclic alkyne such as BCN, BARAC or DIBAC.

In some embodiments, a click pair consists of a tetrazine and an alkene or alkyne, preferably a cyclic alkene or alkyne. In this context, a cyclic alkene is preferably a trans-cyclooctene. In this context, a cyclic alkyne is preferably BCN, BARAC or DIBAC.

### Labels

A combination according to the invention comprises a labelled compound, comprising a label and a click group. Below, preferred labels are provided. It is understood that these preferences imply preferred corresponding combinations according to the invention.

In embodiments, a label is a therapeutic or diagnostic group, preferably useful in the treatment or diagnosis of a cancer as discussed herein.

In embodiments, a label is a small molecule. Preferably, a small molecule is defined as an organic molecule or corresponding moiety having a molecular weight of at most 2000 Da, preferably at most 1500 Da, more preferably at most 1000 Da such as at most 900 Da.

### Radionuclides

In embodiments, a label is a radionuclide. In this context, a corresponding labelled compound comprising a label which is a radionuclide may be called a radiolabelled compound.

In embodiments, a label is a radionuclide suitable for therapeutic applications, preferably a radionuclide chosen from the group consisting of α-emitting radioisotopes and β-emitting radioisotopes, including but not limited to a radioisotope chosen from the group consisting of actinium-225, astatine-211, bismuth-212, bismuth-213, caesium-137, chromium-51, cobalt-60, copper-67, dysprosium-165, erbium-169, fermium-255, gold-198, holium-166, iodine-125, iodine-131, iridium-192, iron-59, lead-212, lutetium-177, molybdenum-99, palladium-103, phosphorus-32, potassium-42, rhenium-186, rhenium-188, samarium-153, radium-223, radium-224, ruthenium-106, scandium-47, sodium-24, strontium-89, terbium-149, terbium-161, thorium-227, xenon-133, ytterbium-169, ytterbium-177 and yttrium-90. In more preferred embodiments, a label is iodine-131. In preferred embodiments, a label is chosen from the group consisting of actinium-225, bismuth-213, iodine-125, iodine-131, lutetium-177, yttrium-90, copper-67, rhenium-186, rhenium-188, terbium-149, terbium-161, astatine-211 or fluorine-18. In more preferred embodiments, a label is astatine-211 or fluorine-18. In more preferred embodiments, a label is actinium-225 or bismuth-213, most preferably actinium-225. In more preferred embodiments, a label is lutetium-177 or terbium-161, most preferably lutetium-177.

In embodiments, a label is a radionuclide suitable for diagnostic applications, preferably a radionuclide chosen from the group consisting of positron-emitting radioisotopes (PET) or γ-emitting radioisotopes (SPECT), including but not limited to a radioisotope chosen from the group consisting of iodine-131, yttrium-90, iodine-125, lutetium-177, lead-203, rhenium-186, rhenium-188, scandium-43, scandium-44, technetium-99m, terbium-161, terbium-149, indium-111, xenon-133, thallium-201, fluorine-18, gallium-68, gallium-67, copper-67, iodine-123, iodine-124, zirconium-89 and copper-64.

In more preferred embodiments, a label is iodine-131. In more preferred embodiments, a label is actinium-225 or bismuth-213, most preferably actinium-225. In more preferred embodiments, a label is lutetium-177 or terbium-161, most preferably lutetium-177.

In embodiments, a label is a radionuclide that is able to form a non-covalent, coordinative bond with a chelator, as described below, selected from the group consisting of actinium-225, bismuth-212, bismuth-213, caesium-137, chromium-51, cobalt-60, copper-67, erbium-169, fermium-255, gold-198, iron-59, lead-212, lutetium-177, holium-166, potassium-42, rhenium-186, rhenium-188, samarium-153, radium-223, radium-224, scandium-47, sodium-24, terbium-149, and terbium-161.

In embodiments, a label is a radionuclide that is able to form a non-covalent, coordinative bond with DOTA, as described below, selected from the group consisting of actinium-225, bismuth-212, bismuth-213, copper-67, erbium-169, iron-59, lead-212, lutetium-177, samarium-153, radium-223, radium-224, scandium-47, terbium-149, terbium-161, thorium-227, ytterbium-169, ytterbium-177, and yttrium-90.

In embodiments, a label is a radionuclide that can bind covalent to a linker, as described below, selected from the group consisting of astatine-211, dysprosium-165, iodine-125, iodine-131, iridium-192, molybdenum-99, palladium-103, phosphorus-32, ruthenium-106, strontium-89, and xenon-133.

### Other Labels

In embodiments, a label is a non-radioactive label. In an embodiment, such non-radioactive label is a fluorescent label. Such a label, and corresponding combinations according to the invention, may be used for diagnostic applications as defined herein. Alternative applications include image-guided surgery or photodynamic therapy. Examples of suitable fluorescent labels for diagnostic applications include Alexa fluor variants, Cy3, Cy5, FITC (fluorescein), Coumarin, Texas red, Oregon Green, Pacific Blue, Pacific Green, Pacific Orange, PE-Cyanine7, PerCP-Cyanine5.5, TRITC (tetramethylrhodamine). Examples of suitable fluorescent labels for image-guided surgery include IRDye800CW, IRDye680-RD, ZW800-1, FNIR (see for example Pieterjan Debie et al, Front Pharmacology, 2019; 10:510, doi: 10.3389/fphar.2019.00510, PMCID: PMC6527780, PMID: 31139085). Example of a suitable fluorescent label for photodynamic therapy includes IRDye700DX. Most labels may be obtained from ThermoFisher or from Licor.

In embodiments, a label is a molecule to be delivered to a cell, a tissue, an organ expressing human and/or murine FAP. Any moiety, molecule or medicament known to act on a cell, tissue, organ expressing FAP is potentially encompassed by these embodiments. The molecule may be a peptide, a small molecule or a nucleic acid. A peptide may be a cytokine. A small molecule may be a chemotherapeutic. An entity may be a cell such as a CAR-T cell, a CAR-NK cell, a BITE or a LITE.

In embodiments, a label is an AcTakine (Activity-on-Target cytokine) or an AcTaferon (IFNα-based AcTakine), preferably an AcTakine or an AcTaferon as described in WO2017077382A1, WO2017134301A1, WO2017194783A1, WO2017194782A2, WO2018077893A1, WO2018141964A1, WO2018144999A1, WO2019032661A1, WO2019032663A1, WO2019032662A1, WO2019148089A1, WO2019191519A1 or WO2020033646A1. In these embodiments, the label is preferably a medicament for cancer.

In embodiments, a label is a pyrrolobenzodiazepine; preferably a pyrrolobenzodiazepine dimer such as described in WO2014057074A1, WO2015052322A1, WO2014140174A1, WO2015052321A1, WO2017186894A1, WO2017137555A1, WO2017137553A1, WO2016038383A1 or WO2018192944A1; more preferably herein said pyrrolobenzodiazepine dimer is selected from the group consisting of:
(11S,11aS)-4-((2R,5R)-37-(2,5-dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl 11-hydroxy-8-((5-(((11S,11aS)-11-hydroxy-10-(((4-((10R,13R)-10-isopropyl-13-methyl-8,11-dioxo-2,5-dioxa-9,12-diazatetradecanamido)benzyl)oxy)carbonyl)-7-methoxy-2-methyl-5-oxo-5,10,11,11a-tetrahydro-1H-pyrrolo[2,1-c][1,4]benzodiazepin-8-yl)oxy)pentyl)oxy)-7-methoxy-2-methyl-5-oxo-11,11a-dihydro-1H-pyrrolo[2,1-c][1,4]benzodiazepine-10(5H)-carboxylate,
(S)-3-(2,5-dioxo-2,5-dihydro-1H-pyrrol-1-yl)-N-(2-(2-(2-(2-(4-(((8-methoxy-2-(6-methoxynaphthalen-2-yl)-5-oxo-5,11a-dihydro-1H-benzo[e]pyrrolo[1,2-a][1,4]diazepin-7-yl)oxy)methyl)-1H-1,2,3-triazol-1-yl)ethoxy)ethoxy)ethoxy)ethyl)propanamide,
(S)-3-(2,5-dioxo-2,5-dihydro-1H-pyrrol-1-yl)-N-(2-(2-(2-(2-(4-(((2-methylene-5-oxo-2,3,5,11a-tetrahydro-1H-benzo[e]pyrrolo[1,2-a][1,4]diazepin-7-yl)oxy)methyl)-1H-1,2,3-triazol-1-yl)ethoxy)ethoxy)ethoxy)ethyl)propanamide,
1-(3-(2,5-dioxo-2,5-dihydro-1H-pyrrol-1-yl) propanamido)-N-(3-(((S)-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-5,11a-dihydro-1H-benzo[e]pyrrolo[1,2-a][1,4]diazepin-7-yl)oxy) propyl)-3,6,9,12,15,18,21,24-octaoxaheptacosan-27-amide,
3-(2,5-dioxo-2,5-dihydro-1H-pyrrol-1-yl)-N-(2-(2-(2-(2-(4-((((S)-8-((5-(((S)-7-methoxy-2-methyl-5-oxo-5,11a-dihydro-1H-benzo[e]pyrrolo[1,2-a][1,4]diazepin-8-yl)oxy)pentyl)oxy)-2-methyl-5-oxo-5,11a-dihydro-1H-benzo[e]pyrrolo[1,2-a][1,4]diazepin-7-yl)oxy)methyl)-1H-1,2,3-triazol-1-yl)ethoxy)ethoxy)ethoxy)ethyl)propanamide,
1-(3-(2,5-dioxo-2,5-dihydro-1H-pyrrol-1-yl) propanamido)-N—((S)-1-(((S)-1-((4-((S)-7-methoxy-8-((5-(((S)-7-methoxy-2-(4-(4-methylpiperazin-1-yl)phenyl)-5-oxo-5,11a-dihydro-1H-benzo[e]pyrrolo[1,2-a][1,4]diazepin-8-yl)oxy)pentyl)oxy)-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl)-3,6,9,12,15,18,21,24-octaoxaheptacosan-27-amide,
6-(2,5-dioxo-2,5-dihydro-1H-pyrrol-1-yl)-N—((S)-1-(((S)-1-((4-((S)-8-(3-(((S)-2-(3-fluoro-4-methoxyphenyl)-7-methoxy-5-oxo-5,11a-dihydro-1H-benzo[e]pyrrolo[1,2-a][1,4]diazepin-8-yl)oxy) propoxy)-7-methoxy-5-oxo-5,11a-dihydro-1H-benzo[e]pyrrolo[1,2-a][1,4]diazepin-2-yl)phenyl)amino)-1-oxopropan-2-yl)amino)-3-methyl-1-oxobutan-2-yl) hexanamide,
(R)-2-((3-Nitropyridin-2-yl)disulfanyl)propyl(11S,11aS)-11-hydroxy-7-methoxy-8-(3-(((S)-7-methoxy-2-methylene-5-oxo-2,3,5,11a-tetrahydro-1H-pyrrolo[2,1-c][1,4]benzodiazepin-8-yl)oxy) propoxy)-2-methylene-5-oxo-2,3,11,11a-tetrahydro-1Hpyrrolo[2,1-c][1,4]benzodiazepine-10(5H)-carboxylate,
4-((2S,5S)-37-(2,5-dioxo-2,5-dihydro-1H-pyrrol-1yl)-5-isopropyl-2-methyl-4,7,35-trioxo-10,13,16,19,22,25,28,31-octaoxa-3,6,34-triazaheptatriacontanamido)benzyl(11S,11aS)-11-hydroxy-7-methoxy-8-(3-(((S)-7-methoxy-2-methylene-5-oxo-2,3,511a-tetrahydro-1H-benzo[e]pyrrolo[1,2-a][1,4]diazepin-8-yl)oxy) propoxy)-2-methylene-5-oxo-2,3,11,11a-tetrahydro-1H-benzo[e]pyrrolo[1,2-a][1,4]diazepine-10 (5H)-carboxylate, and
4-((2S,5S)-37-(2,5-dioxo-2,5-dihydro-1H-pyrrol-1-yl)-5-isopropyl-2-methyl-4,7,35-trioxo-10,13,16,19,22,25,28,31-octaoxa-3,6,34-triazaheptatriacontanamido)benzyl(11S,11aS)-11-hydroxy-8-(3-(((11S,11aS)-11-hydroxy-10-(((4-((10S,13S)-10-isopropyl-13-methyl-8,11-dioxo-2,5-dioxa-9,12-diazatetradecan-14-amido)benzyl)oxy) carbonyl)-7-methoxy-2-methylene-5-oxo-2,3,5,10,11,11a-hexahydro-1H-pyrrolo[2,1-c][1,4]benzodiazepin-8-yl)oxy) propoxy)-7-methoxy-2-methylene-5-oxo-2,3,11,11a-tetrahydro-1H-pyrrolo[2,1-c][1,4]benzodiazepine-10 (5H)-carboxylate. In these embodiments, the label is preferably a medicament for cancer.

In embodiments, a label is an octadentate thorium chelator such as described in WO2017211809A1. In these embodiments, the label is preferably a medicament for cancer.

In embodiments, a label is a dolastatin or an auristatin as described in WO2015162293A1. In these embodiments, the label is preferably a medicament for cancer.

In embodiments, a label is cytolysin or a Nigrin-b A-chain such as described in WO2015118030A2. In these embodiments, the label is preferably a medicament for cancer.

In embodiments, a label is 2-propylthiazolo[4,5-c]quinolin-4-amine, 1-(2-methylpropyl)-1H-imidazo[4,5-c]quinolin-4-amine, 4-amino-2-(ethoxymethyl)-α,α-di-methyl-1H-imidazo[4,5-c]quinoline-1-ethanol, 1-(4-amino-2-ethylaminomethylimidazo-[4,5-c]quinolin-1-yl)-2-methylpropan-2-ol, N-[4-(4-amino-2-ethyl-1H-imidazo[4,5-c]quinolin-1-yl)butyl-]methanesulfonamide, 4-amino-2-ethoxymethyl-aa-dimethyl-6,7,8,9-tetrahydro-1h-imidazo[4,5-c]quinoline-1-ethanol, 4-amino-aa-dimethyl-2-methoxyethyl-1h-imidazo[4,5-c]quinoline-1-ethanol, 1-{2-[3-(benzyloxy) propoxy]ethyl}-2-(ethoxymethyl)-1H-imidazo[4,5-c]quinolin-4-amine, N-[4-(4-amino-2-butyl-1H-imidazo[4,5-c][1,5]naphthyridin-1-yl)butyl]-n′-butylurea, N1-[2-(4-amino-2-butyl-1H-imidazo[4,5-c][1,5]naphthyridin-1-yl)ethyl]-2-amino-4-methylpentanamide, N-(2-{2-[4-amino-2-(2-methoxyethyl)-1H-imidazo[4,5-c]quinolin-1-yl]ethoxy}ethyl)-n′-phenylurea, 1-(2-amino-2-methylpropyl)-2-(ethoxymethyl)-1H-imidazo[4,5-c]quinolin-4-amine, 1-{4-[(3,5-dichlorophenyl) sulfonyl]butyl}-2-ethyl-1H-imidazo[4,5-c]quinolin-4-amine, N-(2-{2-[4-amino-2-(ethoxymethyl)-1H-imidazo[4,5-c]quinolin-1-yl]ethoxy}ethyl)-N′-cyclohexylurea, N-{3-[4-amino-2-(ethoxymethyl)-1H-imidazo[4,5-c]quinolin-1-yl]propyl}-n′-(3-cyanophenyl)thiourea, N-[3-(4-amino-2-butyl-1H-imidazo[4,5-c]quinolin-1-yl)-2,2-dimethylpropyl]benzamide, 2-butyl-1-[3-(methylsulfonyl) propyl]-1H-imidazo[4,5-c]quinolin-4-amine, N-{2-[4-amino-2-(ethoxymethyl)-1H-imidazo[4,5-c]quinolin-1-yl]-1.1-dimethylethyl}-2-ethoxyacetamide, 1-[4-amino-2-ethoxymethyl-7-(pyridin-4-yl)-1H-imidazo[4,5-c]quinolin-1-yl]-2-methylpropan-2-ol, 1-[4-amino-2-(ethoxymethyl)-7-(pyridin-3-yl)-1H-imidazo[4,5-c]quinolin-1-yl]-2-methylpropan-2-ol, N-{3-[4-amino-1-(2-hydroxy-2-methylpropyl)-2-(methoxyethyl)-1H-imidazo[4,5-c]quinolin-7-yl]phenyl}methanesulfonamide, 1-[4-amino-7-(5-hydroxymethylpyridin-3-yl)-2-(2-methoxyethyl)-1H-imidazo[4,5-c]quinolin-1-yl]-2-methylpropan-2-ol, 3-[4-amino-2-(ethoxymethyl)-7-(pyridin-3-yl)-1H-imidazo[4,5-c]quinolin-1-yl]propane-1,2-diol, 1-[2-(4-amino-2-ethoxymethyl-1H-imidazo[4,5-c]quinolin-1-yl)-1,1-dimethylethyl]-3-propylurea, 1-[2-(4-amino-2-ethoxymethyl-1H-imidazo[4,5-c]quinolin-1-yl)-1,1-dimethylethyl]-3-cyclopentylurea, 1-[(2,2-dimethyl-1,3-dioxolan-4-yl)methyl]-2-(ethoxymethyl)-7-(4-hydroxymethylphenyl)-1H-imidazo[4,5-c]quinolin-4-amine, 4-[4-amino-2-ethoxymethyl-1-(2-hydroxy-2-methylpropyl)-1H-imidazo[4,5-c]quinolin-7-yl]-N-methoxy-N-methylbenzamide, 2-ethoxymethyl-N1-isopropyl-6,7,8,9-tetrahydro-1H-imidazo[4,5-c]quinoline-1,4-diamine, 1-[4-amino-2-ethyl-7-(pyridin-4-yl)-1H-imidazo[4,5-c]quinolin-1-yl]-2-methylpropan-2-ol, N-[4-(4-amino-2-ethyl-1H-imidazo[4,5-c]quinolin-1-yl)butyl]methanesulfonamide, or N-[4-(4-amino-2-butyl-1H-imidazo[4,5-c][1,5]naphthyridin-1-yl)butyl]-N′-cyclohexylurea such as described in WO2015103990A1. In these embodiments, the label is preferably a medicament for cancer.

In embodiments, a label is a Pseudomonas exotoxin such as described in WO2015051199A2. In these embodiments, the label is preferably a medicament for cancer.

WO2017137553A1, WO2016038383A1, WO2018192944A1, WO2015051199A2, WO2017211809A1, WO2014057074A1, WO2015052322A1, WO2014140174A1, WO2015052321A1, WO2017186894A1, WO2017137555A1, WO2015162293A1, WO2015118030A2, WO2015103990A1, WO2017077382A1, WO2017134301A1, WO2017194783A1, WO2017194782A2, WO2018077893A1, WO2018141964A1, WO2018144999A1, WO2019032661A1, WO2019032663A1, WO2019032662A1, WO2019148089A1, WO2019191519A1 and WO2020033646A1 are incorporated in their entirety, and all compounds disclosed therein may be a label in the context of the current application.

In embodiments, a label is a prodrug of adrenomedullin as described in WO2013064508A1, an autotaxin inhibitor as described in WO2014097151A2, a pyrimido[4,5-b]quinoline-4,5(3h,10h)-dione derivative as described in WO2014091446A1, an amiloride derivative as described in WO2013064450A1, a pyrrolo[2,3-d]pyrimidine derivative as described in WO2014177527A1, a pyrazolopyridine derivative or a pyrazolopyrimidine derivative as described in WO2015173683A1, a piperidino-dihydrothienopyrimidine sulfoxide derivative as described in WO201326797A1, a 2-[pyridin-3-yl]-2,3-dihydro-benzo[1,4]dioxine derivative as described in WO2016061161A1, a pyridine derivative as described in WO201486705A1, a pyridine derivative or a pyrazine derivative as described in WO2011113894A1, an aminopyrimidinyl derivative as described in WO201627195A1, a carboxamide derivative as described in WO2015175796A1, an oxazole substituted indazole derivative as described in WO2010125082A1, a bisphenyl butanoic phosphonic acid derivative as described in WO2014126979A1, a pyrazine derivative as described in WO201235158A1, an oxazolidin-2-one-pyrimidine derivative as described in WO201472956A1, a pyrazolopyridinamine derivative as described in WO201696721A1, a N-(hetero)aryl, 2-(hetero) aryl-substituted acetamide derivative as described in WO2010101849A1, a 3-azabicyclo[3.1.0]hexane derivative as described in WO2017115205A1, a phenoxyacetamide derivative as described in WO201728927A1, a N-(5-(4-acetylpiperazin-I-yl)pyridin-2-yl)-2-(2′-fluoro-3-methyl-2,4′-bipyridin-5-yl) acetamide derivative or a 2-(2′,3-dimethyl-2,4′-bipyridin-5-yl)-N-(5-(pyrazin-2-yl) pyridin-2-yl) acetamide derivative as described in WO2017221142A1, a xanthine derivative as described in WO2013171166A1, a xanthine derivative as described in WO2013174768A1, a heterocyclylmethyl-thienouracile derivative as described in WO2016150901A1, an n,2-diarylquinoline-4-carboxamide derivative as described in WO201637954A1, a pyridin-2-amide derivative as described in WO2012168350A1, a benzamide derivative acting as modulator of cellular adhesion as described in WO2005044817A1, a 3-amino-pyridine derivative as described in WO2012117000A1, a Nampt or rock inhibitor as described in WO201267965A1, a oxetane derivative as described in WO201616242A1, a 1,1,1-trifluoro-3-hydroxypropan-2-yl carbamate derivative as described in WO2018134695A1, a pyrazol derivative as described in WO2014135507A1, an indole derivative as described in WO2009156462A1, a [1,2,3]triazolo[4,5-d]pyrimidine derivative as described in WO201815088A1, an sgc stimulator as described in WO2016177660A1, a CFTR protein as described in WO201760879A1, a 6-carboxylic acid of a benzimidazole or of a 4-aza-, 5-aza-, 7-aza- or 4,7-diaza-benzimidazole as described in WO2018109607A1, a benzamide derivative as described in WO201728926A1, an 8-azabicyclo[3.2.1]octane derivative as described in WO201287519A1, a triazolo[4,5-d]pyrimidine derivative as described in WO201671375A1, an aryl sultam derivative as described in WO2015104354A1, a 2-(azaindol-2-yl)benzimidazole derivative as described in WO201415905A1, or a quinuclidine or a iso-quinuclidine derivative as described in WO2005104745A1. Preferably the label is a medicament to treat fibrosis, wound healing, myocardial infarction, atherosclerosis, arthritis and/or other inflammatory and fibrotic diseases.

WO2013064508A1, WO2014097151A2, WO2014091446A1, WO2013064450A1, WO2014177527A1, WO2015173683A1, WO201326797A1, WO2016061161A1, WO201486705A1, WO2011113894A1, WO201627195A1, WO2015175796A1, WO2010125082A1, WO2014126979A1, WO201235158A1, WO201472956A1, WO201696721A1, WO2010101849A1, WO2017115205A1, WO201728927A1, WO2017221142A1, WO2013171166A1, WO2013174768A1, WO2016150901A1, WO201637954A1, WO2012168350A1, WO2005044817A1, WO2012117000A1, WO201267965A1, WO201616242A1, WO2018134695A1, WO2014135507A1, WO2009156462A1, WO201815088A1, WO2016177660A1, WO201760879A1, WO2018109607A1, WO201728926A1, WO201287519A1, WO201671375A1, WO2015104354A1, WO201415905A1 and WO2005104745A1 are incorporated in their entirety, and all compounds disclosed therein may be a label in the context of the current application.

In embodiments, a label is a molecule to be delivered to a cell, a tissue, an organ expressing human FOLR1. Any moiety, molecule or medicament known to act on a cell, tissue, organ expressing FOLR1 is potentially encompassed by these embodiments.

In embodiments, a label is a molecule to be delivered to the central nervous system (CNS). More preferably, the molecule is a medicament acting in the CNS, preferably acting in the brain. Even more preferably, the molecule crosses the brain blood barrier (BBB) and/or the blood-cerebrospinal fluid barrier (BCSFB) via transport via the human FOLR1; a mechanism called receptor mediated transcytosis (RMT). Any moiety, molecule or medicament known to act in the CNS or in the brain is potentially a label in the context of the present invention. Such a moiety, molecule or medicament may be any molecule or medicament, which does not cross the brain blood barrier (BBB) on its own. The molecule may be a peptide, a small molecule or a nucleic acid. A peptide may be a cytokine. A small molecule may be a chemotherapeutic. An entity may be a cell such as a CAR-T cell, a CAR-NK cell, a BITE or a LITE.

In embodiments, a medicament acting in the brain is a medicament for preventing and/or treating choroid plexus papilloma and/or hydrocephalus. Folate receptor alpha (FOLR1) is overexpressed in some human cancers, including choroid plexus papilloma or tumour. An increased expression of folate receptor alpha (FOLR1) in the brain has been associated with hydrocephalus.

In embodiments, a label is an aziridinyl-epothilone such as described in WO2007140297A2, preferably a label is 7,11-dihydroxy-17-[2-hydroxyethyl]-8,8,10,12-tetramethyl-3-[1-methyl-2-(2-methyl-4-thiazolyl) ethenyl]-4-oxa-17-azabicyclo[14.1.0]heptadecane-5,9-dione, or a derivative thereof, or a pharmaceutically acceptable salt thereof, more preferably said label is a compound for the treatment and/or prevention of choroid plexus papilloma, most preferably said label is comprised in a combination according to the invention for the treatment and/or prevention of choroid plexus papilloma.

In embodiments, a label is a 1H-pyrrolo[3,2-b]pyridine derivative such as described in WO2014145051A1, preferably a label is 4-(6-(3,5-dimethylisoxazol-4-yl)-1-(1-(pyridin-2-yl)ethyl)-1H-pyrrolo[3,2-b]pyridin-3-yl)benzoic acid, or 4-(6-(3,5-dimethylisoxazol-4-yl)-1-(phenyl(pyridin-2-yl)methyl)-1H-pyrrolo[3,2-b]pyridin-3-yl)benzoic acid, or methyl 2-(4-(6-(3,5-dimethylisoxazol-4-yl)-1-(1-(pyridin-2-yl)ethyl)-1H-pyrrolo[3,2-b]pyridin-3-yl)phenyl)acetate, or methyl 4-(6-(3,5-dimethylisoxazol-4-yl)-1-(pyridazin-3-ylmethyl)-1H-pyrrolo[3,2-b]pyridin-3-yl)benzoate, or N-cyclopropyl-4-(6-(3,5-dimethylisoxazol-4-yl)-1-(1-(pyridin-2-yl)ethyl)-1H-pyrrolo[3,2-b]pyridin-3-yl)benzenesulfonamide, or (4-(6-(3,5-dimethylisoxazol-4-yl)-1-(phenyl(pyridin-2-yl)methyl)-1H-pyrrolo[3,2-b]pyridin-3-yl)phenyl) methanol, or 3,5-dimethyl-4-(1-(pyridazin-3-ylmethyl)-3-(1-(2,2,2-trifluoroethyl)-1H-pyrazol-4-yl)-1H-pyrrolo[3,2-b]pyridin-6-yl) isoxazole, or 3,5-dimethyl-4-(3-(1-methyl-1H-pyrazol-4-yl)-1-(1-(pyridin-2-yl)ethyl)-1H-pyrrolo[3,2-b]pyridin-6-yl) isoxazole, or a derivative thereof, or a pharmaceutically acceptable salt thereof, more preferably said label is a compound for the treatment and/or prevention of choroid plexus papilloma, most preferably said label is comprised in a combination according to the invention for the treatment and/or prevention of choroid plexus papilloma. In an embodiment, a label is a 1H-pyrrolo[3,2-b]pyridine derivative such as described in WO2017053243A1, preferably a label is 4-(1-(1,1-di(pyridin-2-yl)ethyl)-6-(3,5-dimethylisoxazol-4-yl)-1H-pyrrolo[3,2-b]pyridin-3-yl)benzoic acid, or 4-(1-(cyanodipyridin-2-ylmethyl)-6-(3,5-dimethylisoxazol-4-yl)-1H-pyrrolo[3,2-b]pyridin-3-yl)benzoic acid, or 4-(6-(3,5-dimethylisoxazol-4-yl)-1-(fluorodipyridin-2-ylmethyl)-1H-pyrrolo[3,2-b]pyridin-3-yl)benzoic acid, or a derivative thereof, or a pharmaceutically acceptable salt thereof, more preferably said label is a compound for the treatment and/or prevention of choroid plexus papilloma, most preferably said label is comprised in a combination according to the invention for the treatment and/or prevention of choroid plexus papilloma.

In embodiments, a label is an pyrrolidine sulfonamide TRPC4 antagonists such as described in WO2018055524A1, preferably a label is 2-(((3R,4S)-4-(4-chlorophenoxy)-3-hydroxy-3-(hydroxymethyl) pyrrolidin-1-yl) sulfonyl)-5-(trifluoromethyl)benzonitrile, or 4-(((3S,4S)-1-((2-cyano-4-(trifluoromethyl)phenyl) sulfonyl)-4-hydroxy-4-((S)-1-hydroxyethyl) pyrrolidin-3-yl)oxy)-2-fluorobenzonitrile, or a derivative thereof, or a pharmaceutically acceptable salt thereof, more preferably said label is a compound for the treatment and/or prevention of hydrocephalus, most preferably said label is comprised in a combination according to the invention for the treatment and/or prevention of hydrocephalus.

In embodiments, a label is a sulfonpyrazole or sulfonylpyrazoline carboxamidine 5-HT6 antagonist such as described in WO2008034863A2, preferably a label is N′-(1-acetylindolin-5-ylsulfonyl)-N,4-diethyl-4,5-dihydro-1H-pyrazole-1-carboximidamide, or N′-(4-(1H-pyrazol-1-yl)phenylsulfonyl)-N,4-diethyl-4,5-dihydro-1H-pyrazole-1-carboximidamide, or N-((5-(N-((4-ethyl-4,5-dihydro-1H-pyrazol-1-yl) (ethylamino)methylene) sulfamoyl)thiophen-2-yl)methyl)benzamide, or N′-(5-(5-(chloromethyl)-1,2,4-oxadiazol-3-yl)thiophen-2-ylsulfonyl)-N,4-diethyl-4,5-dihydro-1H-pyrazole-1-carboximidamide, or a derivative thereof, or a pharmaceutically acceptable salt thereof, more preferably said label is a compound for the treatment and/or prevention of hydrocephalus, most preferably said label is comprised in a combination according to the invention for the treatment and/or prevention of hydrocephalus.

In embodiments, a label is an arylsulfonyl pyrazoline carboxamidine 5-HT6 antagonist such as described in WO2009115515A1, preferably a label is N′-(4-amino-3-chlorophenylsulfonyl)-N-ethyl-2,3,8-triazaspiro[4.5]dec-3-ene-2-carboximidamide, or N′-(4-amino-3-chlorophenylsulfonyl)-N-ethyl-2,3-diazaspiro[4.4]non-3-ene-2-carboximidamide, or N′-(4-amino-3-chlorophenylsulfonyl)-N-ethyl-4,4-dimethyl-4,5-dihydro-1H-pyrazole-1-carboximidamide, or N′-(4-aminophenylsulfonyl)-2,3-diazaspiro[4.4]non-3-ene-2-carboximidamide, or N′-(4-aminophenylsulfonyl)-8-oxa-2,3-diazaspiro[4.5]dec-3-ene-2-carboximidamide, or N′-(4-aminophenylsulfonyl)-N,4-diethyl-4,5-dihydro-1H-pyrazole-1-carboximidamide, or aminophenylsulfonyl)-N-ethyl-5-phenyl-4,5-dihydro-1H-pyrazole-1-carboximidamide, or N′-(4-aminophenylsulfonyl)-N-methyl-8-oxa-2,3-diazaspiro[4.5]dec-3-ene-2-carboximidamide, or a derivative thereof, or a pharmaceutically acceptable salt thereof.

In an embodiment, a label is a cyclodextrin-API conjugate such as described in WO2013116200A1, more preferably said label is a compound for the treatment and/or prevention of hydrocephalus, most preferably said label is comprised in a combination according to the invention for the treatment and/or prevention of hydrocephalus.

In embodiments, a label is pyrrolidine sulfonamide TRPV4 antagonists such as described in WO2018055527A1, preferably a label is 4-(((3S,4R)-1-((2,4-dichlorophenyl) sulfonyl)-4-hydroxy-4-N′-(4-(hydroxymethyl) pyrrolidin-3-yl)methyl)-2-fluorobenzonitrile, or 4-(((3S,4R)-1-((2-chloro-4-(trifluoromethyl)phenyl) sulfonyl)-4-hydroxy-4-(hydroxymethyl) pyrrolidin-3-yl)methyl)benzonitrile, or 4-(((3S,4R)-1-((5-chloropyridin-2-yl) sulfonyl)-4-hydroxy-4-(hydroxymethyl) pyrrolidin-3-yl)methyl)-3-(2,2,2-trifluoroethoxy)benzonitrile, or 4-(((3S,4S)-1-((2,4-dichlorophenyl) sulfonyl)-4-hydroxy-4-(hydroxymethyl) pyrrolidin-3-yl)methyl)-2-fluorobenzonitrile, or 4-(((3S,4R)-1-((2-cyano-4-(trifluoromethyl)phenyl) sulfonyl)-4-hydroxy-4-(hydroxymethyl) pyrrolidin-3-yl)methyl)-2-fluorobenzonitrile, or 4-(((3S,4R)-1-((2-chloro-4-cyanophenyl) sulfonyl)-4-hydroxy-4-(hydroxymethyl) pyrrolidin-3-yl)methyl)-2-fluorobenzonitrile, or 4-(((3S,4S)-4-(aminomethyl)-1-((5-chloropyridin-2-yl) sulfonyl)-4-hydroxypyrrolidin-3-yl)methyl)-2-fluorobenzonitrile, or a derivative thereof, or a pharmaceutically acceptable salt thereof, more preferably said label is a compound for the treatment and/or prevention of hydrocephalus, most preferably said label is comprised in a combination according to the invention for the treatment and/or prevention of hydrocephalus.

In embodiments, a label is [5-(5-chloro-1H-pyrrolo[2,3-b]pyridin-3-ylmethyl)-pyridin-2-yl]-(6-trifluoromethyl-pyridin-3-ylmethyl)-amine such as described in WO2016179415A1, or a derivative thereof, or a pharmaceutically acceptable salt thereof, preferably said label is a compound for the treatment and/or prevention of hydrocephalus, most preferably said label is comprised in a combination according to the invention for the treatment and/or prevention of hydrocephalus.

In embodiments, a label is a N-(pyridin-3-ylmethyl) substituted 1H-pyrrolo[2,3-b]pyridine derivative such as described in WO2013142427A1 or a 1H-pyrrolo[2,3-b]pyridine derivatives such as described in WO2017100201A1, or a derivative thereof, or a pharmaceutically acceptable salt thereof, preferably said label is a compound for the treatment and/or prevention of hydrocephalus, most preferably said label is comprised in a combination according to the invention for the treatment and/or prevention of hydrocephalus. WO2007140297A2, WO2014145051A1, WO2017053243A1, WO2018055524A1, WO2008034863A2, WO2009115515A1, WO2013116200A1, WO2018055527A1, WO2016179415A1, WO2013142427A1, and WO2017100201A1 are incorporated in their entirety, and all compounds disclosed therein may be a label in the context of the current application.

### Linkers

A combination according to the invention comprises a labelled compound. Below, preferred labelled compounds are provided. It is understood that these preferences imply preferred corresponding combinations according to the invention.

In embodiments, a labelled compound can be represented by CL-L¹-L²-R*, wherein CL is the click group comprised in the labelled compound, L¹ is a first linker or a bond, L² is a second linker, and R* is the label comprised in the labelled compound. Preferably, R* is a radionuclide as discussed above.

In embodiments, L¹ is a bond and the labelled compound can thus be represented by CL-L²-R*.

In embodiments, L¹ is a linker. Any suitable linker known to those skilled in the art in view of the present disclosure can be used in the invention. The linker can be, for example, a substituted or unsubstituted alkyl, a substituted or unsubstituted heteroalkyl moiety, a polyethylene glycol (PEG) linker, a hydrazone, a mal-caproyl polymer, a glucuronide (polymer), succinimide-thioethers, a peptide linker (such as a dipeptide linker), a sugar-based linker, or a cleavable linker, such as a disulfide linkage or a protease cleavage site such as a valine-citrulline, a valine-citrulline-PAB or a PAB polymer. In this context, the term polymer may be interpreted as an oligomer.

In embodiments, L¹ is an oligomer having 1, 2, 3, 4, 5, 6, 7, 8, 9, 10, 11, 12, 13, 14, 15, 16, 17, 18, 19 or 20 residues. In embodiments, L¹ is an oligomer having from 1 to 2, 3, 4, 5, 6, 7, 8, 9, 10, 11, 12, 13, 14, 15, 16, 17, 18, 19 or 20 residues. In embodiments, L¹ is an oligomer having from 5 to 6, 7, 8, 9, 10, 11, 12, 13, 14, 15, 16, 17, 18, 19 or 20 residues. In these embodiments, R* is preferably iodine-131, actinium-225, bismuth-213 lutetium-177, or terbium-161, more preferably iodine-131, actinium-225 or lutetium-177. In this context, an oligomer having 1 residue is a monomer.

In embodiments, L¹ is PEGₙ, wherein n is an integer from 1 to 2, 3, 4, 5, 6, 7, 8, 9, 10, 11, 12, 13, 14, 15, 16, 17, 18, 19 or 20. In embodiments, L¹ is PEGₙ, wherein n is an integer from 5 to 6, 7, 8, 9, 10, 11, 12, 13, 14, 15, 16, 17, 18, 19 or 20. In embodiments, L¹ is PEGₙ, wherein n is an integer from 6 to 7, 8, 9, 10, 11, 12, 13, 14, 15, 16, 17, 18, 19 or 20. In embodiments, L¹ is PEGₙ, wherein n is 1, 2, 3, 4, 5, 6, 7, 8, 9, 10, 11, 12, 13, 14, 15, 16, 17, 18, 19 or 20, most preferably wherein L¹ is PEG₇. In these embodiments, R* is preferably iodine-131, actinium-225, bismuth-213, lutetium-177, or terbium-161, more preferably iodine-131, actinium-225 or lutetium-177.

The type of the second linker L² depends on the identity of R*, particularly is R* is a radionuclide as discussed above. Without being limiting, the radioactive isotopes of iodine (preferred R*) possess the ability to be directly integrated into a molecule by electrophilic or nucleophilic substitution or indirectly via conjugation, implying a covalent bond between L² and R*. Radioactive metals on the other hand are labelled via complexation with a chelator, implying that the bond between L² and R* is non-covalent.

In embodiments, L² is a chelator, preferably wherein the bond L²-R* is a non-covalent, coordinative bond. In embodiments, L² is a chelator and R* is a radionuclide chosen from the group consisting of non-covalently attached radionuclides, preferably actinium-225, bismuth-212, bismuth-213, caesium-137, chromium-51, cobalt-60, copper-67, erbium-169, fermium-255, gold-198, iron-59, lead-212, lutetium-177, holium-166, potassium-42, rhenium-186, rhenium-188, samarium-153, radium-223, radium-224, scandium-47, sodium-24, terbium-149, or terbium-161, more preferably actinium-225, bismuth-212, bismuth-213, copper-67, erbium-169, iron-59, lead-212, lutetium-177, samarium-153, radium-223, radium-224, scandium-47, terbium-149, terbium-161, thorium-227, ytterbium-169, ytterbium-177, or yttrium-90, most preferably wherein R* is actinium-225, bismuth-213, lutetium-177 or terbium-161, most preferably actinium-225 or lutetium-177.

As used herein, the term “chelator” refers to a chemical group to which a metal (such as actinium-225 or lutetium-177) can be chelated via coordinate bonding. Any chelator known to those skilled in the art in view of the present disclosure can be used in the invention.

In embodiments, a chelator comprises a macrocycle. Preferred chelators comprising a macrocycle include, but are not limited to, 1,4,7,10-tetraazacyclododecane-N,N′,N″,N″-tetraacetic acid (DOTA), 1,4,7,10,13,16-hexaazacyclohexadecane-N,N′,N″,N″,N″,N″″-hexaacetic acid (HEHA), 1,4,7,10,13-pentaazacyclopentanadecane-N,N′,N″,N″,N″-pentaacetic acid (PEPA), N,N′-bis[(6-carboxy-2-pyridil)methyl]-4.13-diaza-18-crown-6 (Macropa) (Thiele et al., An Eighteen-Membered Macrocyclic Ligand for Actinium-225 Targeted Alpha Therapy. Angew Chem Int Ed Engl. 2017 Nov. 13; 56 (46): p. 14712-14717), 1,4,8,11-tetraazacyclotetradecane-1,4,8,11-tetraacetic acid (TETA), 1,4,7,10-tetraazacyclododecane-1,4,7,10-tetrapropionic acid (DOTPA), 1,4,8,11-tetraazacyclotetradecane-1,4,8,11-tetrapropionic acid (TETPA), 1,4,7,10-tetraazacyclododecane-1,4,7,10-tetramethylenephosphonic acid (DOTMP), 3,6,9,15-tetraazabicyclo[9.3.1]pentadeca-1 (15), 11.13-triene-3,6,9-triacetic acid (PCTA), and 2,2′,2″,2′-(1,10-dioxa-4,7,13,16-tetraazacyclooctadecane-4,7,13,16-tetrayl)tetraacetic acid (Crown) (Yang, Hua, et al. “Synthesis and Evaluation of a Macrocyclic Actinium-225 Chelator, Quality Control and In Vivo Evaluation of 225Ac-crown-αMSH Peptide.” Chemistry—A European Journal 26.50 (2020): 11435-11440). Preferably, a chelator is 1,4,7,10-tetraazacyclododecane-1,4,7,10-tetraacetic acid (DOTA), 1,4,7-triazacyclononane-1,4,7-triacetic acid (NOTA), Macropa, PCTA or Crown.

In embodiments, a labelled compound can be represented by ²²⁵Ac-DOTA-PEGₙ-CL, ²²⁵Ac-HEHA-PEGₙ-CL, ²²⁵Ac-PEPA-PEGₙ-CL, ²²⁵Ac-Macropa-PEGₙ-CL, ²²⁵Ac-TETA-PEGₙ-CL, ²²⁵Ac-DOTPA-PEGₙ-CL, ²²⁵Ac-TETPA-PEGₙ-CL or ²²⁵Ac-DOTMP-PEGₙ-CL, ¹⁷⁷Lu-DOTA-PEGₙ-CL, ¹⁷⁷Lu-HEHA-PEGₙ-CL, ¹⁷⁷Lu-PEPA-PEGₙ-CL, ¹⁷⁷Lu-Macropa-PEGₙ-CL, ¹⁷⁷Lu-TETA-PEGₙ-CL, ¹⁷⁷Lu-DOTPA-PEGₙ-CL, ¹⁷⁷Lu-TETPA-PEGₙ-CL or ¹⁷⁷Lu-DOTMP-PEGₙ-CL, preferably wherein n is an integer from 1 to 2, 3, 4, 5, 6, 7, 8, 9, 10, 11, 12, 13, 14, 15, 16, 17, 18, 19 or 20; or wherein n is an integer from 5 to 6, 7, 8, 9, 10, 11, 12, 13, 14, 15, 16, 17, 18, 19 or 20; or wherein n is 1, 2, 3, 4, 5, 6, 7, 8, 9, 10, 11, 12, 13, 14, 15, 16, 17, 18, 19 or 20; most preferably wherein L¹ is PEG₇. Preferably, CL is a tetrazine (Tz).

In embodiments, a labelled compound can be represented by ²²⁵Ac-DOTA-PEG₇-Tz or ¹⁷⁷Lu-DOTA-PEG₇-Tz.

In embodiments, a chelator comprises an open chain ligand. Preferred chelators comprising an open chain ligand, but are not limited to, deferoxamine (DFO), ethylenediaminetetraacetic acid (EDTA), and diethylenetriaminepentaacetic acid (DTPA).

In embodiments, a labelled compound can be represented by ²²⁵Ac-DFO-PEGₙ-Tz, ²²⁵Ac-EDTA-PEGₙ-Tz, ²²⁵Ac-DTPA-PEGₙ-Tz, ¹⁷⁷Lu-DFO-PEGₙ-Tz, ¹⁷⁷Lu-EDTA-PEGₙ-Tz, or ¹⁷⁷Lu-DTPA-PEGₙ-Tz, preferably wherein n is an integer from 1 to 2, 3, 4, 5, 6, 7, 8, 9, 10, 11, 12, 13, 14, 15, 16, 17, 18, 19 or 20; or wherein n is an integer from 5 to 6, 7, 8, 9, 10, 11, 12, 13, 14, 15, 16, 17, 18, 19 or 20; or wherein n is 1, 2, 3, 4, 5, 6, 7, 8, 9, 10, 11, 12, 13, 14, 15, 16, 17, 18, 19 or 20; most preferably wherein L¹ is PEG₇. Preferably, CL is a tetrazine.

In embodiments, a labelled compound can be represented by ²²⁵Ac-EDTA-PEG₇-Tz or ¹⁷⁷Lu-EDTA-PEG₇-Tz.

In embodiments, L² is a group which is bound covalently to the label. In other words, L²-R* is a covalent bond. Preferably, R* is astatine-211, dysprosium-165, iodine-125, iodine-131, iridium-192, molybdenum-99, palladium-103, phosphorus-32, ruthenium-106, strontium-89, or xenon-133.

In embodiments, L² is a benzoate linker, preferably 3-pyridinecarboxyl, benzoate or guadinomethyl benzoate, even more preferably guadinomethyl benzoate, most preferably 4-guadinomethyl benzoate. These active esters exhibit a high in vivo stability.

In embodiments, a labelled compound can be represented by R*-L²-PEGₙ-CL, as defined above, wherein L² is a benzoate linker, more preferably 4-guadinomethyl benzoate, even more preferably wherein R* is iodine-131, most preferably wherein n is 7.

In embodiments, a labelled compound can be represented by [I-131]GMIB-PEGₙ-CL, as defined above, wherein [I-131]GMIB is 4-guanidinomethyl-3-[I-131]iodobenzoate, preferably wherein n is an integer from 1 to 2, 3, 4, 5, 6, 7, 8, 9, 10, 11, 12, 13, 14, 15, 16, 17, 18, 19 or 20; or wherein n is an integer from 5 to 6, 7, 8, 9, 10, 11, 12, 13, 14, 15, 16, 17, 18, 19 or 20; or wherein n is 1, 2, 3, 4, 5, 6, 7, 8, 9, 10, 11, 12, 13, 14, 15, 16, 17, 18, 19 or 20; most preferably wherein L¹ is PEG₇. It is understood that in such a labelled compound the linker L² is guadinomethyl benzoate and R* is iodine-131.

A labelled compound represented by [I-131]GMIB-PEGₙ-CL may also be represented by the following structure:

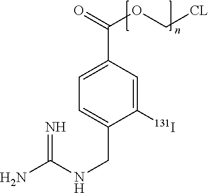

In embodiments, a labelled compound can be represented by [I-131]GMIB-CL, as defined above. Labelled compounds according to these embodiments may also be represented by the following structure:

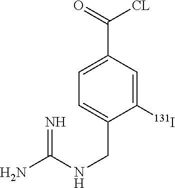

In embodiments, a labelled compound can be represented by [I-131]GMIB-PEGₙ-Tz, as defined above, wherein [I-131]GMIB is 4-guanidinomethyl-3-[I-131]iodobenzoate, preferably wherein n is an integer from 1 to 2, 3, 4, 5, 6, 7, 8, 9, 10, 11, 12, 13, 14, 15, 16, 17, 18, 19 or 20; or wherein n is an integer from 5 to 6, 7, 8, 9, 10, 11, 12, 13, 14, 15, 16, 17, 18, 19 or 20; or wherein n is 1, 2, 3, 4, 5, 6, 7, 8, 9, 10, 11, 12, 13, 14, 15, 16, 17, 18, 19 or 20; most preferably wherein L¹ is PEG₇. It is understood that in such a labelled compound the linker L² is guadinomethyl benzoate and R* is iodine-131.

In embodiments, a labelled compound can be represented by [I-131]GMIB-Tz, as defined above.

### Antigens

### General

In embodiments, a VHH, or a fragment thereof, according to the invention is able to specifically bind an antigen, wherein said antigen is comprised in a target, wherein said binding of said VHH, or fragment thereof, to said antigen does cause a significant conformational change in said target.

In embodiments, a VHH, or a fragment thereof, according to the invention is able to specifically bind an antigen with a dissociation constant (k_{off}) equal to or smaller than 10⁻¹ s⁻¹, preferably equal to or smaller than 10⁻² s⁻¹, more preferably equal to or smaller than 10⁻³ s⁻¹, most preferably equal to or smaller than 10⁻⁴ s⁻¹. In embodiments, a VHH, or a fragment thereof, according to the invention is able to specifically bind an antigen with a dissociation constant (K_{off}) in the range from 10⁻¹ s⁻¹ to 10⁻⁵ s⁻¹, preferably in the range from 10⁻² s⁻¹ to 10⁻⁵ s⁻¹, more preferably in the range from 10⁻³ s⁻¹ to 10⁻⁵ s⁻¹, most preferably in the range from 10⁻⁴ s⁻¹ to 10⁻⁵ s⁻¹.

In embodiments, a VHH, or a fragment thereof, according to the invention is able to specifically bind an antigen, wherein said antigen is present on the surface of a tumour cell or in a tumour microenvironment, more preferably with a dissociation constant (K_{off}) equal to or smaller than 10⁻¹ s⁻¹, preferably equal to or smaller than 10⁻² s⁻¹, more preferably equal to or smaller than 10⁻³ s⁻¹, most preferably equal to or smaller than 10⁻⁴ s⁻¹.

In embodiments, a VHH, or a fragment thereof, according to the invention is able to specifically bind an antigen, wherein said antigen is present on the surface of a tumour cell or in a tumour microenvironment, more preferably with a dissociation constant (K_{off}) in the range from 10⁻¹ s⁻¹ to 10⁻⁵ s⁻¹, preferably in the range from 10⁻² s⁻¹ to 10⁻⁵ s⁻¹, more preferably in the range from 10⁻³ s⁻¹ to 10⁻⁵ s⁻¹, most preferably in the range from 10⁻⁴ s⁻¹ to 10⁻⁵ s⁻¹.

In embodiments, a VHH, or a fragment thereof, according to the invention is able to specifically bind an antigen, wherein said antigen is an oncogene and/or wherein said antigen is expressed on a cancer cell or in a tumour microenvironment, more preferably with a dissociation constant (K_{off}) equal to or smaller than 10⁻¹ s⁻¹, preferably equal to or smaller than 10⁻² s⁻¹, more preferably equal to or smaller than 10⁻³ s⁻¹, most preferably equal to or smaller than 10⁻⁴ s⁻¹.

In embodiments, a VHH, or a fragment thereof, according to the invention is able to specifically bind an antigen, wherein said antigen is an oncogene and/or wherein said antigen is expressed on a cancer cell or in a tumour microenvironment, more preferably with a dissociation constant (K_{off}) in the range from 10⁻¹ s⁻¹ to 10⁻⁵ s⁻¹, preferably in the range from 10⁻² s⁻¹ to 10⁻⁵ s⁻¹, more preferably in the range from 10⁻³ s⁻¹ to 10⁻⁵ s⁻¹, most preferably in the range from 10⁻⁴ s⁻¹ to 10⁻⁵ s⁻¹.

In embodiments, said antigen is CEA, PSMA, 4lg-B7-H3, A33, α5ß1 Integrin, AGS-16, AGS-8, αvß3 Integrin, CAIX/MN, CCR-2/CCL-2, CD40, CD44v6, CK19, CTLA-4, d9-E-cadherin, DNA/histone H1, ED-B, fibronectin, EFGL7, EGFR, EGP-1, Endoglin, EpCAM, FAP, FZD10, GC128, GD2, GD3, HER-2, HER-3, HPV-16 E6, IGF-1R, IL-6, L1-CAM, Lewisy, MAGE-A3, Melanin, Mesothelin, MET/HGF, MUC1, NCAM, Neuropilin-1, PD-1/PD-L1, PDGFR, PSCA, TAG-72, TEM-1, Tenascin-C, Thrombomodulin, TRAIL-R, Tweak-R, VE cadherin, VEGFR/VEGF, or VEGFR1. Preferably, said antigen is an oncogene and/or is expressed on a cancer cell or in a tumour microenvironment.

In embodiments, a VHH, or a fragment thereof, according to the invention is able to specifically bind human fibroblast activation protein (FAP), human folate receptor alpha (FOLR1) or human epidermal growth factor receptor 2 (HER2), preferably human fibroblast activation protein (FAP) or human folate receptor alpha (FOLR1).

### Fibroblast Activation Protein (FAP)

In embodiments, a VHH, or a fragment thereof, according to the invention is particularly suited for binding to human and/or murine FAP. In an embodiment, the VHH, or the fragment thereof, specifically binds human and murine FAP. In an embodiment, the VHH, or the fragment thereof, binds part of the extracellular domain of human and/or murine FAP.

Examples 3, 5 and 6 show the usefulness of the pretargeting approach of FAP-bindings VHHs for treating cancer. Example 3 presents the long-term biodistribution and tumor targeting of ¹⁷⁷Lu-VHH1 using a pre-targeting strategy. Example 5 demonstrates the therapeutic potential of pre-targeted ²²⁵Ac-VHH1 in tumor xenografted mouse models without any relevant signs of acute toxicity. Example 6 investigates the long-term toxicology of healthy female C57BI/6 mice treated with pre-targeted ²²⁵Ac-VHH1, revealing no signs of acute or long-term toxicity.

A VHH, or a fragment thereof, according to the invention and a combination according to the invention are preferably used for treating a cancer associated with the expression of human FAP in a CAF cell and/or in a cancer cell.

In another application, the moiety may be a medicament to treat fibrosis, wound healing, myocardial infarction, atherosclerosis, arthritis and other inflammatory and fibrotic diseases. In such applications, the antibody fragment of the invention is used to target the medicament to the site of the disease listed in order to treat it.

Human FAP is quite attractive to be targeted as it is specifically expressed and more specifically overexpressed in cancer-associated fibroblasts (CAF) which have a tumourigenic function (Puré et al 2018, Oncogene August; 37 (32): 4343-4357). It is also expressed in some cancer cells (such as leukemia, bone, uterus, pancreas, skin, muscle, brain, breast, colorectal, oesophageal, gastric, liver, lung, ovarian, parathyroid, renal cancer as disclosed later herein) and poorly expressed in healthy cells. Human FAP may therefore be considered as a tumour antigen or a cancer cell antigen and may therefore be used as diagnostic and/or therapeutic target.

However other applications (diagnostic and therapeutic) of the VHH, or the fragment thereof, of the invention are also encompassed by the present invention. Such other diagnostic and/or therapeutic applications are not linked to cancer but may be linked to fibrosis, wound healing, myocardial infarction, atherosclerosis, arthritis and other inflammatory and fibrotic diseases. In other words, the VHH, or the fragment thereof of the invention may be used in a diagnostic and/or therapeutic application to diagnose and/or treat fibrosis, wound healing, myocardial infarction, atherosclerosis, arthritis and other inflammatory and fibrotic diseases. More detailed explanation is given later herein.

As used herein, human FAP is considered a ‘cancer cell-specific antigen’, ‘cancer-specific antigen’, ‘cancer antigen’, ‘target protein present on, ‘target protein expressed in’ and/or ‘specific for a cancer cell’, ‘cancer cell-specific target (protein)’, ‘cancer (cell)-associated antigen’ are used interchangeably herein and refers to the fact that human FAP is mainly present on (or mainly expressed on) cancer cells and in the vicinity of a tumour and/or in the vicinity of metastases. FAP is specifically expressed and more specifically overexpressed in cancer-associated fibroblasts (CAF) which have a tumourigenic function. It is also expressed in some cancer cells (such as leukemia, bone, uterus, pancreas, skin, muscle, brain, breast, colorectal, oesophageal, gastric, liver, lung, ovarian, parathyroid, renal cancer (Puré et al 2018, Oncogene August; 37 (32): 4343-4357, as disclosed later herein). FAP is poorly expressed in healthy cells. Human FAP may therefore be considered as a tumour antigen or a cancer cell antigen and may therefore be used as diagnostic and/or therapeutic target. For example, human FAP is expressed in cancer-associated fibroblast.

As used herein, the term “FAP positive” or “expressing FAP” or “overexpressing FAP” may refer to cancerous or malignant human cells and/or cancer-associated fibroblasts or tissue characterized by FAP protein overexpression and thus have abnormally high levels of the FAP gene and/or the FAP protein compared to normal healthy cells. In this context, “overexpressing” may mean that the expression is at least 10%, 20%, 30%, 40%, 50%, 60%, 70%, 80%, 90%, 100% or more than the expression in a control cell line. A control cell line may be a healthy or non-diseased cell.

In an embodiment, a VHH, or a fragment thereof, according to the invention, specifically binds human and/or murine FAP, and fulfils at least one of the following:
a. the epitope is comprised within amino acid 26 to 760 of SEQ ID NO: 5, preferably the epitope is comprised within (or comprises) amino acids 65-90 and/or 101-140 of SEQ ID NO: 5,
b. wherein at least amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO: 5 interacts with said VHH or fragment thereof,
c. said VHH or fragment thereof is represented by an amino acid sequence that comprises an amino acid sequence having at least 80% sequence identity with at least one of SEQ ID NO: 1, 2, 3, 4 or a portion thereof.

In an embodiment, a VHH, or a fragment thereof, according to the invention, specifically binds human and/or murine FAP, and fulfils at least one of the following:
a. the epitope is comprised within amino acids 26 to 760 of SEQ ID NO: 5, preferably the epitope is comprised within (or comprises) amino acids 65-90 and/or 101-140 of SEQ ID NO: 5,
b. the VHH, or the fragment thereof, specifically binds to the following amino acids of SEQ ID NO: 5:
   1) at least one, or at least two or at least three amino acids selected from: I62, S63, G64, Q65, E66, and/or
   2) at least one, or at least two or at least three amino acids selected from: I76, V77, L78, Y79, N80, I81, E82, T83, G84, Q85, S86, Y87, T88, I89, L90, S91, and/or
   3) at least one, or at least two or at least three amino acids selected from L105, S106, P107, D108, R109, Q110, F111, and/or
   4) at least one, or at least two or at least three amino acids selected from D134, L135, S136, N137, and/or
   5) V158 and/or G159 and/or
   6) R175, and/or
   7) D457 and/or Y458,
c. the VHH, or the fragment thereof, is represented by an amino acid sequence that comprises an amino acid sequence having at least 80% sequence identity with at least one of SEQ ID NO: 1, 2, 3, 4 or a portion thereof.

In an embodiment, a VHH, or a fragment thereof, according to the invention specifically binds human and/or murine FAP, and has its epitope comprised within amino acids 26 to 760 of SEQ ID NO: 5, specifically binds to the following amino acids of SEQ ID NO: 5:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

In an embodiment, a VHH, or a fragment thereof, according to the invention specifically binds human and/or murine FAP, and has its epitope comprised within (or has its epitope which comprises) amino acids 65-90 and/or 101-140 of SEQ ID NO: 5, specifically binds to the following amino acids of SEQ ID NO: 5:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

Throughout the application, FAP is synonymous with FAPalpha and corresponds to the polypeptide Prolyl endopeptidase FAP, which is also named Fibroblast activation protein alpha (FAPalpha). The gene encoding this protein is called FAP.

TABLE 1
Amino acid sequence of human and murine FAP
(derived from Uniprot entry Q12884 and P97321,
respectively)

| | |
|---|---|
| SEQ ID NO: 5 | MKTWVKIVFGVATSAVLALLVMCIVLRPSRVHNSE |
| Human | ENTMRALTLKDILNGTFSYKTFFPNWISGQEYLHQ |
| | SADNNIVLYNIETGQSYTILSNRTMKSVNASNYGL |
| | SPDRQFVYLESDYSKLWRYSYTATYYIYDLSNGEF |
| | VRGNELPRPIQYLCWSPVGSKLAYVYQNNIYLKQR |
| | PGDPPFQITFNGRENKIFNGIPDWVYEEEMLATKY |
| | ALWWSPNGKFLAYAEFNDTDIPVIAYSYYGDEQYP |
| | RTINIPYPKAGAKNPVVRIFIIDTTYPAYVGPQEV |
| | PVPAMIASSDYYFSWLTWVTDERVCLQWLKRVQNV |
| | SVLSICDFREDWQTWDCPKTQEHIEESRTGWAGGF |
| | FVSTPVFSYDAISYYKIFSDKDGYKHIHYIKDTVE |
| | NAIQITSGKWEAINIFRVTQDSLFYSSNEFEEYPG |
| | RRNIYRISIGSYPPSKKCVTCHLRKERCQYYTASF |
| | SDYAKYYALVCYGPGIPISTLHDGRTDQEIKILEE |
| | NKELENALKNIQLPKEEIKKLEVDEITLWYKMILP |
| | PQFDRSKKYPLLIQVYGGPCSQSVRSVFAVNWISY |
| | LASKEGMVIALVDGRGTAFQGDKLLYAVYRKLGVY |
| | EVEDQITAVRKFIEMGFIDEKRIAIWGWSYGGYVS |
| | SLALASGTGLFKCGIAVAPVSSWEYYASVYTERFM |
| | GLPTKDDNLEHYKNSTVMARAEYFRNVDYLLIHGT |
| | ADDNVHFQNSAQIAKALVNAQVDFQAMWYSDQNHG |
| | LSGLSTNHLYTHMTHFLKQCFSLSD |
| SEQ ID NO: 6 | MKTWLKTVFGVTTLAALALVVICIVLRPSRVYKPE |
| Murine | GNTKRALTLKDILNGTFSYKTYFPNWISEQEYLHQ |
| | SEDDNIVFYNIETRESYIILSNSTMKSVNATDYGL |
| | SPDRQFVYLESDYSKLWRYSYTATYYIYDLQNGEF |
| | VRGYELPRPIQYLCWSPVGSKLAYVYQNNIYLKQR |
| | PGDPPFQITYTGRENRIFNGIPDWVYEEEMLATKY |
| | ALWWSPDGKFLAYVEFNDSDIPIIAYSYYGDGQYP |
| | RTINIPYPKAGAKNPVVRVFIVDTTYPHHVGPMEV |
| | PVPEMIASSDYYFSWLTWVSSERVCLQWLKRVQNV |
| | SVLSICDFREDWHAWECPKNQEHVEESRTGWAGGF |
| | FVSTPAFSQDATSYYKIFSDKDGYKHIHYIKDTVE |
| | NAIQITSGKWEAIYIFRVTQDSLFYSSNEFEGYPG |
| | RRNIYRISIGNSPPSKKCVTCHLRKERCQYYTASF |
| | SYKAKYYALVCYGPGLPISTLHDGRTDQEIQVLEE |
| | NKELENSLRNIQLPKVEIKKLKDGGLTFWYKMILP |
| | PQFDRSKKYPLLIQVYGGPCSQSVKSVFAVNWITY |
| | LASKEGIVIALVDGRGTAFQGDKFLHAVYRKLGVY |
| | EVEDQLTAVRKFIEMGFIDEERIAIWGWSYGGYVS |
| | SLALASGTGLFKCGIAVAPVSSWEYYASIYSERFM |
| | GLPTKDDNLEHYKNSTVMARAEYFRNVDYLLIHGT |
| | ADDNVHFQNSAQIAKALVNAQVDFQAMWYSDQNHG |
| | ISSGRSQNHLYTHMTHFLKQCFSLSD |

The binding to human and/or murine FAP may be assessed in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold). 5

In an embodiment, the binding may be assessed in vitro using cells expressing human and/or murine FAP, and optionally in vivo or ex vivo as earlier defined herein. These cells may be human cells and expressing endogenous human and/or murine FAP. Alternatively, these cells may overexpress human and/or murine FAP. Cells overexpressing human and/or murine FAP may be human or non-human cells. Preferred cells are fibroblast cells expressing human and/or murine FAP. A preferred transfected cell line is HEK293. A 10 preferred cell line expressing FAP is GM05389 or U-87 MG. A preferred cancer cell line expressing human FAP is U-87 MG.

In an embodiment, a VHH, or a fragment thereof, according to the invention specifically binds to human and/or murine FAP. This assessment is preferably carried out using ELISA, Surface Plasmon Resonance or Bio-Layer Interferometry.

It is also expected that a VHH, or a fragment thereof, according to the invention will bind to a number of naturally occurring or synthetic analogues, variants, mutants, alleles, parts and fragments of human and/or murine FAP.

In an embodiment, a VHH, or a fragment thereof, according to the invention will specifically bind to at least those analogues, variants, mutants, alleles, naturally occurring, synthetic analogues, parts and fragments of human and/or murine FAP that (still) contain the epitope of the (natural/wild-type) antigen to which the VHH or fragment thereof binds.

In an embodiment, the epitope of human FAP of a VHH or fragment thereof according to the invention is comprised within amino acids 26 to 760 of SEQ ID NO: 5.

In an embodiment, the epitope of a VHH, or a fragment thereof, according to the invention is comprised within amino acids 65-90 and/or 101-140 of SEQ ID NO: 5.

In an embodiment, the epitope of a VHH, or a fragment thereof, according to the invention comprises the amino acid stretch or region 65-90 and/or 101-140 of SEQ ID NO: 5.

In an embodiment, the epitope of a VHH, or a fragment thereof, according to the invention comprises the combination of amino acid stretch or region 65-90 and 101-140 of SEQ ID NO: 5.

In an embodiment, the epitope of a VHH, or a fragment thereof, according to the invention is comprised within the combination of amino acid stretch or region 65-90 and 101-140 of SEQ ID NO: 5.

In an embodiment, at least one of the following amino acids of SEQ ID NO: 5 is bound or contacted or interacts with a VHH, or a fragment thereof, according to the invention: I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458.

In an embodiment, a VHH, or a fragment thereof, according to the invention specifically binds to the following amino acids of SEQ ID NO: 5:
1) at least one, or at least two or at least three amino acids selected from: I62, S63, G64, Q65, E66, and/or
2) at least one, or at least two or at least three amino acids selected from: I76, V77, L78, Y79, N80, I81, E82, T83, G84, Q85, S86, Y87, T88, I89, L90, S91, and/or
3) at least one, or at least two or at least three amino acids selected from L105, S106, P107, D108, R109, Q110, F111, and/or
4) at least one, or at least two or at least three amino acids selected from D134, L135, S136, N137, and/or
5) V158 and/or G159 and/or
6) R175, and/or
7) D457 and/or Y458.

In an embodiment, a VHH, or a fragment thereof, according to the invention specifically binds to the following amino acids of SEQ ID NO: 5:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

In an embodiment, a VHH, or a fragment thereof, according to the invention specifically binds to the following amino acids of SEQ ID NO: 5:
1) at least two or at least three amino acids selected from: I62, S63, G64, Q65, E66, and/or
2) at least two or at least three amino acids selected from: I76, V77, L78, Y79, N80, I81, E82, T83, G84, Q85, S86, Y87, T88, I89, L90, S91, and/or
3) at least two or at least three amino acids selected from L105, S106, P107, D108, R109, Q110, F111, and/or
4) at least two or at least three amino acids selected from D134, L135, S136, N137, and/or
5) V158 and G159 and/or
6) R175, and/or
7) D457 and Y458.

In an embodiment, a VHH, or a fragment thereof, according to the invention specifically binds to the following amino acids of SEQ ID NO: 5:
1) at least two or at least three amino acids selected from: I62, S63, G64, Q65, E66, and
2) at least two or at least three amino acids selected from: I76, V77, L78, Y79, N80, I81, E82, T83, G84, Q85, S86, Y87, T88, I89, L90, S91, and
3) at least two or at least three amino acids selected from L105, S106, P107, D108, R109, Q110, F111, and
4) at least two or at least three amino acids selected from D134, L135, S136, N137, and
5) V158 and G159 and
6) R175, and
7) D457 and Y458.

In an embodiment, a VHH, or a fragment thereof, according to the invention specifically binds to the following amino acids of SEQ ID NO: 5:
1) at least three amino acids selected from: I62, S63, G64, Q65, E66, and/or
2) at least three amino acids selected from: I76, V77, L78, Y79, N80, I81, E82, T83, G84, Q85, S86, Y87, T88, 189, L90, S91, and/or
3) at least three amino acids selected from L105, S106, P107, D108, R109, Q110, F111, and/or
4) at least three amino acids selected from D134, L135, S136, N137, and/or
5) V158 and G159 and/or
6) R175, and
7) D457 and Y458.

In an embodiment, a VHH, or a fragment thereof, according to the invention specifically binds to the following amino acids of SEQ ID NO: 5:
1) at least three amino acids selected from: I62, S63, G64, Q65, E66, and
2) at least three amino acids selected from: I76, V77, L78, Y79, N80, I81, E82, T83, G84, Q85, S86, Y87, T88, I89, L90, S91, and
3) at least three amino acids selected from L105, S106, P107, D108, R109, Q110, F111, and
4) at least three amino acids selected from D134, L135, S136, N137, and
5) V158 and G159 and
6) R175, and
7) D457 and Y458.

In an embodiment, the following amino acids of SEQ ID NO: 5 are bound or contacted or interacted with a VHH, or a fragment thereof, according to the invention: I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458. In this context, an amino acid of human of FAP may be bound by the VHH, or the fragment thereof, when said amino acid belongs to the epitope of the VHH, or the fragment thereof.

A VHH, or a fragment thereof, according to the invention is said to be ‘specific’ for a first target antigen of interest (i.e. human and/or murine FAP) as opposed to a second molecule, such as one of the closest homologues of FAP (i.e. DPP IV, Dipeptidyl amino-peptidase IV, Juillerat-Jeanneret, L., et al (2017), Expert Opinion on therapeutic targets, 21:977-991) when it binds to the first target antigen of interest with an affinity that is at least 5 times, such as at least 10 times, such as at least 100 times, and preferably at least 1000 times higher than the affinity with which that VHH, or the fragment thereof, binds to the second molecule. The amino acid sequence of DPP IV has 52% identity with the amino acid sequence of FAP and still the VHH, or the fragment thereof, can distinguish between the two related prolyl-specific serine proteases. Accordingly, in certain embodiments, when a VHH, or a fragment thereof, according to the invention is said to be ‘specific for’ a first target antigen of interest as opposed to a second molecule, it may specifically bind to (as defined herein) the first target antigen of interest, but not to the second molecule.

The terms ‘competing (with)’, ‘cross-blocking’, ‘cross-binding’ and ‘cross-inhibiting’ as used interchangeably herein, generally refer to a VHH, or a fragment thereof, according to the invention that can interfere with the binding of another VHH, or a fragment thereof, according to the invention or other molecule to human and/or murine FAP, as measured using a suitable in vitro or in vivo assay. A preferred cell used for testing the in vitro binding to human and/or murine FAP is a cell expressing human and/or murine FAP. A preferred cell line may be GM05389, U-87 MG or transfected HEK293 as defined earlier herein. Thus, more particularly, ‘competing (with)’, ‘cross-blocking’, ‘cross-binding’ and ‘cross-inhibiting’ using a VHH, or a fragment thereof, according to the invention may mean interfering with or competing with the binding of another VHH, or a fragment thereof, according to the invention with human and/or murine FAP, thereby reducing that binding by at least 10% but preferably at least 20%, for example by at least 50%, at least 60%, at least 70%, at least 80%, at least 90%, at least 95% or more, as measured using a suitable in vitro, cellular or in vivo assay, compared to the binding of that other VHH, or fragment thereof, according to the invention with human and/or murine FAP but without using the ‘cross-blocking’ VHH, or fragment thereof, according to the invention. In an embodiment, a VHH, or a fragment thereof, according to the inventio does not compete with the ligand of FAP for binding to it. As a result, the VHH, or the fragment thereof, is also expected not to interfere with the natural function of this receptor. It means that in an embodiment, a VHH, or a fragment thereof, according to the invention does not compete with the natural ligand of human and/or murine FAP and therefore is not inhibited to bind to human and/or murine FAP-expressing cells in vitro or in an in vivo or ex vivo setting.

In an embodiment, a VHH, or a fragment thereof, according to the invention does not substantially alter a FAP activity, it means it preferably does not substantially inhibit a FAP activity. Within the context of the invention, ‘substantial’ may mean at least 40%, at least 50%, at least 60%, at least 70%, at least 80%, at least 90%, at least 95% or 100% of a FAP activity is still detectable compared to the same FAP activity when the VHH, or the fragment thereof, is not present. Within the context of the invention, a FAP activity may be an exopeptidase and/or an endopeptidase activity. This exopeptidase activity may be a FAP dipeptidyl peptidase activity. In an embodiment, VHH, or a fragment thereof, according to the invention does not substantially inhibit the FAP dipeptidyl peptidase activity. This endopeptidase activity may be a gelatinase and/or collagenase activity. In an embodiment, VHH, or a fragment thereof, according to the invention does not substantially inhibit the FAP gelatinase and/or collagenase activity of FAP. Therefore, in an embodiment, VHH, or a fragment thereof, according to the invention is not a modulator of human and/or murine FAP. In an embodiment, it is not an inhibitor and it is not an activator of human and/or murine FAP. Any of the FAP activities may be assessed as illustrated in Juillerat-Jeanneret L. et al, (2017), Expert Opinion on Therapeutic Targets (http://dx.doi.org/10.1080/14728222.2017.1370455). The FAP dipeptidyl peptidase activity may be assessed using techniques known to the skilled person. In short, the human FAP enzymatic activity may be measured using the fluorogenic substrate benzyloxycarbonyl-Gly-Pro-7-amido-4-methylcoumarin (Z-Gly-Pro-AMC; Bachem).

A VHH, or a fragment thereof, according to the invention is said to show ‘cross-reactivity’ for two different target proteins of interest if it is specific for (as defined herein) both of these different target proteins of interest. In an embodiment, the two different target proteins of interest may be human and murine FAP.

Below we describe several preferred structural features (i.e. first, second, third, fourth structural features) of a VHH, or a fragment thereof, according to the invention. The VHH, or the fragment thereof, may be characterized by the presence of at least one, or all of these four structural features:
third and fourth structural features,
first and second structural features,
first, second and third structural features
third and fourth structural features,
third structural feature,
fourth structural feature,
first, third and fourth structural features,
Second, third and fourth structural features,
first and third structural features,
first and fourth structural features,
second and third structural features,
second and fourth structural features
first, second, third and fourth structural features.

First structural feature of VHH, or fragment thereof, according to the invention: based on the contacted region of FAP.

A first structural feature is that VHH, or fragment thereof, according to the invention contacts or binds or specifically binds or interacts to a region of human FAP comprised within amino acid 26 to 760 of SEQ ID NO: 5. The region within amino acid 26 to 760 of SEQ ID NO: 5 specifically bound or targeted by the VHH, or the fragment thereof, may be a linear region (i.e. linear epitope or sequential epitope) within said primary amino acid sequence. Alternatively said region may not be linear and may correspond to a conformational epitope. Usually a linear epitope comprises a linear sequence of amino acids that has a length of 5 to 30 amino acids, that is to say that it may have a length of 5, 6, 7, 8, 9, 10, 11, 12, 13, 14, 15, 16, 17, 18, 19, 20, 21, 22, 23, 24, 25, 26, 27, 28, 29 or 30 amino acids. Usually a conformational epitope is characterized by a number of non-consecutive amino acids within amino acid 26 to 760 of SEQ ID NO: 5 that come together in the three-dimensional tertiary structure of the protein and that are contacted by the VHH, or the fragment thereof.

In the following paragraph dedicated to the second structural feature of the VHH, or the fragment thereof, linear epitopes and conformational epitope of the VHH, or the fragment thereof, are defined.

Second structural feature of a VHH, or a fragment thereof, according to the invention: based on the epitope of the VHH, or the fragment thereof.

A VHH, or a fragment thereof, according to the invention that specifically binds an epitope of human and/or murine FAP may alternatively or in combination with the first structural feature defined above also be further defined by a second structural feature defined below.

A second structural feature is that a VHH, or a fragment thereof, according to the invention contacts or binds or specifically binds to a number of amino acids within amino acid 26 to 760 of SEQ ID NO: 5. These specific amino acids within amino acid 26 to 760 of SEQ ID NO: 5 are further defined below.

In a first embodiment of this second structural feature, a VHH, or a fragment thereof, according to the invention contacts or binds or specifically binds to at least one of amino acid comprised within 65-90 and/or 101-140 of SEQ ID NO: 5. Each combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65 or 66 amino acids from the 66 amino acids identified is therefore encompassed to be contacted by the VHH, or the fragment thereof.

In a second embodiment of this second structural feature, a VHH, or a fragment thereof, according to the invention contacts or binds or specifically binds to at least one of amino acid I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458 of SEQ ID NO: 5. Each combination of 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36 or 37 amino acids from the 37 amino acids identified is therefore encompassed to be contacted by the VHH, or the fragment thereof.

In a third embodiment of this second structural feature, the stretch of amino acids 65-90 of SEQ ID NO: 1 defines a first region of hFAP, which is contacted, bound or specifically bound by the VHH, or the fragment thereof. Not each amino acid within this stretch or region may be contacted, bound or specifically bound by the VHH, or the fragment thereof. In an embodiment, 1, 2, 3, 4, 5, 6, 7, 8, 9, 10, 11, 12, 13, 14, 15, 16, 17, 18, 19 or 20 amino acids of this stretch or region is contacted, bound or specifically bound by the VHH, or the fragment thereof. In an embodiment, this first stretch or region is an epitope of the VHH, or the fragment thereof. In an embodiment, an epitope of the VHH, or the fragment thereof is comprised within this first stretch or region.

Preferably within this first stretch at least one of Q65, E66, 176, V77, L78, Y79, N80, 181, E82, T83, G84, Q85, S86, Y87, T88, 189, L90 is contacted, bound or specifically bound by the VHH, or the fragment thereof. More preferably within this first stretch at least two of Q65, E66, 176, V77, L78, Y79, N80, 181, E82, T83, G84, Q85, S86, Y87, T88, 189, L90 is contacted, bound or specifically bound by the VHH, or the fragment thereof.

Even more preferably within this first stretch at least three of Q65, E66, 176, V77, L78, Y79, N80, 181, E82, T83, G84, Q85, S86, Y87, T88, 189 and L90 are contacted, bound or specifically bound by the VHH, or the fragment thereof.

Even more preferably, preferably, within this first stretch all Q65, E66, 176, V77, L78, Y79, N80, 181, E82, T83, G84, Q85, S86, Y87, T88, 189, L90 are contacted, bound or specifically bound by the VHH, or the fragment thereof.

Most preferably, within this first stretch all Q65, E66, 176, V77, L78, N80, 181, E82, T83, Q85, S86, Y87, T88, 189, L90 are contacted, bound or specifically bound by the VHH, or the fragment thereof.

In a fourth embodiment of this second structural feature, the stretch of amino acids 101-140 of SEQ ID NO: 5 defines a second region of hFAP, which is contacted, bound or specifically bound by the VHH, or the fragment thereof. Not each amino acid within this stretch or region needs to be contacted, bound or specifically bound by the VHH, or the fragment thereof. In an embodiment, 1, 2, 3, 4, 5, 6, 7, 8, 9, 10, 11, 12, 13, 14, 15, 16, 17, 18, 19, 20, 21, 22, 23, 24, 25, 26, 27, 28, 29 or 30 amino acids of this stretch or region is contacted, bound or specifically bound by the VHH, or the fragment thereof. In an embodiment, this second stretch or region is an epitope of the VHH, or the fragment thereof. In an embodiment, an epitope of the VHH, or the fragment thereof is comprised within this second stretch or region.

Preferably within this second stretch at least one of L105, S106, P107, D108, R109, Q110, F111, D134, L135, S136, N137 is contacted, bound or specifically bound by the VHH, or the fragment thereof.

More preferably within this second stretch at least two of L105, S106, P107, D108, R109, Q110, F111, D134, L135, S136, N137 is contacted, bound or specifically bound by the VHH, or the fragment thereof.

Even more preferably within this second stretch at least three of L105, S106, P107, D108, R109, Q110, F111, D134, L135, S136, N137 is contacted, bound or specifically bound by the VHH, or the fragment thereof.

Most preferably within this second stretch all L105, S106, P107, D108, R109, Q110, F111, D134, L135, S136, N137 are contacted, bound or specifically bound by the VHH, or the fragment thereof.

In a fifth embodiment of this second structural feature, the VHH, or the fragment thereof further contacts additional amino acids as I62, S63, G64, S91, V158, G159, R175, D457 and/or Y458 of SEQ ID NO: 5.

In a sixth embodiment of this second structural feature, each of the first and second stretches or regions defined above is contacted, bound or specifically bound by the VHH, or the fragment thereof. In an embodiment, the combination of these two stretches defines the conformational epitope of the VHH, or the fragment thereof. In an embodiment, a conformational epitope is comprised within the combination of these two stretches. Not each amino acid within each of these stretches or regions may be contacted, bound or specifically bound by the VHH, or the fragment thereof. In an embodiment, 1, 2, 3, 4, 5 or 6 amino acids (or more depending on the length of each stretch) of each of the stretches or regions is contacted, bound or specifically bound by the VHH, or the fragment thereof. In an embodiment, the VHH, or the fragment thereof further contacts additional amino acids as I62, S63, G64, S91, V158, G159, R175, D457 and/or Y458 of SEQ ID NO: 5.

In an embodiment, there is provided a VHH, or a fragment thereof, according to the invention that specifically binds an epitope of human FAP wherein the epitope is comprised within the amino acid stretch or region 65-90 and/or 101-140 of SEQ ID NO: 5. Optionally, the VHH, or the fragment thereof, further contacts additional amino acids as I62, S63, G64, S91, V158, G159, R175, D457 and/or Y458 of SEQ ID NO: 5.

In an embodiment, there is provided a VHH, or a fragment thereof, according to the invention that specifically binds an epitope of human FAP wherein the epitope is comprised within the combination of amino acid stretches or regions 65-90 and 101-140 of SEQ ID NO: 5. Optionally, the VHH, or the fragment thereof, further contacts additional amino acids as I62, S63, G64, S91, V158, G159, R175, D457 and/or Y458 of SEQ ID NO: 5.

In an embodiment, a VHH, or a fragment thereof, according to the invention contacts or binds or specifically binds to the following amino acids of SEQ ID NO: 5:
1) at least one, or at least two or at least three amino acids selected from: I62, S63, G64, Q65, E66, and/or
2) at least one, or at least two or at least three amino acids selected from: I76, V77, L78, Y79, N80, I81, E82, T83, G84, Q85, S86, Y87, T88, I89, L90, S91, and/or
3) at least one, or at least two or at least three amino acids selected from L105, S106, P107, D108, R109, Q110, F111, and/or
4) at least one, or at least two or at least three amino acids selected from D134, L135, S136, N137, and/or
5) V158 and/or G159 and/or
6) R175, and/or
7) D457 and/or Y458.

In an embodiment, a VHH, or a fragment thereof, according to the invention contacts or binds or specifically binds to the following amino acids of SEQ ID NO: 5:
1) at least one, or at least two or at least three amino acids selected from: I62, S63, G64, Q65, E66, and
2) at least one, or at least two or at least three amino acids selected from: I76, V77, L78, Y79, N80, I81, E82, T83, G84, Q85, S86, Y87, T88, I89, L90, S91, and
3) at least one, or at least two or at least three amino acids selected from L105, S106, P107, D108, R109, Q110, F111, and
4) at least one, or at least two or at least three amino acids selected from D134, L135, S136, N137, and
5) V158 and/or G159 and
6) R175, and
7) D457 and/or Y458.

In an embodiment, a VHH, or a fragment thereof, according to the invention contacts or binds or specifically binds to the following amino acids of SEQ ID NO: 5:
1) at least two or at least three amino acids selected from: I62, S63, G64, Q65, E66, and/or
2) at least two or at least three amino acids selected from: I76, V77, L78, Y79, N80, I81, E82, T83, G84, Q85, S86, Y87, T88, I89, L90, S91, and/or
3) at least two or at least three amino acids selected from L105, S106, P107, D108, R109, Q110, F111, and/or
4) at least two or at least three amino acids selected from D134, L135, S136, N137, and/or 5) V158 and G159 and/or
6) R175, and/or
7) D457 and Y458.

In an embodiment, a VHH, or a fragment thereof, according to the invention contacts or binds or specifically binds to the following amino acids of SEQ ID NO: 5:
1) at least two or at least three amino acids selected from: I62, S63, G64, Q65, E66, and
2) at least two or at least three amino acids selected from: I76, V77, L78, Y79, N80, I81, E82, T83, G84, Q85, S86, Y87, T88, I89, L90, S91, and
3) at least two or at least three amino acids selected from L105, S106, P107, D108, R109, Q110, F111, and
4) at least two or at least three amino acids selected from D134, L135, S136, N137, and
5) V158 and G159 and
6) R175, and
7) D457 and Y458.

In an embodiment, a VHH, or a fragment thereof, according to the invention contacts or binds or specifically binds to the following amino acids of SEQ ID NO: 5:
1) at least three amino acids selected from: I62, S63, G64, Q65, E66, and/or
2) at least three amino acids selected from: I76, V77, L78, Y79, N80, I81, E82, T83, G84, Q85, S86, Y87, T88, 189, L90, S91, and/or
3) at least three amino acids selected from L105, S106, P107, D108, R109, Q110, F111, and/or
4) at least three amino acids selected from D134, L135, S136, N137, and/or
5) V158 and G159 and/or
6) R175, and/or
7) D457 and Y458.

In an embodiment, a VHH, or a fragment thereof, according to the invention contacts or binds or specifically binds to the following amino acids of SEQ ID NO: 5:
1) at least three amino acids selected from: I62, S63, G64, Q65, E66, and
2) at least three amino acids selected from: I76, V77, L78, Y79, N80, I81, E82, T83, G84, Q85, S86, Y87, T88, I89, L90, S91, and
3) at least three amino acids selected from L105, S106, P107, D108, R109, Q110, F111, and
4) at least three amino acids selected from D134, L135, S136, N137, and
5) V158 and G159 and
6) R175, and
7) D457 and Y458.

In an embodiment, the following amino acids of SEQ ID NO: 5 are bound or contacted or interacted with a VHH, or a fragment thereof, according to the invention: I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and Y458.

In an embodiment, the following amino acids of SEQ ID NO: 5 are bound or contacted or interacted with a VHH, or a fragment thereof, according to the invention: I62, S63, G64, Q65, E66, I76, V77, L78, N80, I81, E82, T83, Q85, S86, Y87, T88, I89, L90, S91, L105, S106, P107, D108, R109, Q110, F111, D134, L135, S136, N137, V158, G159, R175, D457 and Y458.

Third Structural Feature of a VHH, or a Fragment Thereof, According to the Invention: Based on the Full Length Sequence

A third structural feature is that a VHH, or a fragment thereof, according to the invention relates to the full length amino acid sequence. The present invention discloses a family of structurally closely related VHHs represented by an amino acid sequence comprising, consisting of or essentially consisting of SEQ ID NO: 4 or a fragment thereof. VHH VHH1 is represented by SEQ ID NO: 4 (see table below).

In an embodiment, a VHH, or a fragment thereof, according to the invention may be defined by its first structural feature as defined above and its third structural feature further defined below.

In an embodiment, a VHH, or a fragment thereof, according to the invention may be defined by its second structural feature as defined above and its third structural feature further defined below.

In a first embodiment of this third structural feature, a VHH, or a fragment thereof, according to the invention is represented by an amino acid sequence that comprises or essentially consists of an amino acid sequence having at least 80% sequence identity with SEQ ID NO: 4 or a portion thereof. In an embodiment, the sequence identity with this sequence is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment of this third structural feature, a VHH, or a fragment thereof, according to the invention is represented by an amino acid sequence that comprises or essentially consists of an amino acid sequence having at least 81% sequence similarity with SEQ ID NO: 4 or a portion thereof. In an embodiment, the sequence similarity with this sequence is at least 81%, 82%, 83%, 84%, 85%, 86%, 87%, 88%, 89%, 90%, 91%, 92%, 93%, 94%, 95%, 96%, 97%, 98%, 99% or 100%.

In a second embodiment of this third structural feature, a VHH, or a fragment thereof, according to the invention is represented by an amino acid sequence that comprises or essentially consists of an amino acid sequence having at least 80% sequence identity with SEQ ID NO:4 or a portion thereof and has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

In an embodiment of this third structural feature, a VHH, or a fragment thereof, according to the invention is represented by an amino acid sequence that comprises or essentially consists of an amino acid sequence having at least 81% sequence similarity with SEQ ID NO:4 or a portion thereof and has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

For example a tag such as a His-tag may be added to a VHH, or a fragment thereof, according to the invention. Usually His-tag comprises 4, 5, 6, 7, 8, 9, 10 histidines. Alternative tag may be a Hemagglutinin tag (HA-tag): YPYDVPDYA (SEQ ID NO: 11); YPYDVPDYGS (SEQ ID NO: 12) or a cysteine tag (Cys tag). A cysteine tag is a tag that comprises one or several cysteines. Non-limiting examples of cysteine tags are C; GGC; SPSTPPTPSPSTPPC (SEQ ID NO: 13)

The way identity and similarity are assessed is explained in detail in the part dedicated to definition at the end of the description. Usually when identity is defined by reference to a SEQ ID NO, said identity is assessed over the whole SEQ ID NO. However, it is also encompassed by the invention that identity (or similarity) is assessed over a portion (or a fragment) of said sequence. Within this context, a portion may mean at least 50%, 60%, 70%, 80%, 90%, 95% of the length of the SEQ ID NO. The length of the sequence encompassed may still be longer than the length of the SEQ ID NO used to assess the identity (or similarity) (i.e. length being at least 50% of the length of the SEQ ID NO, 60%, 70%, 80%, 90%, the same as the one of the SEQ ID NO even though the identity (or similarity) is assessed over a portion of this SEQ ID NO, or the length being 1, 5, 10, 15, 20, 25, 30, 35, 40, 45, 50, 55, 60 amino acids longer than the exact length of SEQ ID NO.

In a third embodiment of this third structural feature, the length of the VHH, or the fragment thereof, is from 110 to 130 amino acids or 110, 111, 112, 113, 114, 115, 116, 117, 118, 119, 120, 121, 122, 123, 124, 125, 126, 127, 128, 129 or 130 amino acids. This length does not include the length of a tag, such as a His tag that may be added to the sequence of the VHH, or the fragment thereof.

In an embodiment, a VHH, or a fragment thereof, according to the invention has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO:4.

In an embodiment, a VHH, or a fragment thereof, according to the invention has a length which is ranged from 110 to 130 amino acids or 110, 111, 112, 113, 114, 115, 116, 117, 118, 119, 120, 121, 122, 123, 124, 125, 126, 127, 128, 129 or 130 amino acids and comprises SEQ ID NO: 1, SEQ ID NO: 2 and SEQ ID NO: 3.

In a fourth embodiment of this third structural feature, a VHH, or a fragment thereof, according to the invention is represented by an amino acid sequence that comprises or essentially consists of an amino acid sequence having at least 80% sequence identity with at least one of SEQ ID NO: 4 or a portion thereof and the length of the VHH, or the fragment thereof, is from 80 to 150 amino acids or 90 to 140 or 100 to 130 or 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity with at least of one of these sequences is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment of this third structural feature, a VHH, or a fragment thereof, according to the invention is represented by an amino acid sequence that comprises or essentially consists of an amino acid sequence having at least 81% sequence similarity with at least one of SEQ ID NO: 4 or a portion thereof and the length of the VHH, or the fragment thereof, is from 80 to 150 amino acids or 90 to 140 or 100 to 130 or 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence similarity with at least of one of these sequences is at least 81%, 82%, 83%, 84%, 85%, 86%, 87%, 88%, 89%, 90%, 91%, 92%, 93%, 94%, 95%, 96%, 97%, 98%, 99% or 100%.

In an embodiment, a VHH, or a fragment thereof, according to the invention contacts, binds or specifically binds at least one of (preferably both) the stretches or regions of amino acids of SEQ ID NO: 5 as defined earlier herein (i.e. amino acid stretch or region 65-90 and/or 101-140 of SEQ ID NO: 5). In an embodiment, an epitope of said VHH, or fragment thereof, is comprised within these stretches or regions of amino acids of SEQ ID NO: 5.

Moreover, in an embodiment, a VHH, or a fragment thereof, according to the invention has for conformational epitope the combination of stretches or regions of amino acids of SEQ ID NO: 5 as defined earlier herein (i.e. amino acid stretches or regions 65-90 and/or 101-140 of SEQ ID NO: 5). In an embodiment, a conformational epitope of said VHH, or fragment thereof, is comprised within these stretches or regions of amino acids of SEQ ID NO: 5.

These epitopes define a family of VHHs or fragments thereof. This family of VHHs or fragments thereof shares at least one of these epitopes, linear epitopes and/or this conformational epitope.

In an embodiment, a VHH, or a fragment thereof, according to the invention:
is represented by an amino acid sequence that comprises or essentially consists of an amino acid sequence having at least 80% sequence identity (or similarity) with SEQ ID NO: 4 or a portion thereof (third structural feature) and
which has for epitope the amino acid stretch or region comprised within 65-90 and/or 101-140 of SEQ ID NO: 5 (second structural feature).

In an embodiment, the sequence identity (or similarity) with this sequence is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a VHH, or a fragment thereof, according to the invention has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO: 4.

In an embodiment, a VHH, or a fragment thereof, according to the invention:
is represented by an amino acid sequence that comprises or essentially consists of an amino acid sequence having at least 80% sequence identity (or similarity) with SEQ ID NO: 4 or a portion thereof (third structural feature) and
contacts or binds or specifically binds to at least one of amino acid I62, S63, G64, Q65, E66, 176, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO: 5. (second structural feature).

In an embodiment, the sequence identity (or similarity) with this sequence is at least 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a VHH, or a fragment thereof, according to the invention has a length which is ranged from the exact length of SEQ ID NO: 4 or 1, 2, 3, 4, 5, 6, 7, 8, 9, 10, 15, 20, 25, 30, 35, 40, 45, 50, 55, 60 amino acids longer than the exact length of SEQ ID NO: 4.

Each of the other embodiments of the second structural feature may be combined with each embodiment of the third structural feature.

### Fourth Structural Feature: CDR/CDR Grafting

In a fourth structural feature, a VHH, or a fragment thereof, according to the invention is represented by an amino acid sequence that comprises at least one combination of CDR sequences chosen from the group comprising:
a CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. One or two amino acids of said CDR1, CDR2 and/or CDR3 may have been substituted by another one amino acid without substantially altering the activity of the obtained VHH, or fragment thereof. The same holds for any of the frame work regions of the VHH, or fragment thereof. Each of these variants are also encompassed within the invention. An activity of said variant is a specific binding activity as earlier defined herein. Within the context of the invention, ‘substantial’ may mean at least 40%, at least 50%, at least 60%, at least 70%, at least 80%, at least 90%, at least 95% or 100% of said binding activity is still detectable compared to the activity of the VHH, or the fragment thereof, with the initial CDR and/or FR regions.

Thus, in particular embodiments, the present invention provides a VHH, or a fragment thereof, according to the invention comprising the heavy chain antibodies with the (general) structure or which is derived therefrom:

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and are as further defined herein (see table 2 listing the amino acid sequences of heavy chain variable domains that have been raised against human and/or murine FAP).

TABLE 2
CDR and FR of VHH VHH1
(using the IMGT nomenclature as defined later
on herein in the general part of the description
dedicated to the general definition
of the invention)

| | Amino acid sequence |
|---|---|
| VHH1 (SEQ ID NO: 4) | DVQLVESGGGLVQPGGSLRLSCVASGR |
| | LSSSNSMAWYRQVPGKRRELVAGITGG |
| | GETNYADFVGGRFTISRDNAKNGLYLQ |
| | LNGLKPEDTAAYYCNFWPPLINYWGQG |
| | TQVTVSS |
| CDR of VHH1 | |
| CDR1 (SEQ ID NO: 1) | GRLSSSNS |
| CDR2 (SEQ ID NO: 2) | ITGGGET |
| CDR3 (SEQ ID NO: 3) | NFWPPLINY |
| FR of VHH1 | |
| FR1 (SEQ ID NO: 7) | DVQLVESGGGLVQPGGSLRLSCVAS |
| FR2 (SEQ ID NO: 8) | MAWYRQVPGKRRELVAG |
| FR3 (SEQ ID NO: 9) | NYADFVGGRFTISRDNAKNGLYLQLNG |
| | LKPEDTAAYYC |
| FR4 (SEQ ID NO: 10) | WGQGTQVTVSS |

It should be noted that the invention is not limited as to the origin of a VHH, or a fragment thereof, according to the invention (or of the nucleotide sequences to express these), nor as to the way that the the VHH, or the fragment thereof, or nucleotide sequences disclosed herein are (or have been) generated or obtained. Thus, a VHH, or a fragment thereof, according to the invention may be naturally occurring amino acid sequences (from any suitable species) or synthetic or semi-synthetic amino acid sequences. Methods for isolating a VHH, or a fragment thereof, and methods of producing a VHH, or a fragment thereof, as well as nucleic acid molecule encoding the VHH, or the fragment thereof, constructs comprising these nucleic acid molecules and cells comprising these constructs are disclosed in detail in the definition part at the end of the description.

In a specific but non-limiting aspect of the invention, the amino acid sequence of the VHH, or the fragment thereof, is a naturally occurring immunoglobulin sequence (from any suitable species) or a synthetic or semi-synthetic immunoglobulin sequence, including but not limited to “humanized” immunoglobulin sequences (such as partially or fully humanized mouse or rabbit immunoglobulin sequences, and in particular partially or fully humanized VHH sequences), “camelized” immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Also, a VHH, or a fragment thereof, according to the invention may be suitably humanized, as further described herein, so as to provide one or more further (partially or fully) humanized amino acid sequences of the invention.

In an embodiment, a VHH, or a fragment thereof, according to the invention is derived from the VHH, or the fragment thereof, described above using CDR grafting.

A preferred VHH, or a fragment thereof, according to the invention comprises a:
CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. FR regions may be as identified in table 2. Alternatively, FR regions may be from another VHH, or fragment thereof.
CDR1 region comprising or consisting of or essentially consisting of SEQ ID NO: 1 and a CDR3 region comprising or consisting of or essentially consisting of SEQ ID NO: 3, FR regions may be as identified in table 2. Alternatively, FR regions may be from another VHH, or fragment thereof. CDR2 region is from another VHH, or fragment thereof.
CDR2 region comprising or consisting of or essentially consisting of SEQ ID NO: 2 and a CDR3 region comprising or consisting of or essentially consisting of SEQ ID NO: 3, FR regions may be as identified in table 2. Alternatively, FR regions may be from another VHH, or fragment thereof. CDR1 region is from another VHH, or fragment thereof.
CDR1 region comprising or consisting of or essentially consisting of SEQ ID NO: 1 and a CDR2 region comprising or consisting of or essentially consisting of SEQ ID NO: 2, FR regions may be as identified in table 2. Alternatively, FR regions may be from another VHH, or fragment thereof. CDR3 region is from another VHH, or fragment thereof.
CDR1 region comprising or consisting of or essentially consisting of SEQ ID NO: 1 and it comprises a CDR2 and CDR3 regions from another VHH, or fragment thereof, and may have the FR of as identified in table 2. Alternatively, FR regions may be from another VHH, or fragment thereof.
CDR2 region comprising or consisting of or essentially consisting of SEQ ID NO: 2 and it comprises a CDR1 and CDR3 regions from another VHH, or fragment thereof, and may have the FR as identified in table 2. Alternatively, FR regions may be from another VHH, or fragment thereof.
CDR3 region comprising or consisting of or essentially consisting of SEQ ID NO: 3 and it comprises a CDR1 and CDR2 regions from another VHH, or fragment thereof, and may have the FR as identified in table 2. Alternatively, FR regions may be from another VHH, or fragment thereof.

Similarly, when an amino acid sequence comprises a synthetic or semi-synthetic sequence (such as a partially humanized sequence), said sequence may optionally be further suitably humanized, again as described herein, so as to provide one or more further (partially or fully) humanized amino acid sequences as disclosed herein. At the end of the description, a more detailed definition of “agonist” “antagonist”, “variants”, “posttranslational structural characterization” is provided.

In particular, humanized VHH, or a fragment thereof, according to the invention may be represented by amino acid sequences in which at least one amino acid residue is present (and in particular, in at least one of the framework residues) that is and/or that corresponds to a humanizing substitution. In addition, or alternatively, other potentially useful humanizing substitutions can be ascertained by comparing the sequence of the framework regions of a naturally occurring VHH sequence with the corresponding framework sequence of one or more closely related human V_{H} sequences, after which one or more of the potentially useful humanizing substitutions (or combinations thereof) thus determined can be introduced into said VHH sequence (in any manner known per se, as further described herein) and the resulting humanized VHH sequences or functional fragments thereof can be tested for affinity for the target, for stability, for ease and level of expression, and/or for other desired properties. In this way, by means of a limited degree of trial and error, other suitable humanizing substitutions (or suitable combinations thereof) can be determined by the skilled person.

In an embodiment, a VHH, or a fragment thereof, according to the invention may be defined by its first structural feature as defined above and its fourth structural feature further defined herein.

In an embodiment, a VHH, or a fragment thereof, according to the invention may be defined by its second structural feature as defined above and its fourth structural feature further defined herein.

In an embodiment, a VHH, or a fragment thereof, according to the invention may be defined by its second structural feature as defined above, its third structural feature and its fourth structural feature further defined herein.

In an embodiment, a VHH, or a fragment thereof, according to the invention contacts, binds or specifically binds at least one of (preferably both) the stretches or regions of amino acids of SEQ ID NO: 5 as defined earlier herein (i.e. amino acid stretch or region 65-90 and/or 101-140 of SEQ ID NO: 5). In an embodiment, an epitope of said VHH, or fragment thereof, is comprised within these stretches or regions of amino acids of SEQ ID NO: 5.

Moreover, a VHH, or a fragment thereof, according to the invention may have for conformational epitope the combination of stretches or regions of amino acids of SEQ ID NO: 5 as defined earlier herein (i.e. amino acid stretches or regions 65-90 and/or 101-140 of SEQ ID NO: 5). In an embodiment, a conformational epitope of said VHH, or fragment thereof, is comprised within the combination of these stretches or regions of amino acids of SEQ ID NO: 5.

These epitopes define a family of VHHs, or fragments thereof. This family shares at least one of these epitopes, linear epitopes and/or this conformational epitope.

Preferred a VHH, or a fragment thereof, according to the invention comprises a:
CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. FR regions may be as identified in table 2. Alternatively, FR regions may be from another VHH, or fragment thereof.
CDR1 region comprising or consisting of or essentially consisting of SEQ ID NO: 1 and a CDR3 region comprising or consisting of or essentially consisting of SEQ ID NO: 3, FR regions may be as identified in table 2. Alternatively, FR regions may be from another VHH, or fragment thereof. CDR2 region is from another VHH, or fragment thereof.
CDR2 region comprising or consisting of or essentially consisting of SEQ ID NO: 2 and a CDR3 region comprising or consisting of or essentially consisting of SEQ ID NO: 3, FR regions may be as identified in table 2. Alternatively, FR regions may be from another VHH, or fragment thereof. CDR1 region is from another VHH, or fragment thereof.
CDR1 region comprising or consisting of or essentially consisting of SEQ ID NO: 1 and a CDR2 region comprising or consisting of or essentially consisting of SEQ ID NO: 2, FR regions may be as identified in table 2. Alternatively, FR regions may be from another VHH, or fragment thereof. CDR3 region is from another VHH, or fragment thereof.
CDR1 region comprising or consisting of or essentially consisting of SEQ ID NO: 1 and it comprises a CDR2 and CDR3 regions from another VHH, or fragment thereof and may have the FR of as identified in table 2. Alternatively, FR regions may be from another VHH, or fragment thereof.
CDR2 region comprising or consisting of or essentially consisting of SEQ ID NO: 2 and it comprises a CDR1 and CDR3 regions from another VHH, or fragment thereof and may have the FR as identified in table 2. Alternatively, FR regions may be from another VHH, or fragment thereof.
CDR3 region comprising or consisting of or essentially consisting of SEQ ID NO: 3 and it comprises a CDR1 and CDR2 regions from another VHH, or fragment thereof and may have the FR as identified in table 2. Alternatively, FR regions may be from another VHH, or fragment thereof (fourth structural feature)
   and
has an epitope comprised within the amino acid stretch or region comprised within 65-90 and/or 101-140 of SEQ ID NO: 5 (second structural feature).

A preferred VHH, or a fragment thereof, according to the invention comprises a:
CDR1 region comprising or consisting of or essentially consisting of SEQ ID NO: 1, a CDR2 region comprising or consisting of or essentially consisting of SEQ ID NO: 2, and a CDR3 region comprising or consisting of or essentially consisting of SEQ ID NO: 3. FR regions may be as identified in table 2. Alternatively, FR regions may be from another VHH, or fragment thereof.
CDR1 region comprising or consisting of or essentially consisting of SEQ ID NO: 1 and a CDR3 region comprising or consisting of or essentially consisting of SEQ ID NO: 3, FR regions may be as identified in table 2. Alternatively, FR regions may be from another VHH, or fragment thereof. CDR2 region is from another VHH, or fragment thereof.
CDR2 region comprising or consisting of or essentially consisting of SEQ ID NO: 2 and a CDR3 region comprising or consisting of or essentially consisting of SEQ ID NO: 3, FR regions may be as identified in table 2. Alternatively, FR regions may be from another VHH, or fragment thereof. CDR1 region is from another VHH, or fragment thereof.
CDR1 region comprising or consisting of or essentially consisting of SEQ ID NO: 1 and a CDR2 region comprising or consisting of or essentially consisting of SEQ ID NO: 2, FR regions may be as identified in table 2. Alternatively, FR regions may be from another VHH, or fragment thereof. CDR3 region is from another VHH, or fragment thereof.
CDR1 region comprising or consisting of or essentially consisting of SEQ ID NO: 1 and it comprises a CDR2 and CDR3 regions from another VHH, or fragment thereof and may have the FR of as identified in table 2. Alternatively, FR regions may be from another VHH, or fragment thereof.
CDR2 region comprising or consisting of or essentially consisting of SEQ ID NO: 2 and it comprises a CDR1 and CDR3 regions from another VHH, or fragment thereof, and may have the FR as identified in table 2. Alternatively, FR regions may be from another VHH, or fragment thereof.
CDR3 region comprising or consisting of or essentially consisting of SEQ ID NO: 3 and it comprises a CDR1 and CDR2 regions from another VHH, or fragment thereof and may have the FR as identified in table 2. Alternatively, FR regions may be from another VHH, or fragment thereof (fourth structural feature)
   and
contacts or binds or specifically binds to at least one of amino acid I62, S63, G64, Q65, E66, I76, V77, L78, Y79, N80, I81, E82, T83, G84, Q85, S86, Y87, T88, 189, L90, S91, L105, S106, P107, D108, R109, Q110, F111, D134, L135, S136, N137, V158, G159, R175, D457 and/or Y458 of SEQ ID NO: 5. (second structural feature).

Each of the other embodiments of the second structural feature may be combined with each embodiment of the fourth structural feature.

In an embodiment, a VHH, or a fragment thereof, according to the invention is represented by a first and/or second and/or third and/or fourth structural feature as identified herein. Alternatively or in combination with said structural feature, said VHH, or fragment thereof, is characterized by at least one of the following functional features:
which is to specifically bind human and/or murine FAP, preferably which is to specifically bind human and murine FAP and
which is not to modulate a FAP activity.

The specific binding has been described earlier herein. Most preferably, a VHH, or a fragment thereof, according to the invention specifically binds to human and/or murine FAP with a K_{D} ranged from 10⁻⁹ to 10⁻¹² moles/liter and/or a k_{off} ranging from 10⁻² to 10⁻⁵ s⁻¹ preferably assessed using bio-layer interferometry, more preferably with a K_{D} ranged from 10⁻⁹ to 10-12 moles/liter and a K_{off} ranging from 10⁻² to 10⁻⁵ s⁻¹.

The second functional feature relating to the fact the VHH, or the fragment thereof, may not be a modulator (i.e. is not an inhibitor, is not an activator of human and/or murine FAP) has also been described in detail herein.

In an embodiment, a VHH, or a fragment thereof, according to the invention should therefore fulfil at least one of the structural features and/or at least one of the functional features.

In embodiments, a combination according to the invention comprises:
a VHH, or a fragment thereof, according to the invention which specifically binds human and/or murine FAP, wherein the epitope is comprised within the amino acid stretch or region 65-90 and/or 101-140 of SEQ ID NO: 5; and a labelled compound comprising iodine-131 and GMIB,
a VHH, or a fragment thereof, according to the invention which specifically binds human and/or murine FAP, wherein the epitope is comprised within the amino acid stretch or region 65-90 and/or 101-140 of SEQ ID NO: 5; and a labelled compound comprising DOTA or DTPA and lutetium-177, preferably wherein the labelled compound can be represented by ¹⁷⁷Lu-DOTA-PEG₇-Tz or ¹⁷⁷Lu-DTPA-PEG₇-Tz,
a VHH, or a fragment thereof, according to the invention which specifically binds human and/or murine FAP, wherein the epitope is comprised within the amino acid stretch or region 65-90 and/or 101-140 of SEQ ID NO: 5; and a labelled compound comprising DOTA and actinium-225, preferably wherein the labelled compound can be represented by ²²⁵Ac-DOTA-PEG₇-Tz, or
a VHH, or a fragment thereof, according to the invention which specifically binds human and/or murine FAP, wherein the epitope is comprised within the amino acid stretch or region 65-90 and/or 101-140 of SEQ ID NO: 5; and a labelled compound comprising technetium-99m.

In embodiments, a combination according to the invention comprises:
a VHH, or a fragment thereof, according to the invention which specifically binds human and/or murine FAP, wherein the conformational epitope is comprised within the combination of amino acid stretch or region 65-90 and 101-140 of SEQ ID NO: 5; and a labelled compound comprising GMIB and iodine-131,
a VHH, or a fragment thereof, according to the invention which specifically binds human and/or murine FAP, wherein the conformational epitope is comprised within the combination of amino acid stretch or region 65-90 and 101-140 of SEQ ID NO: 5; and a labelled compound comprising DOTA or DTPA and lutetium-177, preferably wherein the labelled compound can be represented by ¹⁷⁷Lu-DOTA-PEG₇-Tz or ¹⁷⁷Lu-DTPA-PEG₇-Tz,
a VHH, or a fragment thereof, according to the invention which specifically binds human and/or murine FAP, wherein the conformational epitope is comprised within the combination of amino acid stretch or region 65-90 and 101-140 of SEQ ID NO: 5; and a labelled compound comprising DOTA and actinium-225, preferably wherein the labelled compound can be represented by ²²⁵Ac-DOTA-PEG₇-Tz, or
a VHH, or a fragment thereof, according to the invention which specifically binds human and/or murine FAP, wherein the conformational epitope is comprised within the combination of amino acid stretch or region 65-90 and 101-140 of SEQ ID NO: 5; and a labelled compound comprising technetium-99m.

In embodiments, a combination according to the invention comprises:
a VHH, or a fragment thereof, according to the invention which specifically binds human and/or murine FAP, wherein at least one of amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO: 5 interacts with said VHH or fragment; and a labelled compound comprising GMIB and iodine-131,
a VHH, or a fragment thereof, according to the invention which specifically binds human and/or murine FAP, wherein at least one of amino acids I62, S63, G64, Q65, E66, I76, V77, L78, Y79, N80, I81, E82, T83, G84, Q85, S86, Y87, T88, 189, L90, S91, L105, S106, P107, D108, R109, Q110, F111, D134, L135, S136, N137, V158, G159, R175, D457 and/or Y458 of SEQ ID NO: 5 interacts with said VHH or fragment; and a labelled compound comprising DOTA or DTPA and lutetium-177, preferably wherein the labelled compound can be represented by ¹⁷⁷Lu-DOTA-PEG₇-Tz or ¹⁷⁷Lu-DTPA-PEG₇-Tz,
a VHH, or a fragment thereof, according to the invention which specifically binds human and/or murine FAP, at least one of amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO: 5 interacts with said VHH or fragment; and a labelled compound comprising DOTA and actinium-225, preferably wherein the labelled compound can be represented by ²²⁵Ac-DOTA-PEG₇-Tz, or
a VHH, or a fragment thereof, according to the invention which specifically binds human and/or murine FAP, at least one of amino acids I62, S63, G64, Q65, E66, I76, V77, L78, Y79, N80, I81, E82, T83, G84, Q85, S86, Y87, T88, I89, L90, S91, L105, S106, P107, D108, R109, Q110, F111, D134, L135, S136, N137, V158, G159, R175, D457 and/or Y458 of SEQ ID NO: 5 interacts with said VHH or fragment; and a labelled compound comprising technetium-99m.

In embodiments, a combination according to the invention comprises:
a VHH, or a fragment thereof, according to the invention that specifically binds human and/or murine FAP, wherein said VHH or fragment is represented by an amino acid sequence having at least 80% sequence identity (or similarity) with at least one of SEQ ID NO: 1, 2, 3, 4 or a portion thereof; and a labelled compound comprising GMIB and iodine-131,
a VHH, or a fragment thereof, according to the invention that specifically binds human and/or murine FAP, wherein said VHH or fragment is represented by an amino acid sequence having at least 80% sequence identity (or similarity) with at least one of SEQ ID NO: 1, 2, 3, 4 or a portion thereof; and a labelled compound comprising DOTA or DTPA and lutetium-177, preferably wherein the labelled compound can be represented by ¹⁷⁷Lu-DOTA-PEG₇-Tz or ¹⁷⁷Lu-DTPA-PEG₇-Tz, a VHH, or a fragment thereof, according to the invention that specifically binds human and/or murine FAP, wherein said VHH or fragment is represented by an amino acid sequence having at least 80% sequence identity (or similarity) with at least one of SEQ ID NO: 1, 2, 3, 4 or a portion thereof; and a labelled compound comprising DOTA and actinium-225, preferably wherein the labelled compound can be represented by ²²⁵Ac-DOTA-PEG₇-Tz, or
a VHH, or a fragment thereof, according to the invention that specifically binds human and/or murine FAP, wherein said VHH or fragment is represented by an amino acid sequence having at least 80% sequence identity (or similarity) with at least one of SEQ ID NO: 1, 2, 3, 4 or a portion thereof; and a labelled compound comprising technetium-99m.

### Human Folate Receptor Alpha (FOLR1)

In embodiments, a VHH, or a fragment thereof, according to the invention is particularly suited for binding to human folate receptor alpha (FOLR1).

Example 8 demonstrates that TCO-conjugated FOLR1-binding VHHs can be used in pre-targeting applications.

In an embodiment, a VHH, or a fragment thereof, according to the invention specifically binds human folate receptor alpha (FOLR1, which is represented by SEQ ID NO: 14), but does neither specifically bind human folate receptor beta (FOLR2) nor human folate receptor gamma (FOLR3) and is further characterized by an additional (structural) feature as defined herein (such as first, second, third and/or fourth structural features).

In an embodiment, a VHH, or a fragment thereof, according to the invention specifically binds human folate receptor alpha (FOLR1, which is represented by SEQ ID NO: 14), but does neither specifically bind murine FOLR1 nor human folate receptor beta (FOLR2) nor human folate receptor gamma (FOLR3) and is further characterized by an additional (structural) feature as defined herein (such as first, second, third and/or fourth structural features).

In an embodiment, a VHH, or a fragment thereof, according to the invention specifically binds human folate receptor alpha (FOLR1, which is represented by SEQ ID NO: 14), but does neither specifically bind murine FOLR1 nor human folate receptor beta (FOLR2) nor human folate receptor gamma (FOLR3) (or the VHH or fragment thereof specifically binds human FOLR1 but does neither specifically bind human FOLR2 nor human FOLR3) and fulfils at least one of the following:
d. the epitope is comprised within amino acid 25 to 233 of SEQ ID NO: 14,
e. at least amino acid C89, G90, E91, M92, A93, P94, E140, Q141, W142, W143, E144, D145, C146, R147, T148, S149, Y150, Q176, P177, F178, H179, F180, Y181, F182, P183 and/or T184 of SEQ ID NO: 14 interacts with the VHH or fragment thereof,
f. the VHH or fragment thereof is represented by an amino acid sequence that comprises an amino acid sequence having at least 60% sequence identity with at least one of SEQ ID NO: 15, 16, 17, 18, 19, 20, 21 or 22 or a portion thereof.

In an embodiment, there is provided a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or the VHH or fragment thereof specifically binds human FOLR1 but does neither specifically bind human FOLR2 nor human FOLR3) and the VHH or fragment thereof fulfils a) and/or b):
a. the epitope is comprised within amino acid 25 to 233 of SEQ ID NO: 14 and the VHH or fragment thereof specifically binds to the following amino acids of SEQ ID NO: 14:
   1) at least one, or at least two, or at least three amino acids selected from C89, G90, E91, M92, A93, or P94, and/or
   2) at least one, or at least two, or at least three amino acids selected from E140, Q141, W142, W143, E144, D145, C146, R147, T148, S149, or Y150, and/or
   3) at least one, or at least two, or at least three amino acids selected from Q176, P177, F178, H179, F180, Y181, F182, P183, or T184,
b. the VHH or fragment thereof is represented by an amino acid sequence that comprises an amino acid sequence having at least 63% sequence identity with at least one of SEQ ID NO: 21, 22, 16, 17, 18, 19, or 20 over the full length of said sequence or over at least 50% of the length of said sequence.

In an embodiment, there is provided a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3), (or the VHH or fragment thereof specifically binds human FOLR1 but does neither specifically bind human FOLR2 nor human FOLR3), wherein the epitope is comprised within amino acid 25 to 233 of SEQ ID NO: 14 and the VHH or fragment thereof specifically binds to the following amino acids of SEQ ID NO: 14:
1) at least one, or at least two, or at least three amino acids selected from C89, G90, E91, M92, A93, or P94, and
2) at least one, or at least two, or at least three amino acids selected from E140, Q141, W142, W143, E144, D145, C146, R147, T148, S149, or Y150, and
3) at least one, or at least two, or at least three amino acids selected from Q176, P177, F178, H179, F180, Y181, F182, P183, or T184.

In an embodiment, there is provided a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3), wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

In an embodiment, there is provided a VHH, or a fragment thereof, according to the invention that specifically binds an epitope of human folate receptor alpha (FOLR1), but does neither bind human folate receptor beta (FOLR2) nor human folate receptor gamma (FOLR3), wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

TABLE 3
Amino acid sequence of human FOLR1
(Uniprot entry P15328)

| | |
|---|---|
| SEQ ID NO: 14 | MAQRMTTQLLLLLVWVAVVGEAQTRIAWARTELLNV |
| | CMNAKHHKEKPGPEDKLHEQCRPWRKNACCSTNTSQ |
| | EAHKDVSYLYRFNWNHCGEMAPACKRHFIQDTCLYE |
| | CSPNLGPWIQQVDQSWRKERVLNVPLCKEDCEQWWE |
| | DCRTSYTCKSNWHKGWNWTSGFNKCAVGAACQPFHF |
| | YFPTPTVLCNEIWTHSYKVSNYSRGSGRCIQMWFDP |
| | AQGNPNEEVARFYAAAMSGAGPWAAWPFLLSLALML |
| | LWLLS |

Within the context of the invention, Folate receptor alpha also known as folate receptor 1 or folate binding protein is denominated using the abbreviation FOLR1 or FR-alpha at the protein and at the gene levels. Officially, FR-alpha is the recommended protein name from Uniprot and FOLR1 is used for the gene name.

The binding to human FOLR1 may be assessed in a homogeneous mixture of different antigens. In certain embodiments, a specific binding interaction will discriminate between desirable and undesirable antigens in a sample, in some embodiments more than about 10 to 100-fold or more (e.g., more than about 1000- or 10,000-fold).

In an embodiment, the binding may be assessed in vitro using cells expressing human FOLR1, and optionally in vivo or ex vivo as earlier defined herein. These cells may be human cells and expressing endogenous human FOLR1. Alternatively, these cells may overexpress human FOLR1. Cells overexpressing human FOLR1 may be human or non-human cells. Preferred cells are SKOV3 and OVCAR3.

In an embodiment, a VHH, or a fragment thereof, according to the invention specifically binds to human FOLR1 and does not specifically bind to murine FOLR1 or to human FOLR2 or to human FOLR3. This assessment is preferably carried out using ELISA or SPR.

It is also expected that the VHH or fragment thereof will bind to a number of naturally occurring or synthetic analogues, variants, mutants, alleles, parts and fragments of human FOLR1.

In an embodiment, a VHH, or a fragment thereof, according to the invention will specifically bind to at least those analogues, variants, mutants, alleles, naturally occurring, synthetic analogues, parts and fragments of human FOLR1 that (still) contain the epitope of the (natural/wild-type) antigen to which the VHH or fragment thereof binds. The epitope of human FOLR1 for the VHH or fragment thereof of the invention is comprised within amino acid 25 to 233 of SEQ ID NO: 14.

In an embodiment, at least one of the following amino acids of this part of SEQ ID NO: 14 is bound by the VHH or fragment thereof: C89, G90, E91, M92, A93, P94, E140, Q141, W142, W143, E144, D145, C146, R147, T148, S149, Y150, Q176, P177, F178, H179, F180, Y181, F182, P183 and/or T184 of SEQ ID NO: 14.

In another embodiment, a VHH, or a fragment thereof, according to the invention specifically binds to the following amino acids of SEQ ID NO: 14:
1) at least one, or at least two, or at least three amino acids selected from C89, G90, E91, M92, A93, or P94, and/or
2) at least one, or at least two, or at least three amino acids selected from E140, Q141, W142, W143, E144, D145, C146, R147, T148, S149, or Y150, and/or
3) at least one, or at least two, or at least three amino acids selected from Q176, P177, F178, H179, F180, Y181, F182, P183, or T184.

In another embodiment, a VHH, or a fragment thereof, according to the invention specifically binds to the following amino acids of SEQ ID NO: 14:
1) at least one, or at least two, or at least three amino acids selected from C89, G90, E91, M92, A93, or P94, and
2) at least one, or at least two, or at least three amino acids selected from E140, Q141, W142, W143, E144, D145, C146, R147, T148, S149, or Y150, and
3) at least one, or at least two, or at least three amino acids selected from Q176, P177, F178, H179, F180, Y181, F182, P183, or T184.

In another embodiment, a VHH, or a fragment thereof, according to the invention specifically binds to the following amino acids of SEQ ID NO: 14:
1) at least two, or at least three amino acids selected from C89, G90, E91, M92, A93, or P94, and/or
2) at least two, or at least three amino acids selected from E140, Q141, W142, W143, E144, D145, C146, R147, T148, S149, or Y150, and/or
3) at least two, or at least three amino acids selected from Q176, P177, F178, H179, F180, Y181, F182, P183, or T184.

In another embodiment, a VHH, or a fragment thereof, according to the invention specifically binds to the following amino acids of SEQ ID NO: 14:
1) at least two, or at least three amino acids selected from C89, G90, E91, M92, A93, or P94, and
2) at least two, or at least three amino acids selected from E140, Q141, W142, W143, E144, D145, C146, R147, T148, S149, or Y150, and
3) at least two, or at least three amino acids selected from Q176, P177, F178, H179, F180, Y181, F182, P183, or T184.

In another embodiment, a VHH, or a fragment thereof, according to the invention specifically binds to the following amino acids of SEQ ID NO: 14:
1) at least three amino acids selected from C89, G90, E91, M92, A93, or P94, and/or
2) at least three amino acids selected from E140, Q141, W142, W143, E144, D145, C146, R147, T148, S149, or Y150, and/or
3) or at least three amino acids selected from Q176, P177, F178, H179, F180, Y181, F182, P183, or T184.

In an embodiment, a VHH, or a fragment thereof, according to the invention specifically binds to the following amino acids of SEQ ID NO: 14:
1) at least three amino acids selected from C89, G90, E91, M92, A93, or P94, and
2) at least three amino acids selected from E140, Q141, W142, W143, E144, D145, C146, R147, T148, S149, or Y150, and
3) at least one, or at least two, or at least three amino acids selected from Q176, P177, F178, H179, F180, Y181, F182, P183, or T184.

In another embodiment, the following amino acids of amino acids 25 to 233 of SEQ ID NO: 14 are bound by a VHH, or a fragment thereof, according to the invention: C89, G90, E91, M92, A93, P94, E140, Q141, W142, W143, E144, D145, C146, R147, T148, S149, Y150, Q176, P177, F178, H179, F180, Y181, F182, P183 and T184.

In this context, an amino acid of human FOLR1 may be bound by the VHH or fragment thereof when said amino acid belongs to the epitope of the VHH or fragment thereof.

### First Structural Feature of the VHH or Fragment Thereof

A first structural feature is that a VHH, or a fragment thereof, according to the invention contacts or binds or specifically binds to a region of human FOLR1 comprised within amino acid 25 to 233 of SEQ ID NO: 14. SEQ ID NO: 14 is the human amino acid sequence of human FOLR1. Amino acid 25 to 233 (R25-M233) of SEQ ID NO: 14 is defined as the extracellular domain (without signal peptide M1-T24 and GPI anchor S234 and pro-peptide G235-S257) of human FOLR1 (UniProt Knowledgebase: entry P15328). The region within amino acid 25 to 233 of SEQ ID NO: 14 specifically bound or targeted by the VHH or fragment thereof may be a linear region (i.e. linear epitope or sequential epitope) within said primary amino acid sequence. Alternatively said region may not be linear and may correspond to a conformational epitope. Usually a linear epitope comprises a linear sequence of amino acids that has a length of 5 to 30 amino acids, that is to say that it may have a length of 5, 6, 7, 8, 9, 10, 11, 12, 13, 14, 15, 16, 17, 18, 19, 20, 21, 22, 23, 24, 25, 26, 27, 28, 29 or 30 amino acids. Usually a conformational epitope is characterized by a number of non-consecutive amino acids within amino acid 25 to 233 of SEQ ID NO: 14 that come together in the three-dimensional tertiary structure of the protein and that are contacted by the VHH or fragment thereof.

In the following paragraph dedicated to the second structural feature of the VHH or fragment thereof, linear epitopes and conformational epitope of the VHH or fragment thereof are defined.

### Second Structural Feature of the VHH or Fragment Thereof

A VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) may alternatively or in combination also be further defined by a second structural feature.

A VHH, or a fragment thereof, according to the invention that specifically binds an epitope of human folate receptor alpha (FOLR1), but does neither bind human folate receptor beta (FOLR2) nor human folate receptor gamma (FOLR3) may alternatively or in combination also be further defined by a second structural feature.

A second structural feature is that a VHH, or a fragment thereof, according to the invention contacts or binds or specifically binds to a number of amino acids within amino acid 25 to 233 of SEQ ID NO: 14. These specific amino acids within amino acid 25 to 233 of SEQ ID NO: 14 are further defined below.

In an embodiment, a VHH, or a fragment thereof, according to the invention contacts or binds or specifically binds to at least one of amino acid C89, G90, E91, M92, A93, P94, E140, Q141, W142, W143, E144, D145, C146, R147, T148, S149, Y150, Q176, P177, F178, H179, F180, Y181, F182, P183 and/or T184 of SEQ ID NO: 14. Each combination of 2, 3, 4, 5, 6, 7, 8, 9, 10, 11, 12, 13, 14, 15, 16, 17, 18, 19, 20, 21, 22, 23, 24, 25 or 26 amino acids from the 26 amino acids identified is therefore encompassed to be contacted by the VHH or fragment thereof.

In an embodiment, the linear stretch of amino acids 89-94 of SEQ ID NO: 14 defines a first linear region of hFOLR1, which is contacted, bound or specifically bound by the VHH or fragment thereof. Not each amino acid within this stretch or region may be contacted, bound or specifically bound by the VHH or fragment thereof. In an embodiment, 1, 2, 3, 4, 5 or 6 amino acids of this linear stretch or region is contacted, bound or specifically bound by the VHH or fragment thereof. In an embodiment, this first linear stretch or region is an epitope of the VHH or fragment thereof.

In an embodiment, the linear stretch of amino acids 140-150 of SEQ ID NO: 14 defines a second linear region of hFOLR1, which is contacted, bound or specifically bound by the VHH or fragment thereof. Not each amino acid within this stretch or region needs to be contacted, bound or specifically bound by the VHH or fragment thereof. In an embodiment, 1, 2, 3, 4, 5, 6, 7, 8, 9, 10 or 11 amino acids of this linear stretch or region is contacted, bound or specifically bound by the VHH or fragment thereof. In an embodiment, this second linear stretch or region is a second epitope of the VHH or fragment thereof.

In an embodiment, the linear stretch of amino acids 176-184 of SEQ ID NO: 14 defines a third linear region of hFOLR1, which is contacted, bound or specifically bound by the VHH or fragment thereof. Not each amino acid within this stretch or region needs to be contacted, bound or specifically bound by the VHH or fragment thereof. In an embodiment, 1, 2, 3, 4, 5, 6, 7, 8 or 9 amino acids of this linear stretch or region is contacted, bound or specifically bound by the VHH or fragment thereof. In an embodiment, this third linear stretch or region is a third epitope of the VHH or fragment thereof.

In an embodiment, each of the first, second and third linear stretches or regions defined above is contacted, bound or specifically bound by the VHH or fragment thereof. The combination of these three stretches defines the conformational epitope of the VHH or fragment thereof. Not each amino acid within each of these stretches or regions may be contacted, bound or specifically bound by the VHH or fragment thereof. In an embodiment, 1, 2, 3, 4, 5 or 6 amino acids (or more depending on the length of each stretch) of each of the linear stretches or regions is contacted, bound or specifically bound by the VHH or fragment thereof.

In an embodiment, there is provided a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3), wherein the epitope comprises the linear amino acid stretch or region 89-94, 140-150 and/or 176-184 of SEQ ID NO: 14.

In an embodiment, there is provided a VHH, or a fragment thereof, according to the invention that specifically binds an epitope of human folate receptor alpha (FOLR1), but does neither bind human folate receptor beta (FOLR2) nor human folate receptor gamma (FOLR3), wherein the epitope comprises the linear amino acid stretch or region 89-94, 140-150 and/or 176-184 of SEQ ID NO: 14.

In an embodiment, there is provided a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3), wherein the epitope comprises the combination of linear amino acid stretches of regions 89-94, 140-150 and 176-184 of SEQ ID NO: 14. In an embodiment, there is provided a VHH, or a fragment thereof, according to the invention that specifically binds an epitope of human folate receptor alpha (FOLR1), but does neither bind human folate receptor beta (FOLR2) nor human folate receptor gamma (FOLR3), wherein the epitope comprises the combination of linear amino acid stretches of regions 89-94, 140-150 and 176-184 of SEQ ID NO: 14.

In an embodiment, a VHH, or a fragment thereof, according to the invention contacts or binds or specifically binds to the following amino acids of SEQ ID NO: 14:
at least one, or at least two, or at least three amino acids selected from C89, G90, E91, M92, A93, or P94, and/or
at least one, or at least two, or at least three amino acids selected from E140, Q141, W142, W143, E144, D145, C146, R147, T148, S149, or Y150, and/or at least one, or at least two, or at least three amino acids selected from Q176, P177, F178, H179, F180, Y181, F182, P183, or T184.

In another embodiment, a VHH, or a fragment thereof, according to the invention contacts or binds or specifically binds to the following amino acids of SEQ ID NO: 14:
at least one, or at least two, or at least three, or at least four, or at least five or all six amino acids selected from C89, G90, E91, M92, A93, or P94, and
at least one, or at least two, or at least three, or at least four, or at least five, or at least six or at least seven, or at least eight or at least nine or at least ten or all eleven amino acids selected from E140, Q141, W142, W143, E144, D145, C146, R147, T148, S149, or Y150, and
at least one, or at least two, or at least three, or at least four, or at least five, or at least six, or at least seven, or at least eight, or all nine amino acids selected from Q176, P177, F178, H179, F180, Y181, F182, P183, or T184.

In another embodiment, a VHH, or a fragment thereof, according to the invention contacts or binds or specifically binds to the following amino acids of SEQ ID NO: 14:
1) at least two, or at least three amino acids selected from C89, G90, E91, M92, A93, or P94, and/or
2) at least two, or at least three amino acids selected from E140, Q141, W142, W143, E144, D145, C146, R147, T148, S149, or Y150, and/or
3) at least two, or at least three amino acids selected from Q176, P177, F178, H179, F180, Y181, F182, P183, or T184.

In another embodiment, a VHH, or a fragment thereof, according to the invention contacts or binds or specifically binds to the following amino acids of SEQ ID NO: 14:
1) at least two, or at least three amino acids selected from C89, G90, E91, M92, A93, or P94, and
2) at least two, or at least three amino acids selected from E140, Q141, W142, W143, E144, D145, C146, R147, T148, S149, or Y150, and
3) at least two, or at least three amino acids selected from Q176, P177, F178, H179, F180, Y181, F182, P183, or T184.

In another embodiment, a VHH, or a fragment thereof, according to the invention contacts or binds or specifically binds to the following amino acids of SEQ ID NO: 14:
1) at least three amino acids selected from C89, G90, E91, M92, A93, or P94, and/or
2) at least three amino acids selected from E140, Q141, W142, W143, E144, D145, C146, R147, T148, S149, or Y150, and/or
3) at least three amino acids selected from Q176, P177, F178, H179, F180, Y181, F182, P183, or T184.

In another embodiment, a VHH, or a fragment thereof, according to the invention contacts or binds or specifically binds to the following amino acids of SEQ ID NO: 14:
1) at least three amino acids selected from C89, G90, E91, M92, A93, or P94, and
2) at least three amino acids selected from E140, Q141, W142, W143, E144, D145, C146, R147, T148, S149, or Y150, and
3) or at least three amino acids selected from Q176, P177, F178, H179, F180, Y181, F182, P183, or T184.

In an embodiment, a VHH, or a fragment thereof, according to the invention contacts or binds or specifically binds to the following amino acids of SEQ ID NO: 14: C89, G90, E91, M92, A93 and/or P94.

In an embodiment, a VHH, or a fragment thereof, according to the invention contacts or binds or specifically binds to the following amino acids of SEQ ID NO: 14: E140, Q141, W142, W143, E144, D145, C146, R147, T148, S149 and/or Y150.

In an embodiment, a VHH, or a fragment thereof, according to the invention contacts or binds or specifically binds to the following amino acids of SEQ ID NO: 14: Q176, P177, F178, H179, F180, Y181, F182, P183 and/or T184.

In an embodiment, the following amino acids of amino acids 25 to 233 of SEQ ID NO: 14 are contacted, bound or specifically bound by a VHH, or a fragment thereof, according to the invention: C89, G90, E91, M92, A93, P94, E140, Q141, W142, W143, E144, D145, C146, R147, T148, S149, Y150, Q176, P177, F178, H179, F180, Y181, F182, P183 and T184.

### Third Structural Feature of the VHH or Fragment Thereof

A VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) may alternatively or in combination also be further defined by a third structural feature.

A VHH, or a fragment thereof, according to the invention that specifically binds an epitope of human folate receptor alpha (FOLR1), but does neither bind human folate receptor beta (FOLR2) nor human folate receptor gamma (FOLR3) may alternatively or in combination also be further defined by a third structural feature.

A third structural feature relates to the (full length) amino acid sequence representing a way of defining the family of a VHH, or a fragment thereof, according to the invention. The present invention discloses a family of structurally closely related VHHs or fragments thereof represented by an amino acid sequence comprising, consisting of or essentially consisting of SEQ ID NO: 21 or 22. VHH VHH2 is represented by SEQ ID NO: 21 and VHH3 by SEQ ID NO: 22 (see table 4 below).

Each of SEQ ID NO: 15, 17 and 18 is a part of VHH2 which is conserved amongst the family of the VHH or fragment thereof (see table 4 below).

Each of SEQ ID NO: 16, 19 and 20 is a part of VHH3 which is conserved amongst the family of the VHH or fragment thereof (see table 4 below).

VHHs VHH2 and VHH3 both contact, bind or specifically bind each of the linear stretches or regions of amino acids of SEQ ID NO: 14 as defined earlier herein (i.e. linear amino acid stretch or region 89-94, 140-150 and/or 176-184 of SEQ ID NO: 14). Moreover, both VHH2 and VHH3 have for conformational epitope the combination of stretches or regions of amino acids of SEQ ID NO: 14 as defined earlier herein (i.e. linear amino acid stretches or regions 89-94, 140-150 and 176-184 of SEQ ID NO: 14). VHH2 and VHH3 are two members of a family of VHHs or fragments thereof according to the invention. This family of VHHs or fragments thereof shares at least one of these linear epitopes and/or this conformational epitope. This family of VHHs or fragments thereof has exceptional kinetic characteristics and/or exceptional tissue distribution when used in a combination according to the invention.

TABLE 4
conserved part of VHHs VHH2 or VHH3

| | Amino acid sequence |
|---|---|
| VHH2 | |
| SEQ ID NO: 15 | NHMSWYRQAPGKQRELAAIITSDGNTNYPDS |
| | VKGRFTISRDNAKNTVYLQMNSLKPEDTAVY |
| | YCNTFGRSVAGFYDYW |
| SEQ ID NO: 17 | NHMSWYRQAPGKQRELAAIITSDGNTNYPD |
| SEQ ID NO: 18 | NTFGRSVAGFYDYW |
| SEQ ID NO: 21 | DVQLVESGGGLVQPGGSLRLSCAASESIFSR |
| | NHMSWYRQAPGKQRELAAIITSDGNTNYPDS |
| | VKGRFTISRDNAKNTVYLQMNSLKPEDTAVY |
| | YCNTFGRSVAGFYDYWGQGTQVTVSS |
| VHH3 | |
| SEQ ID NO: 16 | DLAWYRRAPGKQRELAAIITSGGSTNYPDSV |
| | KGRFTISRDNAKNTVYLEMNSLKPEDTAVYY |
| | CNALRPWPQFSDYW |
| SEQ ID NO: 19 | DLAWYRRAPGKQRELAAIITSGGSTNYPD |
| SEQ ID NO: 20 | NALRPWPQFSDYW |
| SEQ ID NO: 22 | DVQLVESGGGLVQAGGSLRLSCAADEMKFSN |
| | YDLAWYRRAPGKQRELAAIITSGGSTNYPDS |
| | VKGRFTISRDNAKNTVYLEMNSLKPEDTAVY |
| | YCNALRPWPQFSDYWGQGTQVTVSS |

This third structural feature of a VHH, or a fragment thereof, according to the invention may be defined in several ways:

In a first embodiment of this third structural feature, the VHH or fragment thereof of the invention is represented by an amino acid sequence that comprises or essentially consists of an amino acid sequence having at least 60% sequence identity (or similarity) with at least one of SEQ ID NO: 15, 16, 17, 18, 19, 20, 21 or 22 or a portion thereof. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a second embodiment of this third structural feature, the VHH or fragment thereof of the invention is represented by an amino acid sequence that comprises or essentially consists of an amino acid sequence having at least 60% sequence identity (or similarity) with at least one of SEQ ID NO: 15, 16, 17, 18, 19, 20, 21 or 22 or a portion thereof and has a length which is ranged from the exact length of SEQ ID NO: 15, 16, 17, 18, 19, 20, 21 or 22 or 1, 2, 3, 4, 5, 6, 7, 8, 9, 10, 15, 20, 25, 30, 35, 40, 45, 50, 55, 60 amino acids longer than the exact length of SEQ ID NO: 15, 16, 17, 18, 19, 20, 21 or 22. For example a tag such as a His tag may be added to the VHH or fragment thereof of the invention. Usually His tag comprises 4, 5, 6, 7, 8, 9, 10 histidines. Alternative tag may be a Hemaglutinin tag (HA-tag): YPYDVPDYA (SEQ ID NO: 37); YPYDVPDYGS (SEQ ID NO: 38) or a cysteine tag (Cys tag). A cysteine tag is a tag that comprises one or several cysteine. Non-limiting examples of cysteine tags are C; GGC; SPSTPPTPSPSTPPC (SEQ ID NO: 39)

The way identity and similarity are assessed is explained in detail in the part dedicated to definition as the end of the description. Usually when identity is defined by reference to a SEQ ID NO, said identity is assessed over the whole SEQ ID NO. However, it is also encompassed by the invention that identity (or similarity) is assessed over a portion (or a fragment) of said sequence. Within this context, a portion may mean at least 50%, 60%, 70%, 80%, 90%, 95% of the length of the SEQ ID NO. The length of the sequence encompassed may still be longer than the length of the SEQ ID NO used to assess the identity (or similarity) (i.e. length being at least 50% of the length of the SEQ ID NO, 60%, 70%, 80%, 90%, the same as the one of the SEQ ID NO even though the identity (or similarity) is assessed over a portion of this SEQ ID NO, or the length being 1, 5, 10, 15, 20, 25, 30, 35, 40, 45, 50, 55, 60 amino acids longer than the exact length of SEQ ID NO: 15, 16, 17, 18, 19, 20, 21 or 22).

In a third embodiment of this third structural feature, the length of the VHH or fragment thereof is from 110 to 130 amino acids or 110, 111, 112, 113, 114, 115, 116, 117, 118, 119, 120, 121, 122, 123, 124, 125, 126, 127, 128, 129 or 130 amino acids. This length does not include the length of a tag, such as a His tag that may be added to the sequence of the VHH or fragment thereof.

In a fourth embodiment of this third structural feature, the VHH or fragment thereof of the invention is represented by an amino acid sequence that comprises or essentially consists of an amino acid sequence having at least 60% sequence identity (or similarity) with at least one of SEQ ID NO: 15, 16, 17, 18, 19, 20, 21 or 22 or a portion thereof and the length of the VHH or fragment thereof is from 80 to 150 amino acids or 90 to 140 or 100 to 130 or 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) with at least of one of these sequences is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In a fifth embodiment of this third structural feature, the VHH or fragment thereof of the invention is represented by an amino acid sequence that comprises or essentially consists of an amino acid sequence having at least 60% sequence identity (or similarity) with SEQ ID NO: 15 or a portion thereof. In an embodiment, the VHH or fragment thereof is represented by an amino acid sequence that comprises or essentially consists of an amino acid sequence having at least 60% sequence identity (or similarity) with SEQ ID NO: 15 or a portion thereof and has a length which is ranged from 78 to 130 amino acids or 78 to 130 or 90 to 120 or 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a sixth embodiment of this third structural feature, the VHH or fragment thereof of the invention is represented by an amino acid sequence that comprises or essentially consists of an amino acid sequence having at least 60% sequence identity (or similarity) with SEQ ID NO: 17 and/or 18 or a portion thereof. In an embodiment, the VHH or fragment thereof is represented by an amino acid sequence that comprises or essentially consists of an amino acid sequence having at least 60% sequence identity (or similarity) with any of SEQ ID NO: 17 and/or 18 or a portion thereof and has a length which is ranged from 14 to 130 amino acids or 20 to 120 or 30 to 110 or 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a seventh embodiment of this third structural feature, the VHH or fragment thereof of the invention is represented by an amino acid sequence that comprises or essentially consists of an amino acid sequence having at least 60% sequence identity (or similarity) with SEQ ID NO: 16 or a portion thereof. In an embodiment, the VHH or fragment thereof is represented by an amino acid sequence that comprises or essentially consists of an amino acid sequence having at least 60% sequence identity (or similarity) with any of SEQ ID NO: 16 or a portion thereof and has a length which is ranged from 76 to 130 amino acids or 76 to 120 or 90 to 110 or 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In an eighth embodiment of this third structural feature, the VHH or fragment thereof of the invention is represented by an amino acid sequence that comprises or essentially consists of an amino acid sequence having at least 60% sequence identity (or similarity) with SEQ ID NO: 19 and/or 20 or a portion thereof. In an embodiment, the VHH or fragment thereof is represented by an amino acid sequence that comprises or essentially consists of an amino acid sequence having at least 60% sequence identity (or similarity) with SEQ ID NO: 19 and/or 20 or a portion thereof and has a length which is ranged from 13 to 150 amino acids or 13 to 140 or 30 to 130 or 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a ninth embodiment of this third structural feature, the VHH or fragment thereof of the invention is represented by an amino acid sequence that comprises or essentially consists of an amino acid sequence having at least 60% sequence identity (or similarity) with SEQ ID NO: 21 and/or 22 or a portion thereof. In an embodiment, the VHH or fragment thereof is represented by an amino acid sequence that comprises or essentially consists of an amino acid sequence having at least 60% sequence identity (or similarity) with SEQ ID NO: 21 and/or 22 or a portion thereof and has a length which is ranged from 110 to 130 amino acids or 100 to 130 or 110 to 120 or 110, 111, 112, 113, 114, 115, 116, 117, 118, 119, 120, 121, 122, 123, 124, 125, 126, 127, 128, 129 or 130 amino acids. In an embodiment, the sequence identity (or similarity) is at least 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%. Portion thereof has been already defined herein.

In a tenth embodiment of this third structural feature, the VHH or fragment thereof, preferably a VHH or a functional fragment thereof is represented by an amino acid sequence that comprises any of SEQ ID NO: 15, 16, 17, 18, 19 and/or 20. In an embodiment, it has a length which is ranged from 13 to 130 amino acids or 20 to 120 or 30 to 121 amino acids. More preferably it is represented by an amino acid sequence that comprises SEQ ID NO: 21 or 22. Even more preferably, it has a length of 118, 119, 120, 121, 122, 123, 124, 125, 126, 127, 128, 129 or 130 amino acids.

In a further embodiment, the VHH or fragment thereof of the invention is according to the ninth embodiment of this third structural feature and contacts or binds or specifically binds to the following amino acids of SEQ ID NO: 14:
at least one, or at least two, or at least three amino acids selected from C89, G90, E91, M92, A93, or P94, and/or
at least one, or at least two, or at least three amino acids selected from E140, Q141, W142, W143, E144, D145, C146, R147, T148, S149, or Y150, and/or
at least one, or at least two, or at least three amino acids selected from Q176, P177, F178, H179, F180, Y181, F182, P183, or T184.

In a further embodiment, the VHH or fragment thereof of the invention is according to the tenth embodiment of this third structural feature and contacts or binds or specifically binds to the following amino acids of SEQ ID NO: 14:
at least one, or at least two, or at least three amino acids selected from C89, G90, E91, M92, A93, or P94, and/or
at least one, or at least two, or at least three amino acids selected from E140, Q141, W142, W143, E144, D145, C146, R147, T148, S149, or Y150, and/or
at least one, or at least two, or at least three amino acids selected from Q176, P177, F178, H179, F180, Y181, F182, P183, or T184.

In an embodiment, a VHH, or a fragment thereof, according to the invention specifically binds an epitope of human folate receptor alpha (FOLR1), but does neither bind murine FOLR1 nor human folate receptor beta (FOLR2) nor human folate receptor gamma (FOLR3), wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

In an embodiment, a VHH, or a fragment thereof, according to the invention specifically binds an epitope of human folate receptor alpha (FOLR1), but does neither bind human folate receptor beta (FOLR2) nor human folate receptor gamma (FOLR3), wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

In an embodiment, the sequence identity (or similarity) is at least 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a VHH, or a fragment thereof, according to the invention specifically binds an epitope of human folate receptor alpha (FOLR1), but does neither bind murine FOLR1 nor human folate receptor beta (FOLR2) nor human folate receptor gamma (FOLR3), wherein the epitope comprises the linear amino acid stretch or region 89-94, 140-150 and/or 176-184 of SEQ ID NO: 14 and wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

In an embodiment, a VHH, or a fragment thereof, according to the invention specifically binds an epitope of human folate receptor alpha (FOLR1), but does neither bind human folate receptor beta (FOLR2) nor human folate receptor gamma (FOLR3), wherein the epitope comprises the linear amino acid stretch or region 89-94, 140-150 and/or 176-184 of SEQ ID NO: 14 and wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

In an embodiment, the sequence identity (or similarity) is at least 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

In an embodiment, a VHH, or a fragment thereof, according to the invention specifically binds an epitope of human folate receptor alpha (FOLR1), but does neither bind murine FOLR1 nor human folate receptor beta (FOLR2) nor human folate receptor gamma (FOLR3), wherein the epitope comprises the combination of linear amino acid stretches or regions 89-94, 140-150 and 176-184 of SEQ ID NO: 14, and wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

In an embodiment, a VHH, or a fragment thereof, according to the invention specifically binds an epitope of human folate receptor alpha (FOLR1), but does neither bind human folate receptor beta (FOLR2) nor human folate receptor gamma (FOLR3), wherein the epitope comprises the combination of linear amino acid stretches or regions 89-94, 140-150 and 176-184 of SEQ ID NO: 14, and wherein the VHH or fragment thereof is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence.

In an embodiment, the sequence identity (or similarity) is at least 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99% or 100%.

### Fourth Structural Feature

A VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) may also be alternatively or in combination further defined by a fourth structural feature.

A VHH, or a fragment thereof, according to the invention that specifically binds an epitope of human folate receptor alpha (FOLR1), but does neither bind human folate receptor beta (FOLR2) nor human folate receptor gamma (FOLR3) may also be alternatively or in combination further defined by a fourth structural feature.

In a fourth structural feature, the VHH or fragment thereof of the invention is represented by an amino acid sequence that comprises at least one combination of CDR sequences chosen from the group comprising: a CDR1 region having SEQ ID NO: 23, a CDR2 region having has SEQ ID NO: 24, and a CDR3 region having SEQ ID NO: 25, and/or a CDR1 region having SEQ ID NO: 30 a CDR2 region having has SEQ ID NO: 31, and a CDR3 region having SEQ ID NO: 32 (see table 5).

Thus, in particular embodiments, the present invention provides heavy chain variable domains comprising the heavy chain antibodies with the (general) structure or which is derived therefrom:

FR1-CDR1-FR2-CDR2-FR3-CDR3-FR4

in which FR1 to FR4 refer to framework regions 1 to 4, respectively, and in which CDR1 to CDR3 refer to the complementarity determining regions 1 to 3, respectively, and are as further defined herein.

SEQ ID NO: 21 and 22 (see table 5) give the amino acid sequences of heavy chain variable domains that have been raised against human FOLR1.

TABLE 5
CDR and FR of VHHs VHH2 and VHH3 (using the
IMGT nomenclature as defined later on herein in
the general part of the description dedicated
to the general definition of the invention)

| | VHH2 (SEQ ID NO: 21) |
|---|---|
| CDR of VHH2 | |
| CDR1 (SEQ ID NO: 23) | ESIFSRNH |
| CDR2 (SEQ ID NO: 24) | ITSDGNT |
| CDR3 (SEQ ID NO: 25) | NTFGRSVAGFYDY |
| FR of VHH2 | |
| FR1 (SEQ ID NO: 26) | DVQLVESGGGLVQPGGSLRLSCAAS |
| FR2 (SEQ ID NO: 27) | MSWYRQAPGKQRELAAI |
| FR3 (SEQ ID NO: 28) | NYPDSVKGRFTISRDNAKNTVYLQM |
| | NSLKPEDTAVYYC |
| FR4 (SEQ ID NO: 29) | WGQGTQVTVSS |
| | VHH3 (SEQ ID NO: 22) |
| CDR of VHH3 | |
| CDR1 (SEQ ID NO: 30) | EMKFSNYD |
| CDR2 (SEQ ID NO: 31) | ITSGGST |
| CDR3 (SEQ ID NO: 32) | NALRPWPQFSDY |
| FR of VHH3 | |
| FR1 (SEQ ID NO: 33) | DVQLVESGGGLVQAGGSLRLSCAAD |
| FR2 (SEQ ID NO: 34) | LAWYRRAPGKQRELAAI |
| FR3 (SEQ ID NO: 35) | NYPDSVKGRFTISRDNAKNTVYLEM |
| | NSLKPEDTAVYYC |
| FR4 (SEQ ID NO: 36) | WGQGTQVTVSS |

It should be noted that the invention is not limited as to the origin of the VHH or fragment thereof (or of the nucleotide sequences to express these), nor as to the way that the VHH or fragment thereof, preferably VHH or fragments thereof or nucleotide sequences disclosed herein are (or have been) generated or obtained. Thus, the VHH or fragment thereof disclosed herein may be naturally occurring amino acid sequences (from any suitable species) or synthetic or semi-synthetic amino acid sequences. Methods for isolating and methods of producing VHHs or fragments thereof as well as nucleic acid molecule encoding the VHH or fragment thereof, construct comprising these nucleic acid molecule and cells comprising these constructs are disclosed in detail in the definition part at the end of the description.

In a specific but non-limiting aspect of the invention, the amino acid sequence of the VHH or fragment thereof is a naturally occurring immunoglobulin sequence (from any suitable species) or a synthetic or semi-synthetic immunoglobulin sequence, including but not limited to “humanized” immunoglobulin sequences (such as partially or fully humanized mouse or rabbit immunoglobulin sequences, and in particular partially or fully humanized VHH sequences), “camelized” immunoglobulin sequences, as well as immunoglobulin sequences that have been obtained by techniques such as affinity maturation (for example, starting from synthetic, random or naturally occurring immunoglobulin sequences), CDR grafting, veneering, combining fragments derived from different immunoglobulin sequences, PCR assembly using overlapping primers, and similar techniques for engineering immunoglobulin sequences well known to the skilled person; or any suitable combination of any of the foregoing. Also, a VHH, or a fragment thereof, according to the invention may be suitably humanized, as further described herein, so as to provide one or more further (partially or fully) humanized amino acid sequences of the invention.

In an embodiment, the VHH or fragment thereof of the invention is derived from the VHH or fragment thereofs described above using CDR grafting. Preferred VHHs or fragments thereof comprise a:
CDR1 region having SEQ ID NO: 23, a CDR2 region having has SEQ ID NO: 31, and a CDR3 region having SEQ ID NO: 25,
CDR1 region having SEQ ID NO: 23, a CDR2 region having has SEQ ID NO: 31, and a CDR3 region having SEQ ID NO: 32,
CDR1 region having SEQ ID NO: 23, a CDR2 region having has SEQ ID NO: 24, and a CDR3 region having SEQ ID NO: 32,
CDR1 region having SEQ ID NO: 30 a CDR2 region having has SEQ ID NO: 24, and a CDR3 region having SEQ ID NO: 32,
CDR1 region having SEQ ID NO: 30 a CDR2 region having has SEQ ID NO: 24, and a CDR3 region having SEQ ID NO: 25, or
CDR1 region having SEQ ID NO: 30 a CDR2 region having has SEQ ID NO: 31, and a CDR3 region having SEQ ID NO: 25.

Each of these VHHs or fragments thereof may have the FR of VHH2 or of VHH3 as identified in table 5. However, distinct FR may be present.

In another embodiment, the VHH or fragment thereof of the invention is derived from the VHH or fragment thereof described above using CDR grafting. Preferred VHHs or fragments thereof comprise a:
CDR1 region having SEQ ID NO: 23 and a CDR3 region having SEQ ID NO: 25,
CDR1 region having SEQ ID NO: 23 and a CDR3 region having SEQ ID NO: 32,
CDR1 region having SEQ ID NO: 30 and a CDR3 region having SEQ ID NO: 32,
CDR1 region having SEQ ID NO: 30 and a CDR3 region having SEQ ID NO: 25,
Each of these VHHs or fragments thereof may comprise a CDR2 region from another VHH and may have the FR of VHH2 or of VHH3 as identified in table 5. However, distinct FR may be present.

In another embodiment, the VHH or fragment thereof of the invention is derived from the VHH or fragment thereof described above using CDR grafting. Preferred VHHs or fragments thereof comprise a:
CDR1 region having SEQ ID NO: 23 and a CDR2 region having SEQ ID NO: 24,
CDR1 region having SEQ ID NO: 23 and a CDR2 region having SEQ ID NO: 31,
CDR1 region having SEQ ID NO: 30 and a CDR2 region having SEQ ID NO: 31,
CDR1 region having SEQ ID NO: 30 and a CDR2 region having SEQ ID NO: 24,
Each of these VHHs or fragments thereof may comprise a CDR3 region from another VHH and may have the FR of VHH2 or of VHH3 as identified in table 5. However, distinct FR may be present.

In another embodiment, the VHH or fragment thereof of the invention is derived from the VHH or fragment thereofs described above using CDR grafting. Preferred VHHs or fragments thereof comprise a:
CDR2 region having SEQ ID NO: 24 and a CDR3 region having SEQ ID NO: 25,
CDR2 region having SEQ ID NO: 24 and a CDR3 region having SEQ ID NO: 32,
CDR2 region having SEQ ID NO: 31 and a CDR3 region having SEQ ID NO: 32,
CDR2 region having SEQ ID NO: 31 and a CDR3 region having SEQ ID NO: 25,
Each of these VHHs or fragments thereof may comprise a CDR1 region from another VHH and may have the FR of VHH2 or of VHH3 as identified in table 5. However, distinct FR may be present.

In another embodiment, the VHH or fragment thereof of the invention is derived from the VHH or fragment thereof described above using CDR grafting. Preferred VHHs or fragments thereof comprise a: CDR1 region having SEQ ID NO: 23 or 30 and may comprise a CDR2 and CDR3 region from another VHH and may have the FR of VHH2 or of VHH3 as identified in table 5. However, distinct FR may be present.

In another embodiment, the VHH or fragment thereof of the invention is derived from the VHH or fragment thereof described above using CDR grafting. Preferred VHHs or fragments thereof comprise a:
CDR2 region having SEQ ID NO: 24 or 31 and may comprise a CDR1 and CDR3 region from another VHH and may have the FR of VHH2 or of VHH3 as identified in table 5. However, distinct FR may be present.

In another embodiment, the VHH or fragment thereof of the invention is derived from the VHH or fragment thereof described above using CDR grafting. Preferred VHHs or fragments thereof comprise a:
CDR3 region having SEQ ID NO: 25 or 19 and may comprise a CDR1 and CDR2 region from another VHH and may have the FR of VHH2 or of VHH3 as identified in table 5. However, distinct FR may be present.

Similarly, when an amino acid sequence comprises a synthetic or semi-synthetic sequence (such as a partially humanized sequence), said sequence may optionally be further suitably humanized, again as described herein, so as to provide one or more further (partially or fully) humanized amino acid sequences as disclosed herein. At the end of the description, a more detailed definition of “agonist” “antagonist”, “variants”, “posttranslational structural characterization” is provided.

In particular, humanized VHHs may be represented by amino acid sequences in which at least one amino acid residue is present (and in particular, in at least one of the framework residues) that is and/or that corresponds to a humanizing substitution. In addition, or alternatively, other potentially useful humanizing substitutions can be ascertained by comparing the sequence of the framework regions of a naturally occurring VHH sequence with the corresponding framework sequence of one or more closely related human V_{H} sequences, after which one or more of the potentially useful humanizing substitutions (or combinations thereof) thus determined can be introduced into said VHH sequence (in any manner known per se, as further described herein) and the resulting humanized VHH sequences or functional fragments thereof can be tested for affinity for the target, for stability, for ease and level of expression, and/or for other desired properties. In this way, by means of a limited degree of trial and error, other suitable humanizing substitutions (or suitable combinations thereof) can be determined by the skilled.

In an embodiment, a VHH, or a fragment thereof, according to the invention is represented by a first, second, third and/or fourth structural feature as identified herein. Alternatively or in combination with said structural feature, the VHH or fragment thereof is characterized by a functional feature which is to specifically bind human folate receptor alpha (FOLR1), but (preferably not murine FOLR1), not human folate receptor beta (FOLR2) and not human folate receptor gamma (FOLR3). In an embodiment, the VHH or fragment thereof is characterized by a functional feature which is to specifically bind human folate receptor alpha (FOLR1), but (preferably not murine FOLR1), not human folate receptor beta (FOLR2) and not human folate receptor gamma (FOLR3).

In a preferred embodiment, a combination according to the invention comprises:
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the linear amino acid stretch or region 89-94, 140-150 and/or 176-184 of SEQ ID NO: 14; and a labelled compound comprising iodine-131 and GMIB,
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the linear amino acid stretch or region 89-94, 140-150 and/or 176-184 of SEQ ID NO: 14; and a labelled compound comprising DOTA or DTPA and lutetium-177, preferably wherein the labelled compound can be represented by ¹⁷⁷Lu-DOTA-PEG₇-Tz or ¹⁷⁷Lu-DTPA-PEG₇-Tz,
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the linear amino acid stretch or region 89-94, 140-150 and/or 176-184 of SEQ ID NO: 14; and a labelled compound comprising DOTA and actinium-225, preferably wherein the labelled compound can be represented by ²²⁵Ac-DOTA-PEG₇-Tz, or
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the linear amino acid stretch or region 89-94, 140-150 and/or 176-184 of SEQ ID NO: 14; and a labelled compound comprising technetium-99m.

In a preferred embodiment, a combination according to the invention comprises:
a VHH, or a fragment thereof, according to the invention, that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the combination of linear amino acid stretches or regions 89-94, 140-150 and 176-184 of SEQ ID NO: 14; and a labelled compound comprising iodine-131 and GMIB,
a VHH, or a fragment thereof, according to the invention, that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the combination of linear amino acid stretches or regions 89-94, 140-150 and 176-184 of SEQ ID NO: 14; and a labelled compound comprising DOTA or DTPA and lutetium-177, preferably wherein the labelled compound can be represented by ¹⁷⁷Lu-DOTA-PEG₇-Tz or ¹⁷⁷Lu-DTPA-PEG₇-Tz,
a VHH, or a fragment thereof, according to the invention, that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the combination of linear amino acid stretches or regions 89-94, 140-150 and 176-184 of SEQ ID NO: 14; and a labelled compound comprising DOTA and actinium-225, preferably wherein the labelled compound can be represented by ²²⁵Ac-DOTA-PEG₇-Tz, or
a VHH, or a fragment thereof, according to the invention, that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein the epitope comprises the combination of linear amino acid stretches or regions 89-94, 140-150 and 176-184 of SEQ ID NO: 14; and a labelled compound comprising technetium-99m.

In a preferred embodiment, a combination according to the invention comprises:
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence; and a labelled compound comprising iodine-131 and GMIB,
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence; and a labelled compound comprising DOTA or DTPA and lutetium-177, preferably wherein the labelled compound can be represented by ¹⁷⁷Lu-DOTA-PEG₇-Tz or ¹⁷⁷Lu-DTPA-PEG₇-Tz,
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence; and a labelled compound comprising DOTA and actinium-225, preferably wherein the labelled compound can be represented by ²²⁵Ac-DOTA-PEG₇-Tz, or,
a VHH, or a fragment thereof, according to the invention that specifically binds an epitope of human folate receptor alpha (FOLR1), but does neither bind murine FOLR1 nor human folate receptor beta (FOLR2) nor human folate receptor gamma (FOLR3) (or a VHH, or a fragment thereof, according to the invention specifically binding human FOLR1, but neither binding human FOLR2 nor human FOLR3), wherein said antibody fragment is represented by an amino acid sequence that comprises an amino acid sequence having at least 63% sequence identity with at least one of SEQ ID NO: 21, 22, 15, 16, 17, 18, 19, or 20 over the full length of said sequence or over at least 50% of the length of said sequence; and a labelled compound comprising technetium-99m.

### Human Epidermal Growth Factor Receptor 2 (HER2)

In embodiments, a VHH, or a fragment thereof, according to the invention is particularly suited for binding to human epidermal growth factor receptor 2. Example 8 demonstrates that TCO-conjugated HER2-binding VHHs can be used in pre-targeting applications. Preferably, the VHH or fragment thereof does not compete with the monoclonal antibody Trastuzumab (Herceptin®) or the monoclonal antibody Pertuzumab (Perjeta®) for binding to HER2, as determined using a suitable competition assay.

In embodiments, a VHH, or a fragment thereof, according to the invention comprises one of the CDR combinations chosen from the group comprising:
a CDR1 region having SEQ ID NO: 40, a CDR2 region having SEQ ID NO: 41, and a CDR3 region having SEQ ID NO: 42, and/or
a CDR1 region having SEQ ID NO: 43, a CDR2 region having SEQ ID NO: 44, and a CDR3 region having SEQ ID NO: 45.

In embodiments, a VHH, or a fragment thereof, according to the invention has at least 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99 or 100% amino acid sequence identity with at least one of the amino acid sequences of SEQ ID NO: 46 or 47.

In embodiments, a VHH, or a fragment thereof, according to the invention comprises amino acid sequences SEQ ID NO: 46 or 47.

In embodiments, a VHH, or a fragment thereof, according to the invention comprises or consists of any of amino acid sequences SEQ ID NO: 48-88. In embodiments, a VHH, or a fragment thereof, according to the invention has at least 60% amino acid identity with at least one of amino acid sequences SEQ ID NO: 48-88. In embodiments, a VHH, or a fragment thereof, according to the invention has at least 70% amino acid identity with at least one of amino acid sequences SEQ ID NO: 48-88. In embodiments, a VHH, or a fragment thereof, according to the invention has at least 80% amino acid identity with at least one of amino acid sequences SEQ ID NO: 48-88. In embodiments, a VHH, or a fragment thereof, according to the invention has at least 90% amino acid identity with at least one of amino acid sequences SEQ ID NO: 48-88. In embodiments, a VHH, or a fragment thereof, according to the invention has at least 95% amino acid identity with at least one of amino acid sequences SEQ ID NO: 48-88.

In embodiments, a VHH, or a fragment thereof, according to the invention is present in a monovalent format.

In embodiments, a VHH, or a fragment thereof, according to the invention is devoid of a cysteine-containing tag, preferably a GGC-tag.

In embodiments, a VHH, or a fragment thereof, according to the invention is non-lifetime extended.

In embodiments, a VHH, or a fragment thereof, according to the invention is devoid of a carboxy-terminal polypeptide tag, preferably wherein said VHH or fragment thereof is untagged.

### Combinations Comprising Antibodies and Antibody Fragments Binding FAP or FOLR1

In an aspect of the invention, there is provided a combination for use in the treatment and/or diagnosis of a cancer, comprising:
an antibody, or a fragment thereof, comprising a click group; and
a labelled compound, comprising a label and a click group;

wherein said antibody, or fragment thereof, is able to specifically bind human and/or murine FAP, or is able to specifically bind an epitope of human folate receptor alpha (FOLR1, but does neither bind human folate receptor beta (FOLR2) nor human folate receptor gamma (FOLR3);

wherein said use comprises a first administration of said antibody, or fragment thereof, and a subsequent administration of said labelled compound to a subject in need thereof;

wherein said click group comprised in said antibody, or fragment thereof, is able to undergo a click reaction in said subject with said click group comprised in said labelled compound.

All preferred aspects and embodiments relating to combinations according to the invention may be applied mutatis mutandis to combinations according to the combinations this aspect. For example, the preferred click groups comprised in a VHH, or a fragment thereof, which is part of a combination according to the invention are also preferred click groups comprised in the antibody, or fragment thereof, described above. Furthermore, all preferred sequences and other structural and functional variations of a VHH, or a fragment thereof, which may be part of a combination according to the invention, may also apply to the antibody, or fragment thereof. Evidently, other preferred features of a combination according to the invention, relating to elements such as, but not being limited to, the cancer, the labelled compound, the first administration, the second administration and the click reaction, apply mutatis mutandis to the combinations described above.

### Composition

In a further aspect there is provided a composition comprising, consisting of, or consisting essentially of a VHH, or a fragment thereof, according to the invention. Such a composition is called a composition according to the invention in the context of this application. All preferred embodiments relating to a VHH, or a fragment thereof, according to the invention, optionally comprised in a combination according to the invention, apply mutatis mutandis to such a composition. In a combination according to the invention, the VHH or fragment thereof may be comprised in a composition according to the invention.

In another aspect there is provided a composition comprising, consisting of, or consisting essentially of a labelled compound as used in a combination according to the invention. In a combination according to the invention, the labelled compound may be comprised in such a composition.

The compositions above may comprises an excipient. The excipient should be acceptable for diagnostic and/or therapeutic purpose. In an embodiment the composition is a pharmaceutical composition. In another embodiment, the composition is a diagnostic composition. It is also encompassed by the invention that the composition is a pharmaceutical and diagnostic composition. Suitable formulations of the invention are disclosed in the definition part at the end of the description.

The compositions may comprise one or more acceptable carrier.

The compositions may comprise at least one other compound.

The pharmaceutical compositions as envisaged herein can be used in the prevention and/or treatment of a cancer.

### Diagnostic Use of a Combination

In a further aspect, there is provided a method wherein a combination according to the invention is used to assess expression of an antigen, preferably human FAP or human FOLR1, in a subject or in an isolated sample of said subject. This method may comprise the following steps:
a) providing a combination according to the invention,
b) a first administration of said VHH, or fragment thereof, comprised in said combination and a subsequent administration of said labelled compound comprised in said combination to a subject in need thereof or to an isolated sample of a subject,
c) assessing the expression of the antigen in said subject or in said isolated sample of said subject.

This method may be called a diagnostic method. This method may be an in vitro or an in vivo method. This method may allow the localization of the expression of the antigen, preferably human FAP or human FOLR1, in a subject or in an isolated sample of said subject and may allow the prediction and/or prognosis of a certain disease and/or disorder and/or condition in said subject. In an embodiment, this method may be a stratification method to identify patients that are likely to respond to a particular treatment such as cancer treatment or wound healing treatment or fibrosis treatment. Therefore, in a further aspect, there is provided a method wherein the combination according to the invention is used to stratify the subject and assess whether the subject will be likely to respond to a particular treatment such as cancer treatment or wound healing treatment or fibrosis treatment. This method may comprise the following steps:
a) providing a combination according to the invention,
b) a first administration of said VHH, or fragment thereof, comprised in said combination and a subsequent administration of said labelled compound comprised in said combination to a subject in need thereof or to an isolated sample of a subject,
c) assessing the expression of the antigen in said subject or in said isolated sample of said subject and
d) deciding whether the subject is likely to be responsive to a medical treatment.

Preferably, the label comprised in the combination is a radionuclide. The subject is preferably a human being. Detailed information is disclosed in the definition part at the end of the description in order to produce/provide and in order to administer a combination according to the invention. The administration of a combination for diagnostic or therapeutic purpose is similar. A method according to this aspect may be an in vitro, ex vivo method.

In an embodiment, a screening dose or a biomarker dose is administered to a subject or to an isolated sample of said subject. Detailed definitions are provided later on especially by comparison to the definition of a therapeutic dose.

In an embodiment of the diagnostic method, the labelled compound comprised in a combination according to the invention comprises [I-131]GMIB, or comprises a DTPA or DOTA linker and a lutetium-177 radionuclide, or comprises a technetium-99m radionuclide.

The assessment of the expression of an antigen, preferably human FAP or human FOLR1, in the subject is preferably carried out using imaging as disclosed in the part dedicated to definition at the end of the description. Alternatively, the assessment of the expression of an antigen, preferably human FAP or human FOLR1, in the subject is preferably carried out using an isolated sample of the subject. Within the context of the invention, an isolated sample of a subject may be a tissue or a liquid sample from said subject. A liquid may be serum. An isolated sample from a patient may be called a biopsy or a tumour biopsy.

### Therapeutic Use of a Combination

In an embodiment, a combination according to the invention, and correspondingly a VHH or fragment thereof according to the invention, for use in the treatment and/or diagnosis of a solid cancer, preferably wherein said solid cancer is derived from an epithelial cell or tissue.

In an embodiment, a combination according to the invention comprises a label which is a molecule to be delivered to a cell, a tissue, organ expressing or over-expressing the corresponding antigen, preferably human FAP or human FOLR1. The molecule may be a peptide or a small molecule, a nucleic acid. A peptide may be a cytokine. A small molecule may be a chemotherapeutic. An entity may be a cell such as a CAR-T cell, a CAR-NK cell, a BITE or a LITE. This compound or a composition comprising it may be a medicament for treating a disease or condition associated with the expression or the over-expression of the antigen, preferably FAP or FOLR1.

Any moiety, molecule or medicament known to act on a cell, tissue, organ expressing the antigen, preferably human FAP or human FOLR1, is potentially encompassed by the present invention and could be a label comprised in a combination according to the invention.

In embodiments, a combination according to the invention is for use in the treatment of a cancer. In a further embodiment, said cancer is associated with an expression of human FAP on a cancer or a tumour cell or a metastasized lesion. The cancer treated may be metastatic, preferably wherein a metastatic cell is found in the brain, bones, liver, lung. A cancer associated with expression of FAP may be any of a leukemia, bone, uterus, pancreas, GEP-NET (gastroenteropancreatic neuroendocrine tumour), skin, muscle, brain, breast, colorectal, esophageal, gastric, liver, lung, NSCLC (non small cell lung cancer) ovarian, parathyroid, renal cancer cells, CUP (cancer of unknown primary), prostate, small intestine, CCC (Cholangiocellular Carcinoma), sarcoma, (Puré et al 2018, Oncogene August; 37 (32): 4343-4357 and Frederik Giesel et al J Nucl Med May 1, 2019 vol. 60 no. supplement 1 Abstract 289). However, the invention is not limited to these types of cancer. As soon as a subject is suspected to have a cancer cell expressing or overexpressing human FAP, the combination according to the invention may be used.

In an alternative further embodiment, said cancer is associated with an expression of human FOLR1. In an embodiment, the label comprised in the combination is a molecule to be delivered to the central nervous system (CNS). The molecule preferably does not cross the brain blood barrier (BBB) on its own. The molecule may be a peptide or a small molecule, a nucleic acid. A peptide may be a cytokine. A small molecule may be a chemotherapeutic. An entity may be a cell such as a CAR-T cell, a CAR-NK cell, a BITE or a LITE. This compound may be a medicament for treating a disease or condition associated with the brain or wherein the medicament is for treating a disease or condition wherein an alteration of a brain activity will impact another organ of the subject.

More preferably, the molecule is a medicament acting in the CNS, preferably acting in the brain. Even more preferably, the molecule crosses the brain blood barrier (BBB) and/or the blood-cerebrospinal fluid barrier (BCSFB) via transport via the human FOLR1; a mechanism called receptor mediated transcytosis (RMT). Any medicament known to act in the CNS or in the brain is potentially encompassed by the present invention.

A disease or a condition or a disorder associated with the CNS or the brain may be any disease, condition or disorder known to the skilled person as being associated with the CNS or the brain. Such disease, condition or disorder may reflect an altered CNS activity or an altered brain activity. Examples of such disease, condition or disorder include: epilepsy, autism spectrum disorders, autism, altered food intake, altered heat regulation, altered pain sensation, chronic pain, depression, migraines, hearing loss, bipolar disorders, Alzheimer's disease, schizophrenia, brain injury, blindness, stroke, Parkinson's disease, multiple sclerosis, spinal cord injury, amyotrophic lateral sclerosis.

In an embodiment, the cancer is associated with an expression of human FOLR1 on a cancer or a tumour cell or a metastasized lesion. The cancer treated may be metastatic, preferably wherein a metastatic cell is found in the brain. This is an attractive embodiment as the labelled compound is able to cross the BBB. A cancer associated with expression of FOLR1 may be any of an ovarian, endometrial, brain, lung, adrenal carcinoma, head and neck, breast, stomach, colon-rectum cancer. However, the invention is not limited to these types of cancer. As soon as a subject is suspected to have a cancer cell expressing or overexpressing human FOLR1, a combination according to the invention may be used.

In an embodiment, the subject has been first diagnosed using a combination according to the invention before being treated with a combination comprising the same or a different label, preferably a radionuclide. The identity of the radionuclide may not be the same in diagnostic and therapy applications. In an embodiment of this therapeutic method or use, the labelled compound comprised in a combination according to the invention comprises [I-131]GMIB, or comprises a DTPA or DOTA linker and a lutetium-177 radionuclide, or comprises a technetium-99m radionuclide.

Within the context of the invention, a disease or condition or disorder has been prevented or treated when the administration of a combination according to the invention has been carried out and has resulted:
in the improvement of at least one symptom associated with said disease or condition or disorder and/or
in the improvement of at least one parameter associated with said disease or condition or disorder.

The improvement may be observed at least one day, two days, three days, four days, five days, six days, one week after the compound, respective labelled compound has been administrated. Alternatively, the improvement may be observed at least one month, six months after the administration of the combination.

Envisaged doses and administration modes are further disclosed in the definition part at the end of the description.

The (therapeutic) use of a combination according to the invention leads to or exhibits an anti-cancer activity when at least one of the following is fulfilled:
it can kill a tumour cell, a cancer cell and/or a CAF that expresses the antigen, preferably human FAP or human FOLR1,
It can reduce or slow the growth and/or proliferation of such a tumour cell or cancer cell.
It can reduce the size of a primary tumour or of a metastatic lesion,
It can delay the occurrence of metastases and/or of tumour cell migration,
It can delay the increase of a tumour weight or growth, and
It can extend patient survival of at least one month, several months or more (compared to those not treated or treated with a control or compared with the subject at the onset of the treatment).

An anti-cancer activity may have been identified or determined when the number of viable cancer cells, and/or viable tumour cells after the administration of the combination according to the invention is less than 90%, less than 80%, less than 70%, less than 60%, less than 50%, less than 40%, less than 30%, less than 20%, less than 10% of the number of initial viable cancer cells and/or initial viable tumour cells.

An anti-cancer activity may have been identified or determined when the size of a primary tumour and/or the size of a metastatic lesion after the administration of the combination according to the invention is less than 90%, less than 80%, less than 70%, less than 60%, less than 50%, less than 40%, less than 30%, less than 20%, less than 10% of the size of said primary tumour and/or of the size of said metastatic lesion.

Tumour cell death may be assessed by measurement of radiolabelled Annexin A5, a molecular imaging agent to measure cell death in vitro, and non-invasively in patients with cancer such as ICH (Schutters K. et al., Apoptosis 2010; de Saint-Hubert M. et al., Methods 48:178, 2009). ICH has been defined in the definition part at the end of the description.

Tumour growth may be inhibited at least 5%, 10%, 20%, 30%, 40%, 50%, 55%, 60%, 65%, 70% or 75%, or more. Tumour growth may be assessed using techniques known to the skilled person. Tumour growth may be assessed using MRI (Magnetic Resonance Imaging) or CT (Computer Tomography).

In certain embodiments, tumour weight increase or tumour growth may be inhibited at least 20%, 30%, 40%, 50%, 55%, 60%, 65%, 70% or 75%, or more. Tumour weight or tumour growth may be assessed using techniques known to the skilled person. The detection of tumour growth or the detection of the proliferation of tumour cells may be assessed in vivo by measuring changes in glucose utilization by positron emission tomography with the glucose analogue 2-[18F]-fluor-2-deoxy-D-glucose (FDG-PET) or [18F]-′3-fluoro-′3-deoxy-L-thymidine (FLT-PET). An ex vivo alternative may be staining of a tumour biopsy with Ki67.

A delay in occurrence of metastases and/or of tumour cell migration may be a delay of at least one week, one month, several months, one year or longer. The presence of metastases may be assessed using MRI, CT or Echography or techniques allowing the detection of circulating tumour cells (CTC). Examples of the latter tests are CellSearch CTC test (Veridex), an EpCam-based magnetic sorting of CTCs from peripheral blood.

In certain embodiments, tumour growth may be delayed or inhibited at least one day, two days, three days, four days, five days, six days or one week, two weeks, three weeks, one month, two months or more. In a certain embodiment, an occurrence of metastases is delayed at least one week, two weeks, three weeks, four weeks, one months, two months, three months, four months, five months, six months or more.

The combination according to the invention exerts its anti-cancer activity through the mechanism of radiotoxicity once it is bound to a cancer or tumour cell or metastatic lesion or CAF expressing the antigen, preferably human FAP or human FOLR1.

Tumour cell, cancer cell, lesion, metastatic lesion and dose of the combination have been defined in the section entitled definition.

In a further aspect, there is provided a method for the prevention and/or treatment of a disease and/or disorder and/or condition comprising administering to a subject in need thereof, a combination according to the invention. All features of this method have been defined earlier herein.

### Definitions

The following terms or definitions are provided solely to aid in the understanding of the invention. Unless specifically defined herein, all terms used herein have the same meaning as they would to one skilled in the art of the present invention. Practitioners are particularly directed to Sambrook et al., Molecular Cloning: A Laboratory Manual, 2^{nd} ed., Cold Spring Harbor Press, Plainsview, New York (1989); and Ausubel et al., Current Protocols in Molecular Biology (Supplement 47), John Wiley & Sons, New York (1999), for definitions and terms of the art. The definitions provided herein should not be construed to have a scope less than understood by a person of ordinary skill in the art.

Unless indicated otherwise, all methods, steps, techniques and manipulations that are not specifically described in detail can be performed and have been performed in a manner known per se, as will be clear to the skilled person. Reference is for example again made to the standard handbooks, to the general background art referred to above and to the further references cited therein.

As used herein, the singular forms ‘a’, ‘an’, and ‘the’ include both singular and plural referents unless the context clearly dictates otherwise.

The terms ‘comprising’, ‘comprises’ and ‘comprised of’ as used herein are synonymous with ‘including’, ‘includes’ or ‘containing’, ‘contains’, and are inclusive or open-ended and do not exclude additional, non-recited members, elements or method steps. The expression “essentially consists of” used in the context of a product or a composition (“a product essentially consisting of” or “a composition essentially consisting of”) means that additional molecules may be present but that such molecule does not change/alter the characteristic/activity/functionality of said product or composition. For example, a composition may essentially consist of a VHH according to the invention if the composition as such would exhibit similar characteristic/activity/functionality as said VHH.

The recitation of numerical ranges by endpoints includes all numbers and fractions subsumed within the respective ranges, as well as the recited endpoints.

The term ‘about’ as used herein when referring to a measurable value such as a parameter, an amount, a temporal duration, and the like, is meant to encompass variations of +/−10% or less, preferably +/−5% or less, more preferably +/−1% or less, and still more preferably +/−0.1% or less of and from the specified value, insofar such variations are appropriate to perform in the disclosed invention. It is to be understood that the value to which the modifier ‘about’ refers is itself also specifically, and preferably, disclosed.

The terms “for use in medicine” and “for use as a medicament” are used interchangeably in this application.

### Polypeptide, Nucleic Acid, Identity, Similarity

As used herein, amino acid residues will be indicated either by their full name or according to the standard three-letter or one-letter amino acid code.

As used herein, the terms ‘polypeptide’ or ‘protein’ are used interchangeably, and refer to a polymeric form of amino acids of any length, which can include coded and non-coded amino acids, chemically or biochemically modified or derivatized amino acids, and polypeptides having modified peptide backbones. A “peptide” is also a polymer of amino acids with a length which is usually of up to 50 amino acids. A polypeptide or peptide is represented by an amino acid sequence.

As used herein, the terms ‘nucleic acid molecule’, ‘polynucleotide’, ‘polynucleic acid’, ‘nucleic acid’ are used interchangeably and refer to a polymeric form of nucleotides of any length, either deoxyribonucleotides or ribonucleotides, or analogues thereof. A nucleic acid molecule is represented by a nucleic acid sequence, which is primarily characterized by its base sequence. Polynucleotides may have any three-dimensional structure, and may perform any function, known or unknown. Non-limiting examples of polynucleotides include a gene, a gene fragment, exons, introns, messenger RNA (mRNA), transfer RNA, ribosomal RNA, ribozymes, cDNA, recombinant polynucleotides, branched polynucleotides, plasmids, vectors, isolated DNA of any sequence, control regions, isolated RNA of any sequence, nucleic acid probes, and primers. The nucleic acid molecule may be linear or circular.

As used herein, the term ‘homology’ denotes at least secondary structural identity or similarity between two macromolecules, particularly between two polypeptides or polynucleotides, from same or different taxons, wherein said similarity is due to shared ancestry. Hence, the term ‘homologues’ denotes so-related macromolecules having said secondary and optionally tertiary structural similarity. For comparing two or more nucleotide sequences, the ‘(percentage of) sequence identity’ between a first nucleotide sequence and a second nucleotide sequence may be calculated using methods known by the person skilled in the art, e.g. by dividing the number of nucleotides in the first nucleotide sequence that are identical to the nucleotides at the corresponding positions in the second nucleotide sequence by the total number of nucleotides in the first nucleotide sequence and multiplying by 100% or by using a known computer algorithm for sequence alignment such as NCBI Blast. In determining the degree of sequence similarity between two amino acid sequences, the skilled person may take into account so-called ‘conservative’ amino acid substitutions, which can generally be described as amino acid substitutions in which an amino acid residue is replaced with another amino acid residue of similar chemical structure and which has little or essentially no influence on the function, activity or other biological properties of the polypeptide. Possible conservative amino acid substitutions will be clear to the person skilled in the art. Amino acid sequences and nucleic acid sequences are said to be ‘exactly the same’ if they have 100% sequence identity over their entire length.

Throughout this application, each time one refers to a specific amino acid sequence SEQ ID NO (take SEQ ID NO: Y as example), one may replace it by: a polypeptide comprising an amino acid sequence that has at least 80% sequence identity or similarity with amino acid sequence SEQ ID NO: Y.

Each amino acid sequence described herein by virtue of its identity percentage (at least 80%) with a given amino acid sequence respectively has in a further preferred embodiment an identity of at least 80%, 85%, 90%, 95%, 97%, 98%, 99% or more identity with the given amino acid sequence respectively. In a preferred embodiment, sequence identity is determined by comparing the whole length of the sequences as identified herein. Each amino acid sequence described herein by virtue of its similarity percentage (at least 81%) with a given amino acid sequence respectively has in a further preferred embodiment a similarity of at least 81%, 85%, 90%, 95%, 97%, 98%, 99% or more similarity with the given amino acid sequence respectively. In a preferred embodiment, sequence similarity is determined by comparing the whole length of the sequences as identified herein. Unless otherwise indicated herein, identity or similarity with a given SEQ ID NO means identity or similarity based on the full length of said sequence (i.e. over its whole length or as a whole).

“Sequence identity” is herein defined as a relationship between two or more amino acid (polypeptide or protein) sequences or two or more nucleic acid (polynucleotide) sequences, as determined by comparing the sequences. The identity between two amino acid sequences is preferably defined by assessing their identity within a whole SEQ ID NO as identified herein or part thereof. Part thereof may mean at least 50% of the length of the SEQ ID NO, or at least 60%, or at least 70%, or at least 80%, or at least 90%. In the art, “identity” also means the degree of sequence relatedness between amino acid sequences, as the case may be, as determined by the match between strings of such sequences. “Similarity” between two amino acid sequences is determined by comparing the amino acid sequence and its conserved amino acid substitutes of one polypeptide to the sequence of a second polypeptide. “Identity” and “similarity” can be readily calculated by known methods, including but not limited to those described in Computational Molecular Biology, Lesk, A. M., ed., Oxford University Press, New York, 1988; Biocomputing: Informatics and Genome Projects, Smith, D. W., ed., Academic Press, New York, 1993; Computer Analysis of Sequence Data, Part I, Griffin, A. M., and Griffin, H. G., eds., Humana Press, New Jersey, 1994; Sequence Analysis in Molecular Biology, von Heine, G., Academic Press, 1987; and Sequence Analysis Primer, Gribskov, M. and Devereux, J., eds., M Stockton Press, New York, 1991; and Carillo, H., and Lipman, D., SIAM J. Applied Math., 48:1073 (1988).

Preferred methods to determine identity are designed to give the largest match between the sequences tested. Methods to determine identity and similarity are codified in publicly available computer programs. Preferred computer program methods to determine identity and similarity between two sequences include e.g. the GCG program package (Devereux, J., et al., Nucleic Acids Research 12 (1): 387 (1984)), BestFit, FASTA, BLASTN, and BLASTP (Altschul, S. F. et al., J. Mol. Biol. 215:403-410 (1990)), EMBOSS Needle (Madeira, F., et al., Nucleic Acids Research 47 (W1): W636-W641 (2019)). The BLAST program is publicly available from NCBI and other sources (BLAST Manual, Altschul, S., et al., NCBI NLM NIH Bethesda, MD 20894; Altschul, S., et al., J. Mol. Biol. 215:403-410 (1990)). The EMBOSS program is publicly available from EMBL-EBI. The well-known Smith Waterman algorithm may also be used to determine identity. The EMBOSS Needle program is the preferred program used.

Preferred parameters for polypeptide sequence comparison include the following: Algorithm: Needleman and Wunsch, J. Mol. Biol. 48 (3): 443-453 (1970); Comparison matrix: BLOSUM62 from Henikoff and Henikoff, Proc. Natl. Acad. Sci. USA. 89:10915-10919 (1992); Gap Open Penalty: 10; and Gap Extend Penalty: 0.5. A program useful with these parameters is publicly available as the EMBOSS Needle program from EMBL-EBI. The aforementioned parameters are the default parameters for a Global Pairwise Sequence alignment of proteins (along with no penalty for end gaps).

Preferred parameters for nucleic acid comparison include the following: Algorithm: Needleman and Wunsch, J. Mol. Biol. 48:443-453 (1970); Comparison matrix: DNAfull; Gap Open Penalty: 10; Gap Extend Penalty: 0.5. A program useful with these parameters is publicly available as the EMBOSS Needle program from EMBL-EBI. The aforementioned parameters are the default parameters for a Global Pairwise Sequence alignment of nucleotide sequences (along with no penalty for end gaps).

Optionally, in determining the degree of amino acid similarity, the skilled person may also take into account so-called “conservative” amino acid substitutions, as will be clear to the skilled person. Conservative amino acid substitutions refer to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; a group of amino acids having acidic side chains is aspartate and glutamate; and a group of amino acids having sulphur-containing side chains is cysteine and methionine. Preferred conservative substitutions for each of the naturally occurring amino acids are as follows: Ala to Ser; Arg to Lys or Gln; Asn to Asp, His or Ser; Asp to Glu or Asn; Gln to Glu, Lys or Arg; Glu to Lys, Asp, Gln; His to Tyr or Asn; Ile to Leu, Val, or Met; Leu to Ile, Met or Val; Lys to Arg, Gln or Glu; Met to Val, Leu or Ile; Phe to Trp or Tyr; Ser to Thr, Ala or Asn; Thr to Ser; Trp to Tyr or Phe; Tyr to His, Trp or Phe; and Val to Ile, Leu or Met. Substitutional variants of the amino acid sequence disclosed herein are those in which at least one residue in the disclosed sequences has been removed and a different residue inserted in its place. Preferably, the amino acid change is conservative.

### Antibody

As used herein, the term ‘antibody’ refers to polyclonal antibodies, monoclonal antibodies, humanized antibodies, single-chain antibodies, and fragments thereof such as Fab F(ab′)₂, scFv, VHH and other fragments that retain the antigen binding function of the parent antibody. As such, an antibody may refer to an immunoglobulin or glycoprotein, or fragment or portion thereof, or to a construct comprising an antigen-binding portion comprised within a modified immunoglobulin-like framework, or to an antigen-binding portion comprised within a construct comprising a non-immunoglobulin-like framework or scaffold.

As used herein, the term ‘monoclonal antibody’ refers to an antibody composition having a homogeneous antibody population. The term is not limited regarding the species or source of the antibody, nor is it intended to be limited by the manner in which it is made. The term encompasses whole immunoglobulins as well as fragments and others that retain the antigen binding function of the antibody. Monoclonal antibodies of any mammalian species can be used in this invention. In practice, however, the antibodies will typically be of rat or murine origin because of the availability of rat or murine cell lines for use in making the required hybrid cell lines or hybridomas to produce monoclonal antibodies.

As used herein, the term ‘polyclonal antibody’ refers to an antibody composition having a heterogeneous antibody population. Polyclonal antibodies are often derived from the pooled serum from immunized animals or from selected humans.

‘Heavy chain variable domain of an antibody or a fragment thereof’, as used herein, means (i) the variable domain of the heavy chain of a heavy chain antibody, which is naturally devoid of light chains (also indicated hereafter as VHH), including but not limited to the variable domain of the heavy chain of heavy-chain antibodies of camelids or sharks or (ii) the variable domain of the heavy chain of a conventional four-chain antibody (also indicated hereafter as V_{H}), including but not limited to a camelized (as further defined herein) variable domain of the heavy chain of a conventional four-chain antibody (also indicated hereafter as camelized V_{H}) or any fragments thereof, such as but not limited to one or more stretches of amino acid residues (i.e. small peptides) that are particularly suited for binding to a tumour antigen or an antigen present on cancer cells and which are present in, and/or may be incorporated into, the VHH's as disclosed herein (or may be based on and/or derived from CDR sequences of the VHH's as disclosed herein). In an embodiment, the fragment of a VHH is a functional fragment.

As further described herein below, the amino acid sequence and structure of a heavy chain variable domain of an antibody can be considered, without however being limited thereto, to be comprised of four framework regions or ‘FR's’, which are referred to in the art and herein below as ‘framework region 1’ or ‘FR1’; as ‘framework region 2’ or ‘FR2’; as ‘framework region 3’ or ‘FR3’; and as ‘framework region 4’ or ‘FR4’, respectively, which framework regions are interrupted by three complementary determining regions or ‘CDR's’, which are referred to in the art as ‘complementarity determining region 1’ or ‘CDR1’; as ‘complementarity determining region 2’ or ‘CDR2’; and as ‘complementarity determining region 3’ or ‘CDR3’, respectively.

As used herein, the terms ‘complementarity determining region’ or ‘CDR’ within the context of antibodies refer to variable regions of either the H (heavy) or the L (light) chains (also abbreviated as VH and VL, respectively) and contain the amino acid sequences capable of specifically binding to antigenic targets. These CDR regions account for the basic specificity of the antibody for a particular antigenic determinant structure. Such regions are also referred to as “hypervariable regions.” The CDRs represent non-contiguous stretches of amino acids within the variable regions but, regardless of species, the positional locations of these critical amino acid sequences within the variable heavy and light chain regions have been found to have similar locations within the amino acid sequences of the variable chains. The variable heavy and light chains of all canonical antibodies each have 3 CDR regions, each non-contiguous with the others (termed L1, L2, L3, H1, H2, H3) for the respective light (L) and heavy (H) chains.

As also further described herein below, the total number of amino acid residues in a heavy chain variable domain of an antibody (including a VHH or a V_{H}) can be in the region of 110-130. It should however be noted that parts, fragments or analogs of a heavy chain variable domain of an antibody are not particularly limited as to their length and/or size, as long as such parts, fragments or analogs retain (at least part of) the functional activity, and/or retain (at least part of) the binding specificity of the original heavy chain variable domain of an antibody from which these parts, fragments or analogs are derived from. Parts, fragments or analogs retaining (at least part of) the functional activity, and/or retaining (at least part of) the binding specificity of the original heavy chain variable domain of an antibody from which these parts, fragments or analogs are derived from are also further referred to herein as ‘functional fragments’ of a heavy chain variable domain.

The amino acid residues of a variable domain of an antibody (including a VHH or a V_{H}) are preferably numbered according to the IMGT unique numbering for V-domain (immunoglobulins and T cell receptors) given by the IMGT nomenclature as described (Lefranc M. P. et al 1997 Immunology today, 18:509, PMID: 9386342; Lefranc, M.-P., 1999 The Immunologist, 7:132-136 and Lefranc M. P. et al 2003, Dev. Comp. Immunol., 27:55-77 PMID: 12477501). According to this numbering (see for example table 1 of Lefranc 2003), the conserved amino acids always have the same position, for instance cysteine 23 (1st-CYS), tryptophan 41 (CONSERVED-TRP), hydrophobic amino acid 89, cysteine 104 (2nd-CYS), phenylalanine or tryptophan 118 (J-PHE or J-TRP). The IMGT unique numbering provides a standardized delimitation of the framework regions (FR1-IMGT: positions 1 to 26, FR2-IMGT: 39 to 55, FR3-IMGT: 66 to 104 and FR4-IMGT: 118 to 128) and of the complementarity determining regions: CDR1-IMGT: 27 to 38, CDR2-IMGT: 56 to 65 and CDR3-IMGT: 105 to 117. Gaps represent unoccupied positions. Gaps in the CDR1-IMGT and CDR2-IMGT (less than 12 and 10 amino acid long, respectively) are put at the top of the CDR-IMGT loops.

The basic length of a rearranged CDR3-IMGT is 13 amino acids (positions 105 to 117), which corresponds to a JUNCTION of 15 amino acids (2nd-CYS 104 to J-TRP or J-PHE 118). If the CDR3-IMGT length is less than 13 amino acids, gaps are created from the top of the loop, in the following order 111, 112, 110, 113, 109, 114, etc. If the CDR3-IMGT length is more than 13 amino acids, additional positions are created between positions 111 and 112 at the top of the CDR3-IMGT loop in the following order 112.1, 111.1, 112.2, 111.2, 112.3, 111.3, etc.

In this respect, it should be noted that—as is well known in the art for VHH domains—the total number of amino acid residues in each of the CDR's may vary and may not correspond to the total number of amino acid residues indicated by the IMGT numbering (that is, one or more positions according to the IMGT numbering may not be occupied in the actual sequence, or the actual sequence may contain more amino acid residues than the number allowed for by the IMGT numbering). This means that, generally, the numbering according to IMGT may or may not correspond to the actual numbering of the amino acid residues in the actual sequence.

Alternatively, the amino acid residues of a variable domain of an antibody (including a VHH or a VH) can be numbered according to Kabat numbering (Kabat et al 1987, National Institute of Health; 1987. 804 pp., Publication no. 165-462.). Correspondence between the IMGT and Kabat numbering for the immunoglobulin V-regions can be found for example in Table 2 of Lefranc et al., 2003.

For a general description of heavy chain antibodies and the variable domains thereof, reference is inter alia made to Muyldermans S., et al 2013 Annual Review of Biochemistry, 82:775-797 as general background art.

Generally, it should be noted that the term ‘heavy chain variable domain’ as used herein in its broadest sense is not limited to a specific biological source or to a specific method of preparation. For example, as will be discussed in more detail below, the heavy chain variable domains derived from heavy chain antibodies (i.e. VHH's) according to the invention can be obtained (1) by isolating the VHH domain of a naturally occurring heavy chain antibody; (2) by expression of a nucleotide sequence encoding a naturally occurring VHH domain; (3) by ‘camelization’ (as described below) of a naturally occurring V_{H} domain from any animal species, in particular a species of mammal, such as from a human being, or by expression of a nucleic acid encoding such a camelized V_{H} domain; (4) by ‘camelization’ of a ‘domain antibody’ or ‘dAb’ as described by Weizao C., et al Methods Mol Biol 2009, 525:81-99)), or by expression of a nucleic acid encoding such a camelized V_{H} domain (5) using synthetic or semi-synthetic techniques for preparing proteins, polypeptides or other amino acid sequences; (6) by preparing a nucleic acid encoding a VHH using techniques for nucleic acid synthesis, followed by expression of the nucleic acid thus obtained; and/or (7) by any combination of the foregoing. Suitable methods and techniques for performing the foregoing will be clear to the skilled person based on the disclosure herein and for example include the methods and techniques described in more detail herein below.

An VHH, or a fragment thereof, according to the invention is considered to be ‘(in) essentially isolated (form)’ as used herein, when it has been extracted or purified from the host cell and/or medium in which it is produced.

### Method for Isolating Suitable VHHs Against an Antigen

In particular embodiments, VHHs, or fragments thereof, according to the invention are obtained by affinity selection an antigen present on and/or specific for a solid tumour and/or a cancer cell, preferably human and/or murine FAP, or human FOLR1. Obtaining suitable polypeptides by affinity selection against a particular solid tumour antigen or cancer cell may for example be performed by screening a set, collection or library of cells that express VHHs on their surface (e.g. bacteriophages) for binding against a tumour-specific antigen and/or a cancer cell-specific antigen; all of which may be performed in a manner known per se, essentially comprising the following non-limiting steps: a) obtaining an isolated solution or suspension of a tumour-specific or cancer cell-specific protein target molecule, which molecule is known to be a target for a potential cancer drug; b) bio-panning phages or other cells from a VHH library against said protein target molecule; c) isolating the phages or other cells binding to the tumour-specific or cancer cell-specific protein target molecule; d) determining the nucleotide sequence encoding the VHH insert from individual binding phages or other cells; e) producing an amount of VHH according to this sequence using recombinant protein expression; f) determining the affinity of said VHH domain for said tumour-specific or cancer cell-specific protein target molecule; and optionally g) testing the tumoricidal or anti-cancer activity of said VHH domain in a bio-assay. Various methods may be used to determine the affinity between the VHH domain and the tumour-specific or cancer cell-specific protein target molecule, including for example, enzyme linked immunosorbent assays (ELISA) or Surface Plasmon Resonance (SPR) assays, which are common practice in the art, for example, as described in Sambrook et al. (2001), Molecular Cloning, A Laboratory Manual. Third Edition. Cold Spring Harbor Laboratory Press, Cold Spring Harbor, NY. The equilibrium dissociation constant is commonly used to describe the affinity between a polypeptide and its target molecule. Typically, the equilibrium dissociation constant is lower than 10⁻⁷ M. Preferably, the equilibrium dissociation constant is lower than 10⁻⁸ M, or lower than 10⁻⁹ M, or more preferably, ranged from 10⁻⁹ M to 10⁻¹² M.

### Variant of VHHs

It should be noted that the VHHs, or fragments thereof, according to the invention are not limited as to the origin of the VHH or fragment (or of the nucleotide sequences of the invention used to express them). Furthermore, the present invention is also not limited as to the way that the VHHs, or fragments thereof, have been generated or obtained. Thus, the amino acid sequences as disclosed herein may be synthetic or semi-synthetic amino acid sequences, polypeptides or proteins.

The present invention also encompasses parts, fragments, analogues, mutants, variants, and/or derivatives of the VHHs, or fragments thereof, and/or polypeptides comprising or essentially consisting of one or more of such parts, fragments, analogues, mutants, variants, and/or derivatives, as long as these parts, fragments, analogues, mutants, variants, and/or derivatives are suitable for the purposes envisaged herein, which are preferably: deliver to a CNS molecule, a cell, a tissue or an organ expressing an antigen, preferably FAP or FOLR1, linked to it and suitable to be used in diagnostic and therapeutic applications when linked to a radionuclide. Such parts, fragments, analogues, mutants, variants, and/or derivatives according to the invention: are preferably still capable of specifically binding said antigen, preferably to:
specifically bind to human and/or murine FAP, preferably to both FAP, and/or
are preferably not modulators of FAP, preferably not inhibitors of FAP, and/or
specifically bind to human FOLR1 and not capable of specifically binding to murine FOLR1 and to human FOLR2 and to human FOLR3.

For example, a number of stretches of amino acid residues (i.e. small peptides) are provided herein, also referred to CDR sequences or part of the VHH, or fragment thereof, and identified as SEQ ID NO: 1, 2, 3, such as sequences having at least 80% identity with SEQ ID NO: 4 representing the sequence of the VHH, or fragment thereof, that are particularly suited for binding to human and/or murine FAP.

For example, a number of stretches of amino acid residues (i.e. small peptides) are provided herein, also referred to herein as CDR sequences or part of VHH, or fragment thereof, and identified as SEQ ID NO, such as sequences having at least 60% identity with at least one of SEQ ID NO: 15, 16, 17, 18, 19, 20, 21, 22, 23, 24, 25, 30, 31, 32 of the VHH, or fragment thereof, that are particularly suited for binding to human FOLR1.

These stretches may be regarded as being functional fragments of the VHH and may be present in, and/or may be incorporated into any suitable scaffold (protein), in particular in such a way that they form (part of) the antigen binding site of that suitable scaffold or VHH. It should however be noted that the invention in its broadest sense is not limited to a specific structural role or function that these stretches of amino acid residues may have in the scaffolds or VHHs as disclosed herein, as long as these stretches of amino acid residues allow these scaffolds or VHHs as disclosed herein to specifically bind to said antigen.

### Further Posttranslational Structural Characterization of the VHH or Fragment Thereof

In certain aspects, a VHH, or a fragment thereof, according to the invention may be optionally linked to one or more further groups, moieties, or residues via one or more linkers. These one or more further groups, moieties or residues can serve for binding to other targets of interest. It should be clear that such further groups, residues, moieties and/or binding sites may or may not provide further functionality to the VHH and may or may not modify its properties as disclosed herein. Such groups, residues, moieties or binding units may also for example be chemical groups which can be biologically active.

These groups, moieties or residues are, in particular embodiments, linked N- or C-terminally to the heavy chain variable domain, in particularly C-terminally linked.

In particular embodiments, a VHH, or a fragment thereof, according to the invention may also have been chemically modified. For example, such a modification may involve the introduction or linkage of one or more functional groups, residues or moieties into or onto the VHH. These groups, residues or moieties may confer one or more desired properties or functionalities to the VHH. Examples of such functional groups will be clear to the skilled person.

For example, the introduction or linkage of such functional groups to the VHH can result in an increase in their solubility and/or their stability, in a reduction of their toxicity, or in the elimination or attenuation of any undesirable side effects, and/or in other advantageous properties.

In particular embodiments, one or more groups, residues or moieties are linked to a VHH, or a fragment thereof, according to the invention via one or more suitable linkers or spacers.

In cases where all of the two or more binding sites of a VHH, or a fragment thereof, according to the invention are directed against or specifically bind to the same site, determinant, part, epitope, domain or stretch of amino acid residues of an antigen as disclosed herein is said to be ‘bivalent’ (in the case of two binding sites on the VHH or fragment thereof) or multivalent (in the case of more than two binding sites on the VHH or fragment thereof), such as for example trivalent.

In an embodiment, a VHH, or a fragment thereof, according to the invention is present in a monovalent format.

As used herein, the term ‘monovalent’ when referring to a VHH, or a fragment thereof, according to the invention denotes a VHH, or fragment thereof, in monomeric form. A monovalent VHH or fragment thereof contains only one binding site. In this context, the binding site of a VHH, or a fragment thereof, according to the invention encompasses one or more ‘complementarity determining regions’ or ‘CDRs’ that are directed against or specifically bind to a particular site, determinant, part, epitope, domain or stretch of an antigen.

In embodiments, the binding site of a VHH, or a fragment thereof, according to the invention encompasses one or more ‘complementarity determining regions’ or ‘CDRs’ represented by SEQ ID NO:1, 2 and/or 3 and/or one or more regions identified herein as having at least 80% identity with SEQ ID NO: 4 of a VHH that is directed against or specifically bind to a particular site, determinant, part, epitope, domain or stretch of amino acid residues of human and/or murine FAP.

In embodiments, the binding site of a VHH, or a fragment thereof, according to the invention encompasses the one or more ‘complementarity determining regions’ or ‘CDRs’ and/or the one or more regions identified herein as having at least 60% identity with at least one of SEQ ID NO: 15, 16, 17, 18, 19, 20, 21, 22, 23, 24, 25, 30, 31 of a VHH that is directed against or specifically bind to a particular site, determinant, part, epitope, domain or stretch of amino acid residues of human FOLR1.

A VHH, or a fragment thereof, according to the invention is attractive for therapeutic/diagnostic applications. For specific applications, such a small size may also be attractive if a high tissue penetration is needed in order to reach an optimal therapeutic effect.

Some particularly suitable linkers or spacers include for example, but are not limited to, polypeptide linkers such as glycine linkers, serine linkers, mixed glycine/serine linkers, glycine- and serine-rich linkers or linkers composed of largely polar polypeptide fragments, or homo- or heterobifunctional chemical crosslinking compounds such as glutaraldehyde or, optionally PEG-spaced, maleimides or NHS esters.

For example, a polypeptide linker or spacer may be a suitable amino acid sequence having a length between 1 and 50 amino acids, such as between 1 and 30, and in particular between 1 and 10 amino acid residues. It should be clear that the length, the degree of flexibility and/or other properties of the linker(s) may have some influence on the properties of the VHHs, or fragments thereof, according to the invention, including but not limited to the affinity, specificity or avidity for the tumour target or the target on a cancer cell or pharmacological behaviour. It should be clear that when two or more linkers are used, these linkers may be the same or different.

As used herein, the term ‘untagged’ when referring to a VHH, or a fragment thereof, according to the invention denotes a VHH or fragment thereof that contains no extraneous polypeptide sequences (e.g., contains only said VHH or fragment sequence). Exemplary extraneous polypeptide sequences include carboxy-terminal polypeptide tags, e.g., a His-tag, a cysteine-containing tag (e.g., a GGC-tag as described in Pruszynski et al 2013 Nucl Med Biol 40:52-59), and/or a Myc-tag. A His-tag may contain 4, 5, 6, 7, 8, 9, 10 Histidines. In an embodiment, 6 Histidines are present.

Also in one embodiment, the one or more groups, residues or moieties that may be present do not induce multimerization such as dimerization of a VHH, or a fragment thereof, according to the invention.

Therefore in an embodiment, a VHH, or a fragment thereof, according to the invention is devoid of a tag that induces multimerization such as dimerization, preferably devoid of a cysteine-containing tag, preferably a GGC-tag.

Therefore in an embodiment, a VHH, or a fragment thereof, according to the invention is devoid of a carboxy-terminal polypeptide tag, preferably it is untagged.

Advantageously, kidney retention was shown to be significantly reduced when using a VHH, or a fragment thereof, according to the invention without a carboxy-terminal polypeptide tag compared to a polypeptide tagged, such as His-tagged and Myc-His-tagged antibody fragment (D'Huyvetter et al. (2014), Theranostics. 4 (7): 708-20).

The term ‘bi-specific’ when referring to a VHH, or a fragment thereof, according to the invention implies that either a) two or more of the binding sites of the VHH or fragment are directed against or specifically bind an antigen but not to the same (i.e. to a different) site, determinant, part, epitope, domain or stretch of amino acid residues of the antigen, a VHH, or a fragment thereof, according to the invention is said to be ‘bi-specific’ (in the case of two binding sites on the VHH or fragment) or multispecific (in the case of more than two binding sites on VHH or fragment) or b) two or more binding sites of the VHH or fragment are directed against or specifically bind to different target molecules of interest. The term ‘multispecific’ is used in the case that more than two binding sites are present on the VHH or fragment.

The ‘half-life’ of a VHH, or a fragment thereof, according to the invention can generally be defined as the time that is needed for the in vivo serum concentration of the VHH or fragment to be reduced by 50%. The in vivo half-life of a VHH, or a fragment thereof, according to the invention can be determined in any manner known to the person skilled in the art, such as by pharmacokinetic analysis. As will be clear to the skilled person, the half-life can be expressed using parameters such as the t1/2-alpha, t1/2-beta and the area under the curve (AUC). An increased half-life in vivo is generally characterized by an increase in one or more and preferably in all three of the parameters t1/2-alpha, t1/2-beta and the area under the curve (AUC).

The term “lifetime extended” when referring to a VHH, or a fragment thereof, according to the invention is used to denote that the VHH or fragment has been modified to extend the half-life of the VHH or fragment. Strategies for extending the half-life of VHHs are well-known in the art and include for example, but without limitation, linkage (chemically or otherwise) to one or more groups or moieties that extend the half-life, such as polyethylene glycol (PEG) or bovine serum albumin (BSA) or human serum albumin (HSA), antibody Fc fragments, or antigen-binding antibody fragments targeting serum proteins such as serum albumin. Therefore, in an embodiment, a VHH, or a fragment thereof, according to the invention is non-lifetime extended.

### Binding

Specific binding of a VHH, or a fragment thereof, according to the invention can be determined in any suitable manner known per se, including for example biopanning, Scatchard analysis and/or competitive binding assays, such as radioimmunoassays (RIA), enzyme immunoassays (EIA) (also called Enzyme-Linked Immuno Sorbent Assay, ELISA), sandwich competition assays, Surface Plasmon Resonance (SPR), or Bio-Layer Interferometry and the different variants thereof known in the art. Each of these assays may be carried out in vitro using an antigen, preferably a human and/or murine FAP recombinant protein or a human FOLR1 recombinant protein or a human HER2 recombinant protein, which may be immobilised on a support or in solution. Alternatively, under some specific circumstances, some of these assays may be carried out in vitro using cells that express the corresponding antigen, preferably human and/or murine FAP, or human FOLR1, or human HER2. Such cells may endogenously express or overexpress express the antigen, preferably human and/or murine FAP, or human FOLR1, or human HER2. The assessment is usually carried out in vitro in a culture medium or in PBS or in a suitable medium or buffer.

A preferred cell is a fibroblast cell expressing human and/or murine FAP. A preferred cell line may be GM05389 or U-87 MG. Alternatively a preferred cell is a transfected cell expressing human and/or murine FAP. A preferred transfected cell line may be HEK293.

A preferred cell is SKOV3 or OVCAR3 expressing human FOLR1.

Alternatively, the binding of a VHH, or a fragment thereof, according to the invention may be assessed in vivo in an animal expressing the corresponding antigen, preferably human and/or murine FAP or human FOLR1 or human HER2, preferably using an imaging technique is used. A preferred imaging technique is SPECT/CT, PET/CT, SPECT/MRI or PET/MRI. Cells overexpressing an antigen, preferably human and/or murine FAP or human FOLR1 or human HER2, may also be xenografted into the animal. It is also possible to use the human FAP or FOLR1 or HER2 knock-in mouse of the invention expressing human FAP or FOLR1 or HER2.

The wording “in vitro” is therefore used herein in the context of a cell-free assay when the human and/or murine FAP or human FOLR or human HER2 recombinant protein is immobilized on a support or in solution, or in the context of a cell in culture. As opposed to that, the wording “in vivo” or “ex vivo” is used herein in the context of a non-human animal or a tissue or organ of this non-human animal. Usually “ex vivo” is used when a quantification is carried out on a tissue or organ of a non-human or human animal and “in vivo” is used when a quantification via an imaging method is carried out on a non-human or human animal. Usually “binding” is assessed using in vitro conditions and is further confirmed using in vivo conditions.

In the in vitro, in vivo and ex vivo assays disclosed herein, it is preferred to use a negative control. It is also possible to assess the specific binding to an antigen, preferably to human and/or murine FAP or human FOLR1 or human HER2, in the presence of other antigens as explained later herein.

The term “affinity”, “specific binding”, “binding”, “binding activity” or “specific binding activity”, as used herein, refers to the degree to which a VHH, or a fragment thereof, according to the invention binds to an antigen, preferably human and/or murine FAP or human FOLR1 or human HER2, so as to shift the equilibrium of the VHH or fragment and the antigen towards the presence of a complex formed by their binding. The binding may be assessed using SPR or bio-layer interferometry. Thus, for example, where human and/or murine FAP and the VHH or fragment are combined in relatively equal concentrations, the VHH or fragment of high affinity will bind to the available human and/or murine FAP so as to shift the equilibrium towards high concentrations of the resulting complex. The equilibrium dissociation constant (K_{D}) is commonly used to describe the affinity between the protein binding domain (VHH or fragment) and the antigen.

Typically, the equilibrium dissociation constant is less than 10⁻⁷ M. Preferably, the equilibrium dissociation constant is less than 10⁻⁸ M, or less than 10⁻⁹M, or more preferably, ranging from 10⁻⁹ M and 10⁻¹² M. Any VHH or fragment thereof as disclosed herein is preferably such that it specifically binds (as defined herein) to human and/or murine FAP or human FOLR1 or human HER2 with an equilibrium dissociation constant (K_{D}) ranging from 10⁻⁹ to 10⁻¹² moles/liter or from 10⁻¹⁰ to 10⁻¹² moles/liter, preferably assessed using bio-layer interferometry.

The ‘specificity’ of a VHH, or a fragment thereof, according to the invention can be determined based on affinity and/or avidity. The ‘affinity’ of a VHH, or a fragment thereof, according to the invention is represented by the equilibrium constant for the dissociation of the VHH or fragment and the antigen to which it binds.

The lower the K_{D} value, the stronger the binding strength between the VHH or fragment and the target protein (antigen) of interest to which it binds. Alternatively, the affinity can also be expressed in terms of the equilibrium association constant (K_{A}), which corresponds to 1/K_{D}. The binding affinity of a VHH, or a fragment thereof, according to the invention can be determined in a manner known to the skilled person, depending on the specific target protein of interest. The ‘avidity’ of a VHH, or a fragment thereof, according to the invention is the measure of the strength of binding between the VHH or fragment and the pertinent target protein of interest. Avidity is related to both the affinity between a binding site on the target protein of interest and a binding site on the VHH or fragment and the number of pertinent binding sites present on the VHH or fragment. VHHs have only one single-domain and therefore only one single binding site. The affinity exhibited by VHHs is in the sub-nanomolar range and is therefore quite exceptional in view of the presence of a single binding site. A K_{D} value greater than about 1 millimolar is generally considered to indicate non-binding or non-specific binding. It is generally known in the art that the K_{D} can also be expressed as the ratio of the dissociation rate constant of a complex, denoted as K_{off} or k_{d} (expressed in seconds⁻¹ or s⁻¹), to the rate constant of its association, denoted kₒₙ or k_{d} (expressed in molar⁻¹ seconds⁻¹ or M⁻¹ s⁻¹). In particular, a VHH, or a fragment thereof, according to the invention may bind to the target protein of interest (i.e. antigen) with a k_{off} ranging from 0.1 and 0.00001 s⁻¹, or ranging from 10⁻² to 10⁻⁵ s⁻¹, or ranging from 10⁻³ to 10⁻⁵ s⁻¹, or ranging from 10⁻⁴ to 10⁻⁵ s⁻¹ and/or a kₒₙ ranging from 1,000 and 10,000,000 M⁻¹ s⁻¹ or ranging from 10⁻⁴ to 10⁻⁷ M⁻¹ s⁻¹ or from 10⁻⁵ to 10⁻⁷ M⁻¹s⁻¹. Binding affinities, K_{off} and Kₒₙ rates may be determined by means of methods known to the person skilled in the art, for example ELISA methods, isothermal titration calorimetry, SPR, bio-layer interferometry, fluorescence-activated cell sorting analysis, and the more. In a preferred embodiment, the antibody fragment as disclosed herein specifically binds to human and/or murine FAP with a K_{off} ranging from 0.1 and 0.00001s⁻¹, or ranging from 10⁻² to 10⁻⁵ s⁻¹ or from 10⁻³ to 10⁻⁵ s⁻¹, or ranging from 10⁻⁴ to 10⁻⁵ s⁻¹ preferably assessed using bio-layer interferometry.

In a preferred embodiment, a VHH, or a fragment thereof, according to the invention as disclosed herein is such that it specifically binds (as defined herein) to human and/or murine FAP with a K_{D} ranged from 10⁻⁹ to 10⁻¹² moles/liter and/or a K_{off} ranging from 10⁻² to 10⁻⁵ s⁻¹ preferably assessed using bio-layer interferometry, more preferably with a K_{D} ranged from 10⁻⁹ to 10⁻¹² moles/liter and a k_{off} ranging from 10⁻² to 10⁻⁵ s⁻¹.

Accordingly, a VHH, or a fragment thereof, according to the invention is said to ‘specifically bind to’ an antigen, preferably human and/or murine FAP or human FOLR1 or human HER2, when that VHH or fragment thereof has affinity for, specificity for and/or is specifically directed against that antigen (or for at least one part or fragment thereof).

In respect of a VHH, or a fragment thereof, according to the invention, the terms ‘binding region’, ‘binding site’ or ‘interaction site’ present on the VHH or fragment shall herein have the meaning of a particular site, part, locus, domain or stretch of amino acid residues present on the VHH or fragment that is responsible for binding or specific binding to the corresponding antigen, preferably human and/or murine FAP or human FOLR1 or human HER2. This binding region present on the VHH or fragment is called a paratope. Such binding region comprises, consists or essentially consists of specific amino acid residues from the amino acid sequence as disclosed herein of the VHH or fragment which are in contact with the corresponding antigen, preferably with human and/or murine FAP or human FOLR1 or human HER2. The region or part or discrete amino acids of the extracellular domain of the antigen, preferably human and/or murine FAP or human FOLR1 or human HER2, that is in contact with said VHH or fragment may be called an epitope and are defined later herein.

The terms ‘specifically bind’ and ‘specific binding’, as used herein, generally refers to the ability of a polypeptide, in particular an immunoglobulin, such as an antibody, or a VHH, or a fragment thereof, according to the invention, such as a VHH, or a fragment thereof, according to the invention, to preferentially bind to a particular antigen such as human and/or murine FAP or human FOLR1 or human HER2. Such VHH, or a fragment thereof, according to the invention may also be identified as VHH, or a fragment thereof, according to the invention raised against that particular antigen.

Methods of producing and manufacturing VHHs

A VHH, or a fragment thereof, according to the invention may be produced or manufactured by any of the methods describes below.

As will be clear to the skilled person, one particularly useful method for preparing a VHH, or a fragment thereof, according to the invention generally comprises the steps of:
(a) expressing a nucleotide sequence encoding the VHH or fragment and
(b) optionally isolating and/or purifying the VHH or fragment.

The nucleic acid encoding VHH or fragment may be comprised in a vector or genetic construct. In particular methods, the VHH or fragment can be obtained by methods which involve generating a random library of VHH sequences and screening this library for a VHH sequence capable of specifically binding to a corresponding antigen, preferably human and/or murine FAP or human FOLR1 or human HER2.

Accordingly, particular methods for preparing a VHH, or a fragment thereof, according to the invention comprise the steps of
a) providing a set, collection or library of amino acid sequences of VHH domains; and
b) screening said set, collection or library of amino acid sequences for amino acid sequences that can bind to and/or have affinity for a corresponding antigen, preferably human and/or murine FAP or human FOLR1 or human HER2.
and
c) isolating the amino acid sequence(s) that can bind to and/or have affinity for the antigen.

In such a method, the set, collection or library of VHH sequences may be any suitable set, collection or library of amino acid sequences. For example, the set, collection or library of amino acid sequences may be a set, collection or library of immunoglobulin fragment sequences (as described herein), such as a naïve set, collection or library of immunoglobulin fragment sequences; a synthetic or semi-synthetic set, collection or library of immunoglobulin fragment sequences; and/or a set, collection or library of immunoglobulin fragment sequences that have been subjected to affinity maturation.

In particular embodiments of this method, the set, collection or library of VHH sequences may be an immune set, collection or library of immunoglobulin fragment sequences, for example derived from a mammal that has been suitably immunized with a corresponding antigen, preferably human and/or murine FAP or human FOLR1 or human HER2 or with a suitable antigenic determinant based thereon or derived therefrom, such as an antigenic part, fragment, region, domain, loop or other epitope thereof. In one particular aspect, said antigenic determinant may be an extracellular part, region, domain, loop or other extracellular epitope(s).

In the above methods, the set, collection or library of VHH sequences may be displayed on a phage, phagemid, ribosome or suitable micro-organism (such as yeast), such as to facilitate screening. Suitable methods, techniques and host organisms for displaying and screening (a set, collection or library of) amino acid sequences will be clear to the person skilled in the art, for example on the basis of the further disclosure herein. Reference is also made to the review by Hoogenboom in Nature Biotechnology, 23, 9, 1105-1116 (2005).

Particular methods for generating a VHH, or a fragment thereof, according to the invention comprise at least the steps of:
a) providing a collection or sample of cells expressing VHH domain amino acid sequences;
b) screening said collection or sample of cells for cells that express an amino acid sequence that can bind to and/or have affinity for a corresponding antigen, preferably human and/or murine FAP or human FOLR1 or human HER2;
and
c) either (i) isolating said amino acid sequence; or (ii) isolating from said cell a nucleic acid sequence that encodes said amino acid sequence, followed by expressing said amino acid sequence.

The collection or sample of cells may for example be a collection or sample of B-cells. Also, in this method, the sample of cells may be derived from a mammal that has been suitably immunized with a corresponding antigen, preferably human and/or murine FAP or human FOLR1 or human HER2, or with a suitable antigenic determinant based thereon or derived therefrom, such as an antigenic part, fragment, region, domain, loop or other epitope thereof. In one particular embodiment, the antigenic determinant may be an extracellular part, region, domain, loop or other extracellular epitope(s).

Particular methods for generating a VHH, or a fragment thereof, according to the invention may comprise at least the steps of:
a) providing a set, collection or library of nucleic acid sequences encoding a VHH domain amino acid sequence;
b) screening said set, collection or library of nucleic acid sequences for nucleic acid sequences that encode an amino acid sequence that can bind to and/or has affinity for a corresponding antigen, preferably human and/or murine FAP or human FOLR1 or human HER2;
and
c) isolating said nucleic acid sequence, followed by expressing said amino acid sequence.

In the above methods, the set, collection or library of nucleic acid sequences encoding amino acid sequences may for example be a set, collection or library of nucleic acid sequences encoding a naïve set, collection or library of immunoglobulin fragment sequences; a set, collection or library of nucleic acid sequences encoding a synthetic or semi-synthetic set, collection or library of immunoglobulin fragment sequences; and/or a set, collection or library of nucleic acid sequences encoding a set, collection or library of immunoglobulin fragment sequences that have been subjected to affinity maturation.

In particular, in such a method, the set, collection or library of nucleic acid sequences encodes a set, collection or library of a VHH, or a fragment thereof, according to the invention directed against an antigen, preferably human and/or murine FAP or human FOLR1 or human HER2.

In the above methods, the set, collection or library of nucleotide sequences may be displayed on a phage, phagemid, ribosome or suitable micro-organism (such as yeast), such as to facilitate screening. Suitable methods, techniques and host organisms for displaying and screening (a set, collection or library of) nucleotide sequences encoding amino acid sequences will be clear to the person skilled in the art, for example on the basis of the further disclosure herein. Reference is also made to the review by Hoogenboom in Nature Biotechnology, 23, 9, 1105-1116 (2005).

The invention also relates to a VHH, or a fragment thereof, according to the invention that are obtainable or obtained by the above methods, or alternatively by a method that comprises one of the above methods and in addition at least the steps of determining the nucleotide sequence or amino acid sequence of said VHH or fragment; and of expressing or synthesizing said VHH or fragment in a manner known per se, such as by expression in a suitable host cell or host organism or by chemical synthesis.

In some cases, the methods for producing a VHH, or a fragment thereof, according to the invention binding specifically to an antigen, preferably to human and/or murine FAP or human FOLR1 or human HER2, may further comprise the step of isolating from the amino acid sequence library at least one VHH or fragment thereof having detectable binding affinity for, or detectable in vitro effect on the antigen.

These methods may further comprise the step of amplifying a sequence encoding at least one VHH or fragment thereof having detectable binding affinity for, or detectable in vitro effect on the activity of the antigen, preferably of human and/or murine FAP or human FOLR1 or human HER2. For example, a phage clone displaying a particular amino acid sequence, obtained from a selection step of a method described herein, may be amplified by reinfection of a host bacteria and incubation in a growth medium.

Particular methods may encompass determining the sequence of the one or more amino acid sequences capable of binding to the antigen, preferably human and/or murine FAP or human FOLR1 or human HER2.

Where a VHH, or a fragment thereof, according to the invention, comprised in a set, collection or library of amino acid sequences, is displayed on a suitable cell or phage or particle, it is possible to isolate from said cell or phage or particle, the nucleotide sequence that encodes that amino acid sequence. In this way, the nucleotide sequence of the selected amino acid sequence library member(s) can be determined by a routine sequencing method.

Particular methods for producing a VHH, or a fragment thereof, according to the invention comprise the step of expressing said nucleotide sequence(s) in a host organism under suitable conditions, so as to obtain the actual desired amino acid sequence. This step can be performed by methods known to the person skilled in the art.

In addition, the obtained VHH or fragment having detectable binding affinity for, and/or no detectable in vitro effect on an activity of the antigen, preferably of human and/or murine FAP or human FOLR1 or human HER2, may be synthesized as soluble protein construct, optionally after their sequence has been identified.

For instance, a VHH, or a fragment thereof, according to the invention obtained, obtainable or selected by the above methods can be synthesized using recombinant or chemical synthesis methods known in the art. Also, the amino acid sequences obtained, obtainable or selected by the above methods can be produced by genetic engineering techniques. Thus, methods for synthesizing the VHH or fragment obtained, obtainable or selected by the above methods may comprise transforming or infecting a host cell with a nucleic acid or a vector encoding an amino acid sequence having detectable binding affinity for, and/or no detectable in vitro effect on an activity of the antigen, preferably human and/or murine FAP or human FOLR1 or human HER2. Accordingly, the VHH or fragment having detectable binding affinity for, and/or no detectable in vitro effect on an activity of the antigen can be made by recombinant DNA methods. DNA encoding the amino acid sequences can be readily synthesized using conventional procedures. Once prepared, the DNA can be introduced into expression vectors, which can then be transformed or transfected into host cells such as E. coli or any suitable expression system, in order to obtain the expression of amino acid sequences in the recombinant host cells and/or in the medium in which these recombinant host cells reside.

It should be understood, as known by someone skilled in the art of protein expression and purification, that a VHH, or a fragment thereof, according to the invention produced from an expression vector using a suitable expression system may be tagged (typically at the N-terminal or C-terminal end of the amino acid sequence) with e.g. a His-tag or other sequence tag for easy purification.

Transformation or transfection of nucleic acids or vectors into host cells may be accomplished by a variety of means known to the person skilled in the art including calcium phosphate-DNA co-precipitation, DEAE-dextran-mediated transfection, polybrene-mediated transfection, electroporation, microinjection, liposome fusion, lipofection, protoplast fusion, retroviral infection, and biolistics.

Suitable host cells for the expression of the desired heavy chain variable domain sequences may be any eukaryotic or prokaryotic cell (e.g., bacterial cells such as E. coli, yeast cells, mammalian cells, avian cells, amphibian cells, plant cells, fish cells, and insect cells), whether located in vitro or in vivo. For example, host cells may be located in a transgenic plant.

Thus, the application also provides methods for the production of a VHH, or a fragment thereof, according to the invention having detectable binding affinity for, or detectable in vitro effect on the activity of an antigen, preferably human and/or murine FAP or human FOLR1 or human HER2, comprising transforming, transfecting or infecting a host cell with nucleic acid sequences or vectors encoding such VHH or fragment and expressing their amino acid sequences under suitable conditions.

This application further provides methods for the manufacture (‘or the production of’ which is equivalent wording) a composition according to the invention.

Particularly, this application provides methods for producing a composition according to the invention, at least comprising the steps of:
obtaining at least one VHH or fragment thereof according to the invention, which specifically binds to an antigen, preferably human and/or murine FAP or human FOLR1 or human HER2, and
formulating said VHH or fragment in a pharmaceutical composition.

In particular embodiments of these methods, the step of obtaining at least one VHH or fragment comprises:
(a) expressing a nucleotide sequence encoding a VHH or fragment thereof according to the invention, which specifically binds to an antigen, preferably human and/or murine FAP or human FOLR1 or human HER2, and optionally
(b) isolating and/or purifying the VHH or fragment.

In other particular embodiments of these methods, the step of obtaining at least one VHH or fragment, which specifically binds to an antigen, preferably human and/or murine FAP of human FOLR1 or human HER2, comprises:
a) providing a set, collection or library of VHH domain sequences or fragments of VHH sequences;
b) screening said set, collection or library of VHH domain sequences or sequences of fragments thereof for sequences that specifically bind to and/or have affinity for the antigen, preferably human and/or murine FAP or human FOLR1 or human HER2, and optionally
c) isolating the VHH sequences or sequences of fragments thereof that specifically bind to and/or have affinity for the antigen, preferably human and/or murine FAP or human FOLR1 or human HER2.

### Formulation/Uses in Therapy/Diagnosis

In the context of the therapeutic or diagnostic use of a combination according to the invention, the dose is defined as the amount of labelled compound or label administered. As such, the administration of given dose of a label or a labelled compound, either for diagnostic or therapeutic purposes or uses, refers to the use of a combination according to the invention, wherein the amount of the labelled compound during the subsequent administration is said dose.

The skilled person knows how to determine an appropriate dosing and application schedule, both of the first and subsequent administration in the context of a combination according to the invention, depending on the nature of the disease and the constitution of the patient. In particular, the skilled person knows how to assess dose-limiting toxicity (DLT) and how to determine the maximum tolerated dose (MTD) accordingly.

In particular embodiments the use of a combination according to the invention comprises the subsequent administration of a labelled compound at a radioactive dosage of lower than about 800 mCi, such as for instance lower than about 150 mCi, such as for instance lower than about 30 mCi, such as lower than about 15 mCi.

In particular embodiments the use of a combination according to the invention comprises the subsequent administration of a labelled compound with a specific activity from about 0.5 mCi/mg to about 8000 mCi/mg, such as for instance from 1 mCi/mg to about 1500 mCi/mg, such as for instance from 1 mCi/mg to about 300 mCi/mg, such as for instance from 1 mCi/mg to about 150 mCi/mg, depending on the radionuclide. The first and/or subsequent administration may be intravenous, intraperitoneal or via another route such as intrathecal. Depending on the desired duration and effectiveness of the treatment, the combination may be administered once or several times, in combination with other therapeutic drugs or radio-sensitizing agents.

The amount of the labelled compounds applied depends on the precise nature of the carcinoma. The dose of radioactivity per administration must be high enough to be effective, but must be below the dose limiting toxicity (DLT).

As used herein, a “screening dose” or a “biomarker dose” is a dose of an agent, such as a labelled compound as described herein, that is sufficient for selecting a subject for treatment, such as a dose that can bind to a cancer cell or solid tumour in the subject and subsequently be detected at the location of the cancer cell or solid tumour, e.g., by imaging the subject using gamma camera imaging such as planar gamma camera imaging, single photon emission computed tomography or positron emission tomography, optionally combined with a non-nuclear imaging technique such as X-ray imaging, computed tomography and/or magnetic resonance imaging. In some embodiments, a screening dose is a dose that is not therapeutically effective. In some embodiments, the screening dose is different than (e.g., lower than) a therapeutic dose as described herein.

As used herein, a “therapeutic dose” is a dose of an agent, such as a labelled compound as described herein, that is therapeutically effective in at least 1%, at least 5%, at least 10%, at least 20%, at least 30%, at least 40%, or at least 50% of subjects in need of such treatment (e.g., in subjects having cancer). In some embodiments, the therapeutic dose is higher than a screening dose as described herein.

As used herein, “imaging a subject” refers to capturing one or more images of a subject using a device that is capable of detecting a label as described herein. The one or more images may be further altered by a computer program and/or a person skilled in the art in order to enhance the images (e.g. by adjusting contrast or brightness of the one or more images). Any device capable of detecting label as described herein is contemplated for use, such as a device for gamma camera imaging such as planar gamma camera imaging, for single photon emission computed tomography or for positron emission tomography, or a device able to combine a nuclear imaging technique with an anatomical imaging technique such as X-ray imaging, computed tomography and/or magnetic resonance imaging. For example, such device can be a device for single photon emission computed tomography/computed tomography (SPECT/CT) or positron emission computed tomography/computed tomography (PET/CT) imaging. Such devices are known in the art and commercially available.

In some embodiments, the administration of the screening dose and the detection by imaging are separated by at least 1 about minute, at least 5 about minutes, at least about 10 minutes, at least about 20 minutes, at least about 30 minutes, at least about 40 minutes, at least about 50 minutes, at least about 1 hour, at least about 1.5 hours, at least about 2 hours, at least about 3 hours, at least about 4 hours, at least about 5 hours, at least about 6 hours, at least about 7 hours, at least about 8 hours, at least about 9 hours, at least about 10 hours, at least about 11 hours, at least about 12 hours, at least about 24 hours, at least about 2 days, or at least about 7 days. In some embodiments, the administration of the screening dose and the detection are separated by between about 1 hour and about 24 hours.

In some embodiments, the screening dose and the therapeutic dose are administered at least about 1 day, at least about 2 days, at least about 3 days, at least about 4 days, at least about 5 days, at least about 6 days, at least about 1 week, at least about 2 weeks, at least about 3 weeks, at least one month, at least about 2 months, or at least about 6 months apart. In some embodiments, the screening dose and the therapeutic dose are administered between about 1 day and about 6 months apart (e.g., between about 1 day and about 2 months, between about 1 day and about 1 month, or between about 1 day and about 1 week apart).

The screening dose and therapeutic dose may each independently be administered by any suitable route, such as systemically, locally or topically. Exemplary routes include intravenous, intraperitoneal, and intrathecal administration. The particular route utilized may, in some embodiments, depend on the nature of the disease (e.g., type, grade, location and stage of the tumour or cancer cell etc.) and the type of subject (e.g., species, constitution, age, gender, weight, etc.).

As used herein for all diagnostic and therapeutic applications, the term “subject” generally refers to a mammal, such as a human, a non-human primate, a rat, a mouse, a rabbit, a dog, a cat, a pig, a horse, a goat, or a sheep. In some embodiments, the subject is a human subject. In some embodiments, the subject is a subject having cancer (e.g., a human subject having cancer). Methods for identifying subjects having cancer include detection of tumour antigens or other tumour biomarkers, genetic testing, MRI, X-ray, PET or SPECT scan, biopsies, and combinations thereof.

As used herein, the terms ‘diagnosis’, ‘prediction’ and/or ‘prognosis’ as used herein comprise diagnosing, predicting and/or prognosing a certain disease and/or disorder and/or condition, thereby predicting the onset and/or presence of a certain disease and/or disorder and/or condition, and/or predicting the progress and/or duration of a certain disease and/or disorder and/or condition, and/or predicting the response of a patient suffering from of a certain disease and/or disorder and/or condition to therapy.

In some embodiments, a screening dose (i.e. used in a diagnostic method) is a dose that is not therapeutically effective. In some embodiments, the screening dose is lower than a therapeutic dose as described herein (e.g., at least about 10 times, at least about 20 times, at least about 30 times, at least about 40 times, at least about 50 times, at least about 100 times, at least about 200 times, at least about 300 times, at least about 400 times, at least about 500 times or at least about 1000 times lower than a therapeutic dose as described herein, or at least about 10, at least about 20, at least about 30, at least about 40, at least about 50, at least about 60, at least about 70, at least about 80, at least about 90, at least about 100, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 320, at least about 340, at least about 360, at least about 380, at least about 400, at least about 450, at least about 500, at least about 1000, at least about 5000, at least about 10000, at least about 13000, at least about 15000, at least about 18000, or at least about 20000 MBq lower than a therapeutic dose as described herein). In some embodiments, the screening dose is between 10 about MBq and about 400 MBq, between about 20 MBq and about 400 MBq, between about 30 MBq and about 400 MBq, about 40 MBq and about 400 MBq, between about 50 MBq and about 400 MBq, between about 100 MBq and about 400 MBq, between about 200 MBq and about 400 MBq, between about 300 MBq and about 400 MBq, between about 10 MBq and about 300 MBq, between about 20 MBq and about 300 MBq, between about 30 MBq and about 300 MBq, about 40 MBq and about 300 MBq, between about 50 MBq and about 300 MBq, between about 100 MBq and about 300 MBq, or between about 200 MBq and about 300 MBq. In some embodiments, the screening dose is between about 7 MBq and about 370 MBq. It is to be understood that any screening dose described herein may be combined with any therapeutic dose as described herein.

In some embodiments, the therapeutic dose is higher than a screening dose as described herein (e.g., at least about 10 times, at least about 20 times, at least about 30 times, at least about 40 times, at least about 50 times, at least about 100 times, at least about 200 times, at least about 300 times, at least about 400 times, at least about 500 times or at least about 1000 times higher than a screening dose as described herein, or at least about 10, at least about 20, at least about 30, at least about 40, at least about 50, at least about 60, at least about 70, at least about 80, at least about 90, at least about 100, at least about 120, at least about 130, at least about 140, at least about 150, at least about 160, at least about 170, at least about 180, at least about 190, at least about 200, at least about 210, at least about 220, at least about 230, at least about 240, at least about 250, at least about 260, at least about 270, at least about 280, at least about 290, at least about 300, at least about 320, at least about 340, at least about 360, at least about 380, at least about 400, at least about 450, at least about 500, at least about 1000, at least about 5000, at least about 10000, at least about 13000, at least about 15000, at least about 18000, or at least about 20000 MBq higher than a screening dose as described herein). In some embodiments, the therapeutic dose is between about 300 MBq and about 20000 MBq, between about 400 MBq and about 20000 MBq, between about 500 MBq and about 20000 MBq, between about 1000 MBq and about 20000 MBq, between about 2000 MBq and about 20000 MBq, between about 3000 MBq and about 20000 MBq, between about 4000 MBq and about 20000 MBq, between about 5000 MBq and about 20000 MBq, between about 10000 MBq and about 20000 MBq, between about 300 MBq and about 10000 MBq, between about 400 MBq and about 10000 MBq, between about 500 MBq and about 10000 MBq, between about 1000 MBq and about 10000 MBq, between about 2000 MBq and about 10000 MBq, between about 3000 MBq and about 10000 MBq, between about 4000 MBq and about 10000 MBq, or between about 5000 MBq and about 10000 MBq. In some embodiments of any one of the methods provided, the therapeutic dose is between about 370 MBq and about 18500 MBq.

The screening and/or therapeutic dose may conveniently be presented in a single dose or as divided doses (which can again be sub-dosed) administered at appropriate intervals. An administration regimen of the therapeutic dose could include long-term (e.g., at least two weeks, and for example several months or years) or daily treatment. In some embodiments, an administration regimen of the therapeutic dose can vary between once a day to once a month, such as between once a day and once every two weeks, such as but not limited to once a week. Thus, in some embodiments, combinations as disclosed herein may be administered once or several times, also intermittently, for instance on a daily basis for several days, weeks or months.

The particular screening dose and therapeutic dose utilized may, in some embodiments, depend on the nature of the disease (such as cancer but also the type, grade, and stage of the tumour or cancer cell etc., or such as any of the other diseases identified herein) and the type of subject (e.g., species, constitution, age, gender, weight, etc.).

In some aspects, the invention provides kits. In some embodiments, the kit comprises a screening dose of a labelled compound as described herein and a therapeutic dose of the same compound. Screening doses and therapeutic doses are described herein.

In some embodiments of any one of the kits, the kit further comprises one or more means for injection of the screening dose and the therapeutic dose. In some embodiments, the screening dose and therapeutic dose are each individually housed in a means for injection. In some embodiments, the means for injection is a syringe. In some embodiments of any one of the kits, the kit further comprises instructions for carrying out a method as described herein (e.g., a method of stratifying and treating a subject as described herein). The instructions may be in any suitable form, e.g., in printed form (e.g., as a paper or laminated insert or label) or in electronic form (e.g., on a disc or USB stick).

Dose, route of administration, application scheme, repetition and duration of treatment will in general depend on the nature of the disease (type, grade, and stage of the tumour or cancer cell or type, grade and stage of the disease or condition further defined herein) and the patient (constitution, age, gender etc.), and will be determined by the skilled medical expert responsible for the treatment. With respect to the possible doses for the components of the disclosed combination which are described above, it is clear that the medical expert responsible for the treatment will carefully monitor whether any dose-limiting toxicity or other severe side effects occur and undertake the necessary steps to manage those.

Generally, for pharmaceutical (diagnostic and therapeutic) use, the components of a combination according to the formulation may be formulated as pharmaceutical preparations or compositions comprising at least one pharmaceutically acceptable carrier, diluent or excipient and/or adjuvant, and optionally one or more further pharmaceutically active polypeptides and/or compounds. Such compositions may be suitable for intraperitoneal, intravenous or other administration such as intrathecal administration. Thus, the first and subsequent administration may be systemically, locally or topically to the tissue or organ of interest, depending on the location, type and origin of the tumour or cancer cell, and preferably intraperitoneally, intravenously or intrathecally, depending on the specific pharmaceutical formulations or compositions to be used. The clinician will be able to select a suitable route of administration and a suitable pharmaceutical formulation or composition to be used in such administration.

The pharmaceutical dosage forms suitable for injection or infusion can include sterile aqueous solutions or dispersions or sterile powders comprising the active ingredients which are adapted for the extemporaneous preparation of sterile injectable or infusible solutions or dispersions, optionally encapsulated in liposomes.

In all cases, the ultimate dosage form must be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof.

The amount of the labelled compound as envisaged herein required for use in prophylaxis and/or treatment may vary with the route of administration, the nature of the condition being treated and the age and condition of the patient and will be ultimately at the discretion of the attendant physician or clinician. Also, the dosage of the labelled compound envisaged herein may vary depending on the target cell, tumour, tissue, graft, or organ.

In particular, the labelled compound as envisaged herein will be administered in an amount which will be determined by the medical practitioner based inter alia on the severity of the condition and the patient to be treated. Typically, for each disease indication an optimal dosage will be determined specifying the amount to be administered per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment.

Useful dosages of the labelled compound thereof as envisaged herein can be determined by determining their in vitro activity, and/or in vivo activity in animal models.

In certain embodiments, the combinations as disclosed herein are for use in the prevention and/or treatment of cancer (preferably cancer which is associated with the expression of human FAP on cancer cells and/or on CAF) by a subsequent administration of the labelled compound at a dose ranging from 10 μg and 10 mg or from 10 μg and 7 mg or from 10 μg and 5 mg or from 10 μg and 2 mg or from 10 μg and 1.5 mg or from 10 μg and 1 mg of the labelled compound. In further particular embodiments, the labelled compound is administered at a dose ranging from 10 μg and 2 mg of labelled compound, such as in particular ranging from 10 μg and 1.5 mg or ranging from 100 μg and 1 mg of labelled compound.

Accordingly, the dose of radioactivity applied to the patient per administration has to be high enough to be effective but must be below the dose limiting toxicity (DLT). For combinations according to the invention, e.g. with 131-Iodine, the maximally tolerated dose (MTD) has to be determined which must not be exceeded in therapeutic settings.

The first and the subsequent administration are preferably according to a regimen of treatment that is suitable for preventing and/or treating the disease or disorder to be prevented or treated. The clinician will generally be able to determine a suitable treatment regimen. Generally, the treatment regimen will comprise the administration of a labelled compound, or of one or more compositions comprising the same, in one or more pharmaceutically effective amounts or doses.

The desired dose may conveniently be presented in a single dose or as divided doses (which can again be sub-dosed) administered at appropriate intervals. An administration regimen could include long-term (i.e., at least two weeks, and for example several months or years) or daily treatment. In particular, an administration regimen can vary between once a day to once a month, such as between once a day and once every two weeks, such as but not limited to once a week. Thus, depending on the desired duration and effectiveness of the treatment, labelled compound or composition comprising the same as disclosed herein may be administered once or several times, also intermittently, for instance on a daily basis for several days, weeks or months and in different dosages. The amount applied of the labelled compound or composition disclosed herein depends on the nature of the particular cancer disease. Multiple administrations are preferred. However, radiolabelled materials are typically administered at intervals of 1 to 20 weeks apart or 2 to 10 weeks apart or 2 to 8 weeks apart or 3 to 6 weeks apart or 3 to 5 weeks apart or each 4 weeks. The skilled artisan knows however how to choose dividing the administration into two or more applications, which may be applied shortly after each other, or at some other predetermined interval ranging e.g. from 1 day to 4 weeks.

In particular, the combinations, and correspondingly the labelled compounds, as envisaged herein may be used in combination with other pharmaceutically active compounds or principles that are or can be used for the prevention and/or treatment of the diseases and disorders cited herein, as a result of which a synergistic effect may or may not be obtained. Examples of such compounds and principles, as well as routes, methods and pharmaceutical formulations or compositions for administering them will be clear to the clinician.

In the context of this invention, “in combination with”, “in combination therapy” or “in combination treatment” shall mean that the combinations according to the invention are applied together with one or more other pharmaceutically active compounds or principles to the patient in a regimen wherein the patient may profit from a beneficial effect. In particular, both treatments are applied to the patient in temporal proximity. In a preferred embodiment, both treatments are applied to the patient within four weeks (28 days). More preferably, both treatments are applied within two weeks (14 days), more preferred within one week (7 days). In a preferred embodiment, the two treatments are applied within two or three days. In another preferred embodiment, the two treatments are applied at the same day, i.e. within 24 hours. In another embodiment, the two treatments are applied within four hours, or two hours, or within one hour. In another embodiment, the two treatments are applied in parallel, i.e. at the same time, or the two administrations are overlapping in time.

In particular non-limiting embodiments, the combinations according to the invention are applied together with a molecule or a composition comprising it, wherein said molecule or composition comprising it is able to optimize and therefore reduce kidney retention of the label comprised in said combination. In the context of the invention, “applied together with” is to be construed broadly. It means it encompasses applied simultaneously on one or in two distinct compositions. It also encompasses applied sequentially in two distinct compositions.

Such a molecule may be a plasma or blood substitute such as modified gelatin. An example of modified gelatin that may be used in this context is Gelofusine™. The use of such plasma or blood substitute is expected to optimize and therefore reduce the retention of the labelled compound in the kidney and therefore to optimize unwanted side effects.

Another example of such a molecule may be a positively charged amino acid or a composition comprising at least one positively charged amino acid. Examples of suitable positively charged amino acids are arginine, lysine and/or histidine. An example of such a composition is Aminomedix™. The use of positively charged amino acids has been extensively described in WO 2014/204854 which is explicitly incorporated by reference.

In particular non-limiting embodiments, the combinations according to the invention are applied together with immunotherapy. In an embodiment, the combinations according to the invention are applied with one or more therapeutic antibodies or therapeutic antibody fragments. Thus, in these particular non-limiting embodiments, the pretargeting radioimmunotherapy with the combinations according to the invention is combined with regular immunotherapy with one or more therapeutic antibodies or therapeutic antibody fragments. In further particular embodiments, the combinations according to the invention are used in a combined therapy or a combined treatment method with one or more therapeutic antibodies or therapeutic antibody fragments.

For example, the label comprised in a combinations according to the invention and the one or more therapeutic antibodies or therapeutic antibody fragments may be infused at the same time, or the infusions may be overlapping in time. If they are administered at the same time, they may be formulated together in one single pharmaceutical preparation, or they may be mixed together immediately before administration from two different pharmaceutical preparations, for example by dissolving or diluting into one single infusion solution. In another embodiment, the two are administered separately, i.e. as two independent pharmaceutical compositions. In one preferred embodiment, administration of the two treatments is in a way that tumour cells within the body of the patient are exposed to effective amounts of the cytotoxic drug and the radiation at the same time. In another preferred embodiment, effective amounts of both compounds (the label comprised in a combination according to the invention, the one or more therapeutic antibodies or fragments thereof) are present at the site of the tumour at the same time. The present invention also embraces the use of further agents, which are administered in addition to the combination as defined. This could be, for example, one or more further chemotherapeutic agent(s). It could also be one or more agent(s) applied to prevent, suppress, or ameliorate unwanted side effects of any of the other drugs given. For example, a cytokine stimulating proliferation of leukocytes may be applied to ameliorate the effects of leukopenia or neutropenia.

The efficacy of the combinations according to the invention, can be tested using any suitable in vitro assay, cell-based assay, in vivo assay and/or animal model known per se, or any combination thereof, depending on the specific disease or disorder involved. Suitable assays and animal models will be clear to the skilled person.

The skilled person will generally be able to select a suitable in vitro or in vivo assay, cellular assay or animal model to test a VHH, or fragment thereof, according to the invention for binding to human and/or murine FAP, as well as for their therapeutic and/or prophylactic effect in respect of one or more cancer-related diseases and fibrotic disorders. Such assay may be an imaging assay as disclosed herein.

The term ‘effective amount’, as used herein, means the amount needed to achieve the desired result or results.

As used herein, the terms ‘determining’, ‘measuring’, ‘assessing’, ‘monitoring’ and ‘assaying’ are used interchangeably and include both quantitative and qualitative determinations.

As used herein, the term ‘prevention and/or treatment’ comprises preventing and/or treating a certain disease and/or disorder and/or condition, preventing the onset of a certain disease and/or disorder and/or condition, slowing down or reversing the progress of a certain disease and/or disorder and/or condition, preventing or slowing down the onset of one or more symptoms associated with a certain disease and/or disorder and/or condition, reducing and/or alleviating one or more symptoms associated with a certain disease and/or disorder and/or condition, reducing the severity and/or the duration of a certain disease and/or disorder and/or condition, and generally any prophylactic or therapeutic effect of the combinations according to the invention that is beneficial to the subject or patient being treated.

### Cancer, Tumour, Metastatic Cell

As used herein, the term ‘tumour cell’ refers to a cell that is present in a primary or metastatic tumour lesion. In this context, tumours consist not only of cancer cells, but should be considered as organ-like structures in which a complex bidirectional interplay exists between transformed and non-transformed cells. The malignant potential of transformed cells requires an apt support structure from the stroma, which can consist of fibroblasts, adipocytes, blood and lymph vessels, but may also be considerably infiltrated by a wide range of immune cells. Within the context of the invention, a tumour cell may also be a fibroblast, preferably a CAF.

By “solid tumour(s)” or “tumour(s)” are meant primary tumours and/or metastases (wherever located). The terms solid cancer and solid tumor are used interchangeably herein. Examples of solid tumors are sarcomas, carcinomas, and lymphomas. Examples of a sarcoma are an osteosarcoma or osteogenic sarcoma, a chondrosarcoma, a leiomyosarcoma, a rhabdomyosarcoma, a mesothelial sarcoma or mesothelioma, a fibrosarcoma, an angiosarcoma or hemangioendothelioma, a liposarcoma, a glioma or astrocytoma, a myxosarcoma, and a mesenchymous or mixed mesodermal tumor. Examples of carcinomas are adenocarcinomas and squamous cell carcinomas. Examples of lymphomas are non-Hodgkin lymphomas and Hodgkin lymphomas.

As used herein, the term ‘cancer cell’ refers to a cell that divides and reproduces abnormally and limitlessly with uncontrolled growth and which can break away and travel to other parts of the body and set up another site, referred to as metastasis.

A ‘lesion’ as used herein can refer to any abnormal change in a body tissue or organ resulting from injury or disease. In cancer terminology, lesion typically refers to a tumour.

The term ‘primary tumour(s)’ as used herein is a tumour growing at the anatomical site where tumour progression began and proceeded to yield a cancerous mass.

The term ‘metastatic lesion(s)’ as used herein refers to malignant, or cancerous, tumours that have spread from their original location to other parts of the body. Related medical terms that might be used interchangeably include late-stage cancer, advanced cancer, or metastatic disease. In general, metastatic lesions are considered to be incurable, although treatment is often available to control the spread of cancerous cells and potentially increase the individual's life expectancy.

Metastasis is the term for the spread of cancer beyond its originating site in the body. Thus, metastatic lesions are cancerous tumours that are found in locations apart from the original starting point of the primary tumour. Metastatic tumours occur when cells from the primary tumour break off and travel to distant parts of the body via the lymph system and blood stream. Alternately, cells from the original tumour could seed into new tumours at adjacent organs or tissues. ‘Metastatic disease’ as used herein refers to late-stage cancer and to the medical classification of cancer as being in stage III, when cancer cells are found in lymph nodes near the original tumour, or in stage IV, when cancer cells have travelled far beyond the primary tumour site to distant parts of the body. Metastatic lesions are most commonly found in the brain, lungs, liver, or bones. An individual with metastatic cancer might or might not experience any symptoms, and the symptoms could be related to the area where metastasized cells have relocated. Once metastatic lesions are present in the body, the individual's cancer will be considered incurable for most cancer types. This means it is excessively difficult to eradicate every existing cancer cell with available treatments. In this case, the goal of treatment becomes slowing the growth of tumours to maintain the highest possible quality of life and potentially extend the individual's life expectancy. In some cases, people with metastatic lesions can live for a number of years with appropriate treatment for symptom management.

### Radionuclide

As used herein, the terms ‘radionuclide’, ‘radioactive nuclide’, ‘radioisotope’ or ‘radioactive isotope’, are used interchangeably herein and refer to atoms with an unstable nucleus, characterized by excess energy available to be imparted either to a newly created radiation particle within the nucleus or via internal conversion. During this process, the radionuclide is said to undergo radioactive decay, resulting in the emission of gamma ray(s) and/or subatomic particles such as alpha or beta particles. These emissions constitute ionizing radiation. Radionuclides occur naturally or can be produced artificially.

### IHC Techniques

The term ‘immunohistochemistry (IHC)’ as used herein refers to the process of detecting antigens (e.g., proteins) in cells of a tissue section by exploiting the principle of antibodies binding specifically to antigens in sections of biological tissues. Immunohistochemical staining is widely used in the diagnosis of abnormal cells such as those found in cancerous tumours. IHC is also widely used in basic research to understand the distribution and localization of biomarkers and differentially expressed proteins in different parts of a biological tissue.

All documents cited in the present specification are hereby incorporated by reference in their entirety. Unless otherwise defined, all terms used in disclosing the invention, including technical and scientific terms, have the meaning as commonly understood by one of ordinary skill in the art to which this invention belongs. By means of further guidance, term definitions are included to better appreciate the teaching of the present invention. Each embodiment described herein may be combined together with any other embodiment described herein, unless otherwise indicated.

The present invention is further described by the following examples which should not be construed as limiting the scope of the invention.

### EXAMPLES

In the examples, the following VHHs are used:

| Name | Target | SEQ ID NO |
|---|---|---|
| VHH1 | FAP | 4 |
| VHH2 | FOLR1 | 21 |
| VHH3 | FOLR1 | 22 |
| VHH4 | HER2 | 46 |
| VHH5 | HER2 | 47 |
| VHH6 | FAP | 89 |
| VHH7 | FAP | 90 |
| VHH8 | FAP | 91 |
| VHH9 | FAP | 92 |

### Example 1: Optimization of the Lag Time for ¹⁷⁷Lu-VHH1 Using a Pre-Targeting Strategy

This example describes the biodistribution of ¹⁷⁷Lu-VHH1 using a pre-targeting strategy, in which VHH1, functionalized with transcyclooctene (TCO), and ¹⁷⁷Lu-DOTA-PEG₇-Tz are sequentially administered intravenously (i.v.) to healthy C57BL/6 mice. Here we assessed potential differences in organ and tissue uptake when considering different lag times between the sequential administration of the two compounds: (1) TCO-VHH1 and (2) ¹⁷⁷Lu-DOTA-PEG₇-Tz. To this, healthy C57BL/6 mice were i.v. administered first with 50 μg (4 nmol) TCO-VHH1, followed by 50 μCi (±5 μg, 2 nmol) ¹⁷⁷Lu-DOTA-PEG₇-Tz. Four different lag times (30 min, 2, 8 and 24 hours) were evaluated. The resulting biodistributions were compared to those obtained for ¹⁷⁷Lu-DOTA-PEG₇-Tz alone and ¹⁷⁷Lu-DOTA-VHH1. Next, mice were euthanized by cervical dislocation up to 4 h post injection, dissected, after which different organs and tissues were collected. Organs and tissues of interest were weighed and measured for radioactivity using an automatic gamma counter, along with injection standards. Results were expressed as % of injected activity (IA)/g tissue.

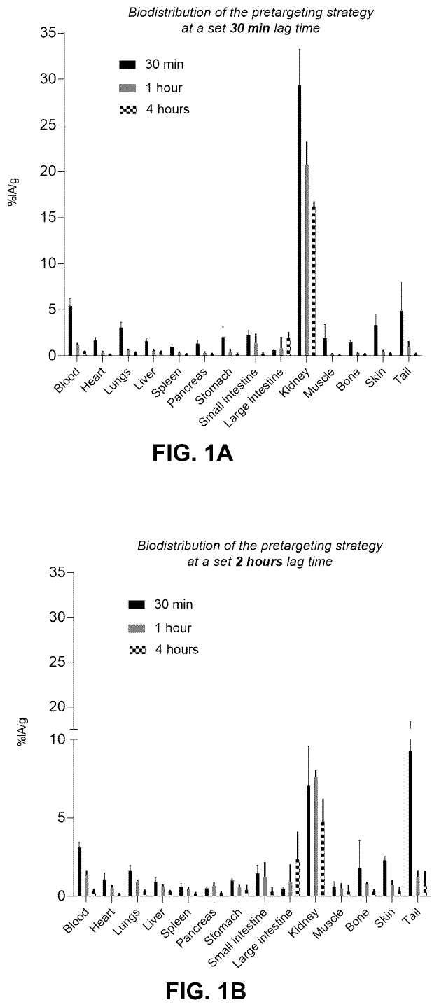

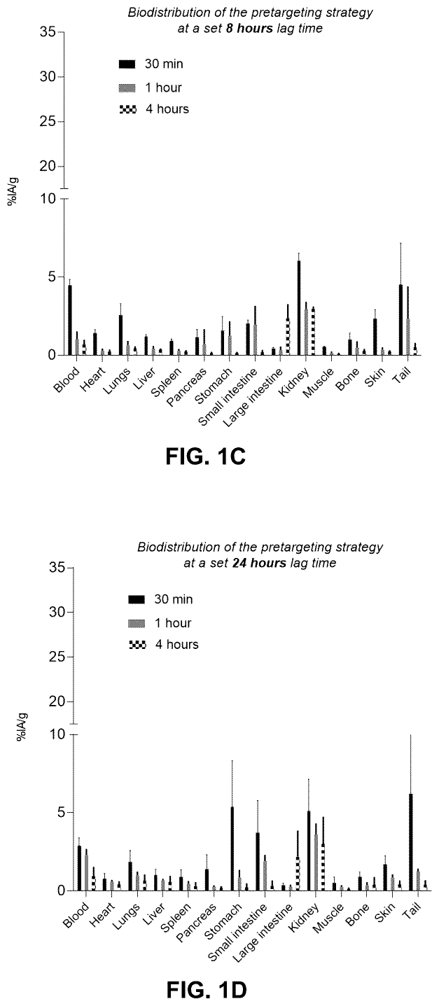

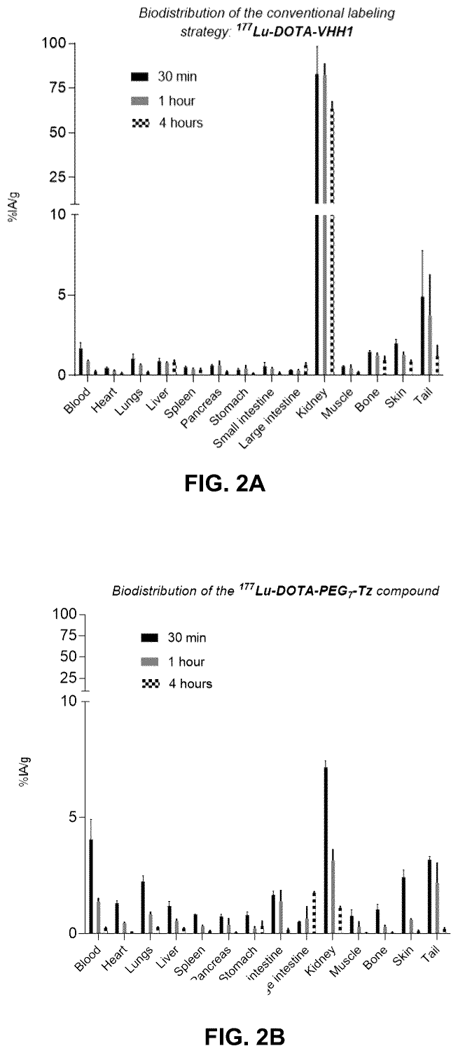

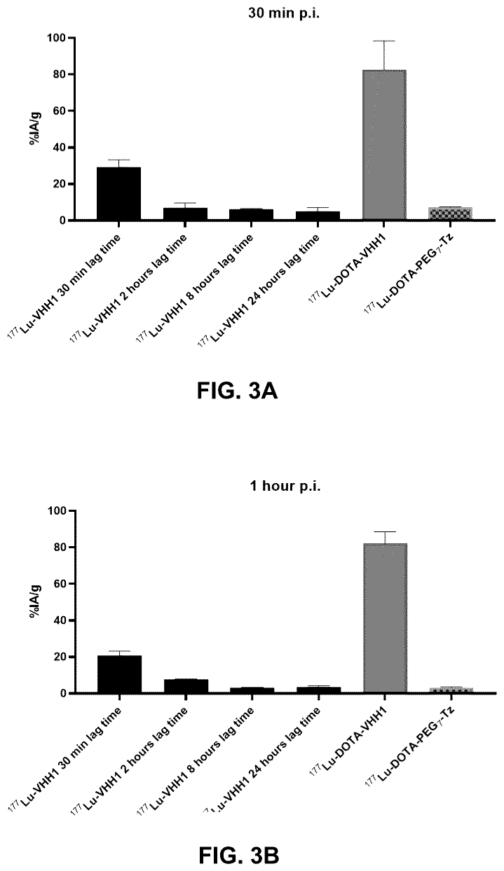

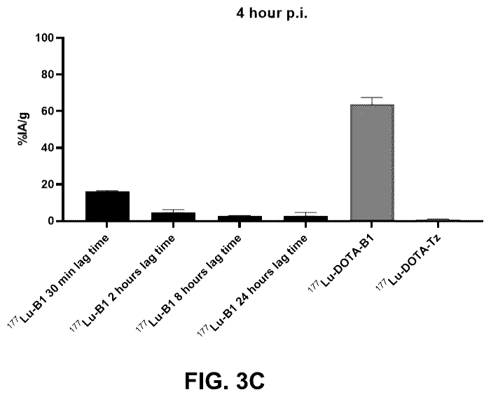

The use of the pre-targeting strategy resulted in a significant decrease (P<0.0001) in kidney retention of ¹⁷⁷Lu-VHH1 with all lag times evaluated (Table 6, FIG. 1.A-D & FIG. 3.A-C) compared to that of ¹⁷⁷Lu-DOTA-VHH1 (Table 7 & FIG. 2.A). The administration of ¹⁷⁷Lu-DOTA-VHH1 results in a kidney uptake of 82.8±15.7% IA/g after 1 h, while with a pre-targeting strategy with a 30 min lag time, a 3-fold reduction of kidney uptake was observed (29.4±3.9% IA/g at 1 h p.i.). Longer lag times (2, 8, and 24 h) further reduced the radioactive amount retained in kidneys. A longer lag time (2, 8 and 24 h) results in a significantly (P<0.0001) lower kidney retention compared to the shorter lag time of 30 min (FIG. 3.A-C). In the case of lag times 2, 8 and 24 h, a 10-fold reduction of kidney uptake was observed compared to the uptake in kidneys observed for ¹⁷⁷Lu-DOTA-VHH1. No significant difference (P>0.59) in kidney retention of ¹⁷⁷Lu-VHH1 using a pre-targeting strategy was observed between the latter lag times (FIG. 3.A-C). More so, the uptake in kidneys, when applying lag times between 2-24 h, was similar (P>0.59) to that of ¹⁷⁷Lu-DOTA-PEG₇-Tz alone (Table 8, FIG. 2.VHH1 & FIG. 3.A-C). Based on this observation it seems that with a lag time of 2 h and beyond, TCO-VHH1 is no longer available at relevant amounts in kidneys to interact with ¹⁷⁷Lu-DOTA-PEG₇-Tz. Based on these results it seems that an optimal lag time between the administrations of TCO-VHH1 and ¹⁷⁷Lu-DOTA-PEG₇-Tz to reduce retention of ¹⁷⁷Lu-VHH1 using a pre-targeting strategy in kidneys is defined between 2 and 8 h.

TABLE 6
Uptake values in different organs and tissues for ¹⁷⁷Lu-VHH1 using
a pre-targeting strategy with lag times of 30 min, 2, 8 and 24 h. Mice
were evaluated up to 4 h post i.v. injection of ¹⁷⁷Lu-DOTA-PEG₇-Tz.
Data is expressed as % injected activity per gram of tissue (%
IA/g) and presented as mean ± SD (n = 5).
Table 6(a): 30 min lag time

| | 30 min | | 1 h | | 4 h | |
|---|---|---|---|---|---|---|
| Organ/tissue | MEAN | SD | MEAN | SD | MEAN | SD |
| Blood | 5.43 | 0.79 | 1.24 | 0.08 | 0.49 | 0.02 |
| Heart | 1.70 | 0.30 | 0.34 | 0.13 | 0.17 | 0.01 |
| Lungs | 3.06 | 0.58 | 0.57 | 0.13 | 0.36 | 0.05 |
| Liver | 1.55 | 0.36 | 0.56 | 0.04 | 0.45 | 0.04 |
| Spleen | 1.01 | 0.22 | 0.34 | 0.07 | 0.19 | 0.04 |
| Pancreas | 1.35 | 0.37 | 0.33 | 0.09 | 0.19 | 0.06 |
| Stomach | 2.05 | 1.09 | 0.40 | 0.30 | 0.19 | 0.09 |
| Small intestine | 2.29 | 0.47 | 1.37 | 0.96 | 0.23 | 0.09 |
| Large intestine | 0.59 | 0.13 | 0.83 | 1.16 | 1.95 | 0.57 |
| Kidney | 29.37 | 3.85 | 20.74 | 2.39 | 16.14 | 0.53 |
| Muscle | 1.91 | 1.50 | 0.22 | 0.02 | 0.10 | 0.02 |
| Bone | 1.48 | 0.20 | 0.31 | 0.05 | 0.18 | 0.06 |
| Skin | 3.33 | 1.19 | 0.49 | 0.08 | 0.29 | 0.06 |
| Tail | 4.92 | 3.11 | 0.98 | 0.54 | 0.26 | 0.02 |

Table 6(b): 2 hours lag time

| | 30 min | | 1 h | | 4 h | |
|---|---|---|---|---|---|---|
| Organ/tissue | MEAN | SD | MEAN | SD | MEAN | SD |
| Blood | 3.10 | 0.34 | 1.38 | 0.20 | 0.38 | 0.05 |
| Heart | 1.10 | 0.38 | 0.55 | 0.12 | 0.13 | 0.04 |
| Lungs | 1.62 | 0.36 | 0.97 | 0.06 | 0.29 | 0.10 |
| Liver | 0.93 | 0.25 | 0.65 | 0.06 | 0.29 | 0.06 |
| Spleen | 0.61 | 0.21 | 0.48 | 0.09 | 0.18 | 0.05 |
| Pancreas | 0.53 | 0.06 | 0.67 | 0.21 | 0.21 | 0.06 |
| Stomach | 1.01 | 0.11 | 0.56 | 0.14 | 0.39 | 0.30 |
| Small intestine | 1.48 | 0.51 | 1.23 | 0.91 | 0.28 | 0.25 |
| Large intestine | 0.46 | 0.08 | 0.92 | 1.08 | 2.36 | 1.72 |
| Kidney | 7.07 | 2.52 | 7.61 | 0.40 | 4.74 | 1.44 |
| Muscle | 0.61 | 0.32 | 0.50 | 0.30 | 0.31 | 0.37 |
| Bone | 1.81 | 1.76 | 0.78 | 0.12 | 0.28 | 0.12 |
| Skin | 2.31 | 0.25 | 0.71 | 0.31 | 0.35 | 0.22 |
| Tail | 9.29 | 9.05 | 1.20 | 0.38 | 0.84 | 0.71 |

Table 6(c): 8 hours lag time

| | 30 min | | 1 h | | 4 h | |
|---|---|---|---|---|---|---|
| Organ/tissue | MEAN | SD | MEAN | SD | MEAN | SD |
| Blood | 4.49 | 0.37 | 1.04 | 0.45 | 0.70 | 0.25 |
| Heart | 1.44 | 0.20 | 0.30 | 0.09 | 0.24 | 0.09 |
| Lungs | 2.59 | 0.72 | 0.65 | 0.22 | 0.45 | 0.08 |
| Liver | 1.19 | 0.14 | 0.43 | 0.13 | 0.37 | 0.03 |
| Spleen | 0.92 | 0.13 | 0.29 | 0.07 | 0.25 | 0.03 |
| Pancreas | 1.17 | 0.49 | 0.72 | 0.91 | 0.15 | 0.03 |
| Stomach | 1.58 | 0.91 | 1.26 | 0.88 | 0.15 | 0.03 |
| Small intestine | 2.04 | 0.22 | 1.97 | 1.15 | 0.20 | 0.10 |
| Large intestine | 0.44 | 0.08 | 0.34 | 0.18 | 2.38 | 0.84 |
| Kidney | 6.07 | 0.46 | 2.95 | 0.43 | 2.98 | 0.10 |
| Muscle | 0.54 | 0.05 | 0.14 | 0.06 | 0.09 | 0.03 |
| Bone | 1.01 | 0.41 | 0.49 | 0.37 | 0.28 | 0.09 |
| Skin | 2.35 | 0.57 | 0.35 | 0.12 | 0.25 | 0.03 |
| Tail | 4.51 | 2.67 | 2.35 | 2.01 | 0.54 | 0.21 |

Table 6(d): 24 hours lag time

| | 30 min | | 1 h | | 4 h | |
|---|---|---|---|---|---|---|
| Organ/tissue | MEAN | SD | MEAN | SD | MEAN | SD |
| Blood | 2.87 | 0.52 | 2.31 | 0.33 | 0.96 | 0.53 |
| Heart | 0.77 | 0.35 | 0.60 | 0.06 | 0.41 | 0.16 |
| Lungs | 1.85 | 0.74 | 1.00 | 0.19 | 0.66 | 0.34 |
| Liver | 0.99 | 0.39 | 0.67 | 0.06 | 0.57 | 0.35 |
| Spleen | 0.91 | 0.45 | 0.46 | 0.13 | 0.31 | 0.20 |
| Pancreas | 1.41 | 0.91 | 0.28 | 0.03 | 0.21 | 0.05 |
| Stomach | 5.35 | 2.98 | 0.85 | 0.45 | 0.22 | 0.22 |
| Small intestine | 3.71 | 2.07 | 1.91 | 0.34 | 0.32 | 0.30 |
| Large intestine | 0.36 | 0.13 | 0.27 | 0.08 | 2.18 | 1.63 |
| Kidney | 5.09 | 2.05 | 3.62 | 0.65 | 3.00 | 1.70 |
| Muscle | 0.53 | 0.35 | 0.25 | 0.07 | 0.12 | 0.05 |
| Bone | 0.89 | 0.31 | 0.36 | 0.15 | 0.39 | 0.45 |
| Skin | 1.69 | 0.56 | 0.85 | 0.16 | 0.41 | 0.21 |
| Tail | 6.22 | 4.56 | 1.25 | 0.12 | 0.43 | 0.21 |

TABLE 7
Uptake values in different organs and tissues for
¹⁷⁷Lu-DOTA-VHH1 up to 4 h post i.v. injection. Data
is expressed as % injected activity per gram of tissue
(% IA/g) and presented as mean ± SD (n = 5).

| | 30 min | | 1 h | | 4 h | |
|---|---|---|---|---|---|---|
| Organ/tissue | MEAN | SD | MEAN | SD | MEAN | SD |
| Blood | 1.69 | 0.37 | 0.89 | 0.06 | 0.26 | 0.06 |
| Heart | 0.47 | 0.07 | 0.32 | 0.02 | 0.15 | 0.04 |
| Lungs | 1.04 | 0.30 | 0.66 | 0.05 | 0.23 | 0.04 |
| Liver | 0.88 | 0.20 | 0.81 | 0.04 | 0.83 | 0.14 |
| Spleen | 0.54 | 0.05 | 0.41 | 0.05 | 0.36 | 0.08 |
| Pancreas | 0.64 | 0.06 | 0.63 | 0.26 | 0.25 | 0.04 |
| Stomach | 0.38 | 0.07 | 0.45 | 0.17 | 0.14 | 0.02 |
| Small intestine | 0.57 | 0.26 | 0.45 | 0.05 | 0.18 | 0.04 |
| Large intestine | 0.33 | 0.04 | 0.31 | 0.06 | 0.66 | 0.15 |
| Kidney | 82.75 | 15.68 | 82.36 | 6.31 | 63.74 | 3.79 |
| Muscle | 0.58 | 0.04 | 0.45 | 0.21 | 0.21 | 0.04 |
| Bone | 1.48 | 0.09 | 1.27 | 0.13 | 0.95 | 0.25 |
| Skin | 1.99 | 0.26 | 1.30 | 0.16 | 0.87 | 0.09 |
| Tail | 4.89 | 2.90 | 3.74 | 2.52 | 1.21 | 0.67 |

TABLE 8
Uptake values in different organs and tissues for ¹⁷⁷Lu-DOTA-PEG₇-
Tz up to 4 h post i.v. injection, expressed as % injected activity per
gram of tissue (% IA/g). Data presented as mean ± SD (n = 5).

| | 30 min | | 1 h | | 4 h | |
|---|---|---|---|---|---|---|
| Organ/tissue | MEAN | SD | MEAN | SD | MEAN | SD |
| Blood | 4.06 | 0.87 | 1.39 | 0.13 | 0.25 | 0.03 |
| Heart | 1.31 | 0.11 | 0.46 | 0.03 | 0.10 | 0.00 |
| Lungs | 2.24 | 0.26 | 0.84 | 0.09 | 0.28 | 0.01 |
| Liver | 1.19 | 0.21 | 0.56 | 0.07 | 0.24 | 0.01 |
| Spleen | 0.82 | 0.02 | 0.32 | 0.04 | 0.11 | 0.03 |
| Pancreas | 0.73 | 0.11 | 0.39 | 0.26 | 0.06 | 0.01 |
| Stomach | 0.79 | 0.15 | 0.22 | 0.08 | 0.34 | 0.20 |
| Small intestine | 1.66 | 0.18 | 1.40 | 0.46 | 0.17 | 0.05 |
| Large intestine | 0.51 | 0.03 | 0.66 | 0.50 | 1.76 | 0.05 |
| Kidney | 7.18 | 0.27 | 3.15 | 0.47 | 1.12 | 0.06 |
| Muscle | 0.77 | 0.27 | 0.30 | 0.21 | 0.04 | 0.01 |
| Bone | 1.03 | 0.24 | 0.30 | 0.06 | 0.06 | 0.01 |
| Skin | 2.43 | 0.32 | 0.61 | 0.03 | 0.11 | 0.04 |
| Tail | 3.18 | 0.15 | 2.20 | 0.84 | 0.21 | 0.05 |

### Example 2: Optimization of the Administered Mass of TCO-VHH1 Using a Pre-Targeting Strategy

This example describes the biodistribution of ¹⁷⁷Lu-VHH1 using a pre-targeting strategy, in which VHH1, functionalized with transcyclooctene (TCO), and ¹⁷⁷Lu-DOTA-tetrazine (Tz) are sequentially administered intravenously (i.v.) to healthy C57BL/6 mice. Here we assessed potential differences in organ and tissue uptake when varying the administered mass of TCO-VHH1. To this, healthy C57BL/6 mice were i.v. injected with TCO-VHH1 at four different masses: Oct. 20, 1950-200 μg, corresponding to 0.8-1.6-4-16 nmol. Eight h after i.v. administration of TCO-VHH1, the animals were treated with Oct. 20, 1950-200 μCi (corresponding to 0.4-0.8-2-8 nmol) of ¹⁷⁷Lu-DOTA-PEG₇-Tz. The resulting biodistributions of ¹⁷⁷Lu-VHH1 were compared to that of ¹⁷⁷Lu-DOTA-PEG₇-Tz 50 μCi (±2 nmol) alone. Next, the mice were euthanized by cervical dislocation up to 4 h post injection, dissected, after which different organs and tissues were collected. Organs and tissues of interest were weighed and measured for radioactivity using an automatic gamma counter, along with injection standards. Results were expressed as % of injected activity (IA)/g tissue.

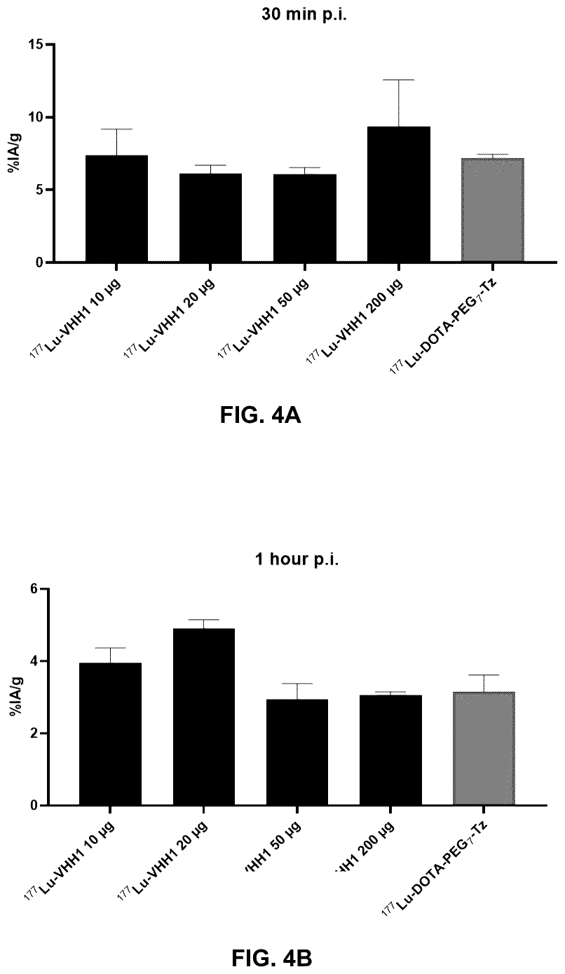

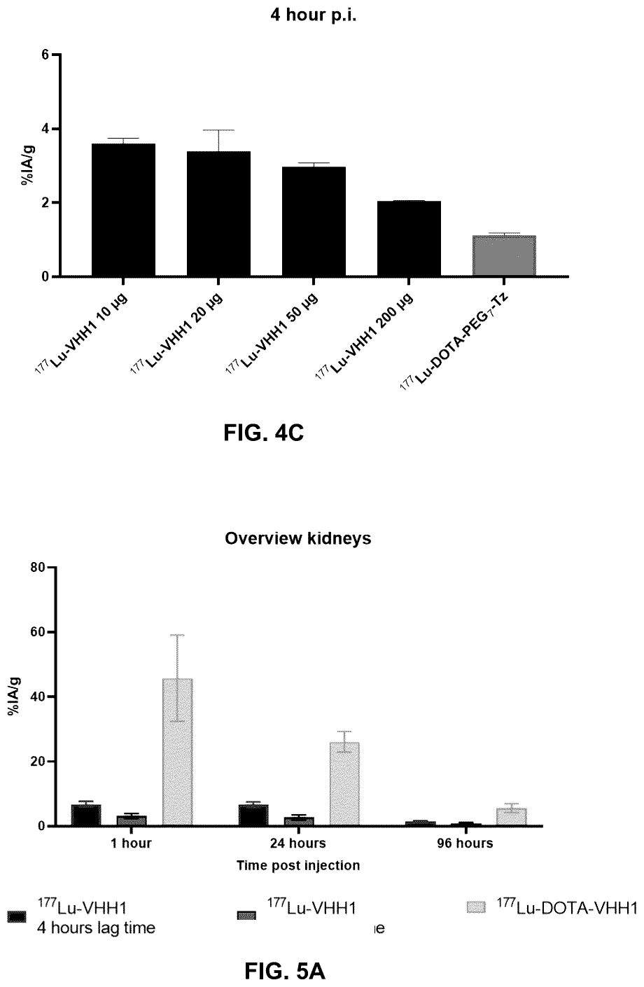

Varying the administered mass of TCO-VHH1 (10-200 μg) did not impact the radioactive uptake in the kidneys (FIG. 4.A) at 30 min p.i. For example, TCO-VHH1 (10-200 μg) followed by ¹⁷⁷Lu-DOTA-PEG₇-Tz (with 8 h lag time) always resulted in a kidney uptake of about 7% IA/g 30 min after injection of the ¹⁷⁷Lu-DOTA-PEG₇-Tz, as shown in Table 9. Moreover, these were similar to that obtained for ¹⁷⁷Lu-DOTA-PEG₇-Tz alone (Table 10; 7.0±0.27% IA/g) at 30 min p.i. Starting from 1 h p.i., a significantly lower kidney uptake was observed for higher a higher injected mass of TCO-VHH1 (e.g. 50-200 μg) compared to the injection of lower masses of TCO-VHH1 (e.g. 10-20 μg) (P<0.0001) (FIG. 4.B.C). These data indicate that a higher administered mass of TCO-VHH1 does impact the kidney uptake of the resulting ¹⁷⁷Lu-VHH1 using a pre-targeting strategy.

TABLE 9
Uptake values in different organs and tissues for ¹⁷⁷Lu-VHH1
using a pre-targeting strategy evaluating four different
masses of TCO-VHH1 (10, 20, 50 and 200 μg). The mice were evaluated
up to 4 h post i.v. injection of ¹⁷⁷Lu-DOTA-PEG₇-Tz. Data
is expressed as % injected activity per gram of tissue (%
IA/g) and presented as mean ± SD (n = 5).
Table 9(a): 10 μg

| | 30 min | | 1 h | | 4 h | |
|---|---|---|---|---|---|---|
| Organ/tissue | MEAN | SD | MEAN | SD | MEAN | SD |
| Blood | 4.22 | 0.75 | 1.19 | 0.55 | 0.64 | 0.15 |
| Heart | 1.10 | 0.22 | 0.61 | 0.31 | 0.15 | 0.03 |
| Lungs | 2.21 | 0.48 | 0.90 | 0.19 | 0.38 | 0.05 |
| Liver | 1.21 | 0.17 | 0.57 | 0.09 | 0.42 | 0.01 |
| Spleen | 0.71 | 0.07 | 0.56 | 0.16 | 0.26 | 0.06 |
| Pancreas | 0.77 | 0.22 | 0.96 | 0.31 | 0.27 | 0.01 |
| Stomach | 1.21 | 0.14 | 0.34 | 0.01 | 0.14 | 0.00 |
| Small intestine | 2.16 | 0.38 | 1.50 | 0.48 | 0.19 | 0.03 |
| Large intestine | 0.50 | 0.17 | 0.35 | 0.13 | 2.98 | 0.27 |
| Kidney | 7.36 | 1.82 | 3.95 | 0.42 | 3.60 | 0.15 |
| Muscle | 0.78 | 0.10 | 0.44 | 0.26 | 0.08 | 0.03 |
| Bone | 1.07 | 0.24 | 0.77 | 0.23 | 0.18 | 0.06 |
| Skin | 2.97 | 1.16 | 0.84 | 0.40 | 0.26 | 0.16 |
| Tail | 3.96 | 0.69 | 2.09 | 1.99 | 0.47 | 0.11 |

Table 9(b): 20 μg

| | 30 min | | 1 h | | 4 h | |
|---|---|---|---|---|---|---|
| Organ/tissue | MEAN | SD | MEAN | SD | MEAN | SD |
| Blood | 3.84 | 0.11 | 2.73 | 0.33 | 0.48 | 0.19 |
| Heart | 0.99 | 0.15 | 0.56 | 0.07 | 0.17 | 0.03 |
| Lungs | 1.77 | 0.25 | 1.12 | 0.06 | 0.42 | 0.01 |
| Liver | 1.18 | 0.14 | 0.80 | 0.06 | 0.47 | 0.01 |
| Spleen | 0.65 | 0.06 | 0.52 | 0.16 | 0.18 | 0.02 |
| Pancreas | 0.55 | 0.01 | 0.48 | 0.02 | 0.11 | 0.03 |
| Stomach | 0.51 | 0.08 | 0.49 | 0.23 | 0.12 | 0.05 |
| Small intestine | 1.58 | 0.39 | 1.69 | 0.10 | 0.19 | 0.02 |
| Large intestine | 0.38 | 0.03 | 0.25 | 0.02 | 2.89 | 0.15 |
| Kidney | 6.15 | 0.56 | 4.91 | 0.24 | 3.39 | 0.57 |
| Muscle | 0.59 | 0.01 | 0.42 | 0.10 | 0.11 | 0.04 |
| Bone | 0.67 | 0.03 | 0.92 | 0.02 | 0.20 | 0.03 |
| Skin | 1.80 | 0.20 | 0.85 | 0.22 | 0.29 | 0.07 |
| Tail | 2.50 | 0.17 | 1.24 | 0.26 | 0.47 | 0.20 |

Table 9(c): 50 μg

| | 30 min | | 1 h | | 4 h | |
|---|---|---|---|---|---|---|
| Organ/tissue | MEAN | SD | MEAN | SD | MEAN | SD |
| Blood | 4.49 | 0.37 | 1.04 | 0.45 | 0.70 | 0.25 |
| Heart | 1.44 | 0.20 | 0.30 | 0.09 | 0.24 | 0.09 |
| Lungs | 2.59 | 0.72 | 0.65 | 0.22 | 0.45 | 0.08 |
| Liver | 1.19 | 0.14 | 0.43 | 0.13 | 0.37 | 0.03 |
| Spleen | 0.92 | 0.13 | 0.29 | 0.07 | 0.25 | 0.03 |
| Pancreas | 1.17 | 0.49 | 0.72 | 0.91 | 0.15 | 0.03 |
| Stomach | 1.58 | 0.91 | 1.26 | 0.88 | 0.15 | 0.03 |
| Small intestine | 2.04 | 0.22 | 1.97 | 1.15 | 0.20 | 0.10 |
| Large intestine | 0.44 | 0.08 | 0.34 | 0.18 | 2.38 | 0.84 |
| Kidney | 6.07 | 0.46 | 2.95 | 0.43 | 2.98 | 0.10 |
| Muscle | 0.54 | 0.05 | 0.14 | 0.06 | 0.09 | 0.03 |
| Bone | 1.01 | 0.41 | 0.49 | 0.37 | 0.28 | 0.09 |
| Skin | 2.35 | 0.57 | 0.35 | 0.12 | 0.25 | 0.03 |
| Tail | 4.51 | 2.67 | 2.35 | 2.01 | 0.54 | 0.21 |

Table 9(d): 200 μg

| | 30 min | | 1 h | | 4 h | |
|---|---|---|---|---|---|---|
| Organ/tissue | MEAN | SD | MEAN | SD | MEAN | SD |
| Blood | 6.28 | 0.63 | 2.36 | 0.67 | 0.85 | 0.22 |
| Heart | 1.63 | 0.59 | 0.48 | 0.04 | 0.24 | 0.05 |
| Lungs | 2.95 | 0.57 | 0.97 | 0.05 | 0.47 | 0.03 |
| Liver | 1.59 | 0.32 | 0.57 | 0.05 | 0.38 | 0.07 |
| Spleen | 1.00 | 0.23 | 0.35 | 0.01 | 0.21 | 0.01 |
| Pancreas | 0.89 | 0.23 | 0.30 | 0.03 | 0.11 | 0.01 |
| Stomach | 2.88 | 2.08 | 0.57 | 0.27 | 0.14 | 0.02 |
| Small intestine | 1.74 | 0.37 | 1.53 | 0.21 | 0.20 | 0.06 |
| Large intestine | 0.62 | 0.19 | 0.17 | 0.01 | 1.87 | 0.11 |
| Kidney | 9.35 | 3.22 | 3.06 | 0.09 | 2.05 | 0.01 |
| Muscle | 0.79 | 0.18 | 0.18 | 0.04 | 0.09 | 0.02 |
| Bone | 0.82 | 0.12 | 0.32 | 0.09 | 0.13 | 0.02 |
| Skin | 2.62 | 0.37 | 0.53 | 0.04 | 0.28 | 0.01 |
| Tail | 6.63 | 4.26 | 1.15 | 0.28 | 0.37 | 0.08 |

TABLE 10
Uptake values in different organs and tissues for ¹⁷⁷Lu-DOTA-PEG₇-
Tz up to 4 h post i.v. injection, expressed as % injected activity per
gram of tissue (% IA/g). Data presented as mean ± SD (n = 5).

| | 30 min | | 1 h | | 4 h | |
|---|---|---|---|---|---|---|
| Organ/tissue | MEAN | SD | MEAN | SD | MEAN | SD |
| Blood | 4.06 | 0.87 | 1.39 | 0.13 | 0.25 | 0.03 |
| Heart | 1.31 | 0.11 | 0.46 | 0.03 | 0.10 | 0.00 |
| Lungs | 2.24 | 0.26 | 0.84 | 0.09 | 0.28 | 0.01 |
| Liver | 1.19 | 0.21 | 0.56 | 0.07 | 0.24 | 0.01 |
| Spleen | 0.82 | 0.02 | 0.32 | 0.04 | 0.11 | 0.03 |
| Pancreas | 0.73 | 0.11 | 0.39 | 0.26 | 0.06 | 0.01 |
| Stomach | 0.79 | 0.15 | 0.22 | 0.08 | 0.34 | 0.20 |
| Small intestine | 1.66 | 0.18 | 1.40 | 0.46 | 0.17 | 0.05 |
| Large intestine | 0.51 | 0.03 | 0.66 | 0.50 | 1.76 | 0.05 |
| Kidney | 7.18 | 0.27 | 3.15 | 0.47 | 1.12 | 0.06 |
| Muscle | 0.77 | 0.27 | 0.30 | 0.21 | 0.04 | 0.01 |
| Bone | 1.03 | 0.24 | 0.30 | 0.06 | 0.06 | 0.01 |
| Skin | 2.43 | 0.32 | 0.61 | 0.03 | 0.11 | 0.04 |
| Tail | 3.18 | 0.15 | 2.20 | 0.84 | 0.21 | 0.05 |

### Example 3: Long-Term Biodistribution and Tumor Targeting of ¹⁷⁷Lu-VHH1 Using a Pre-Targeting Strategy

This example describes the tumor targeting potential of ¹⁷⁷Lu-VHH1 using a pre-targeting strategy using the TCO-VHH1 and the ¹⁷⁷Lu-DOTA-PEG₇-Tz which was evaluated over 4 days post i.v. injection in mice with human glioblastoma tumors (U-87 MG) that naturally express human FAP. To this, athymic nude mice (n=5 per time point) were inoculated subcutaneously with human FAP-expressing U-87 MG cells in the neck. After validation of tumor growth (150-250 mm³), mice were i.v. injected first with 200 μg (16 nmol) TCO-VHH1 followed by about 80 μCi (±5 μg) of ¹⁷⁷Lu-DOTA-PEG₇-Tz. Two different lag times of 4 and 8 h were assessed in parallel. The resulting biodistributions of ¹⁷⁷Lu-VHH1 using a pre-targeting strategy were compared with that obtained with ¹⁷⁷Lu-DOTA-VHH1 (80 μCi; ±5 μg). Next, the mice were euthanized by cervical dislocation up to 96 h post injection, dissected, after which different organs and tissues were collected, weighed and subsequently measured for radioactivity using an automatic gamma counter, along with injection standards. Results were expressed as % of injected activity (IA)/g tissue.

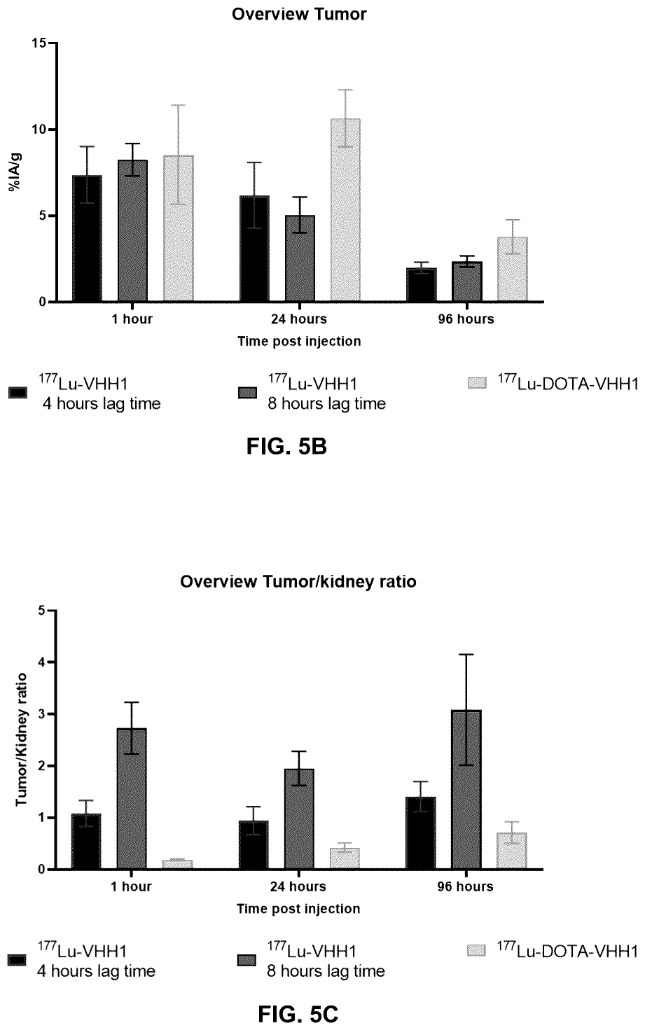

The radioactive amount in kidneys was always significantly lower (P<0.0001) for ¹⁷⁷Lu-VHH1 using a pre-targeting strategy (Table 11 and 12) compared to the biodistribution obtained with ¹⁷⁷Lu-DOTA-VHH1 (Table 13), with an uptake value of 6.76±0.87 at 1 h p.i. for ¹⁷⁷Lu-VHH1 (4 h lag time) and 3.12±0.75 at 1 h p.i. for ¹⁷⁷Lu-VHH1 (8 h lag time) versus 45.70±13.40% IA/g at the same time point for ¹⁷⁷Lu-DOTA-VHH1 (FIG. 5.A). Uptake in kidneys was lower at all time points, but not significantly different (P>0.384), for 177 Lu-VHH1 (8 h lag time) compared to the values obtained for ¹⁷⁷Lu-VHH1 (4 h lag time) using a pretargeting strategy (FIG. 5.A).

Uptake in tumor measured 7.36±1.64% IA/g after 1 h for ¹⁷⁷Lu-VHH1 (4 h lag time) and 8.24±0.94% IA/g after 1 h for ¹⁷⁷Lu-VHH1 (8 h lag time), which is not significantly (P=0.452) different than the value of 8.52±2.88% IA/g obtained with ¹⁷⁷Lu-DOTA-VHH1 (FIG. 5.B). Uptake in all other organs and tissues was low at all time points for ¹⁷⁷Lu-VHH1 using a pre-targeting strategy. From the obtained uptake values, the corresponding tumor-to-kidney (T/K) ratios over time were calculated (Table 11, 12 and 13). The T/K ratios obtained with ¹⁷⁷Lu-VHH1 (both 4 h and 8 h lag time) using a pre-targeting strategy were always higher than those calculated for ¹⁷⁷Lu-DOTA-VHH1 (FIG. 5.C), which reflects the beneficial effects of pre-targeting strategy on reducing kidney retention while maintaining the tumor targeting potential of ¹⁷⁷Lu-VHH1 over time. The T/K for ¹⁷⁷Lu-VHH1 with 8 h lag time was always significantly higher (P<0.0027) compared to the values obtained for ¹⁷⁷Lu-VHH1 with a 4 h lag time (FIG. 5.B).

TABLE 11
Uptake values in different organs and tissues for ¹⁷⁷Lu-VHH1 using
a pre-targeting strategy up to 96 h post i.v. injection, using
a lag time of 4 h. Values are expressed as % injected activity
per gram of tissue (% IA/g). Data presented as mean ± SD (n = 5).

| | 1 h | | 3 h | | 24 h | | 96 h | |
|---|---|---|---|---|---|---|---|---|
| Organ/tissue | MEAN | SD | MEAN | SD | MEAN | SD | MEAN | SD |
| Blood | 0.68 | 0.13 | 0.30 | 0.07 | 0.04 | 0.01 | 0.01 | 0.01 |
| Heart | 0.26 | 0.07 | 0.19 | 0.03 | 0.09 | 0.01 | 0.05 | 0.01 |
| Lungs | 0.83 | 0.07 | 0.54 | 0.14 | 0.31 | 0.05 | 0.25 | 0.04 |
| Liver | 0.43 | 0.04 | 0.30 | 0.09 | 0.24 | 0.02 | 0.16 | 0.02 |
| Spleen | 0.37 | 0.04 | 0.30 | 0.11 | 0.16 | 0.02 | 0.11 | 0.03 |
| Pancreas | 0.24 | 0.04 | 0.17 | 0.04 | 0.06 | 0.01 | 0.03 | 0.01 |
| Stomach | 0.32 | 0.14 | 0.20 | 0.05 | 0.06 | 0.01 | 0.07 | 0.01 |
| Small intestine | 0.75 | 0.21 | 0.40 | 0.14 | 0.06 | 0.01 | 0.06 | 0.01 |
| Large intestine | 0.26 | 0.04 | 0.43 | 0.08 | 0.09 | 0.01 | 0.10 | 0.02 |
| Kidney | 6.76 | 0.87 | 6.48 | 1.74 | 6.58 | 0.88 | 1.42 | 0.22 |
| Muscle | 0.30 | 0.04 | 0.17 | 0.04 | 0.08 | 0.01 | 0.03 | 0.01 |
| Bone | 1.26 | 0.88 | 0.22 | 0.12 | 0.15 | 0.05 | 0.05 | 0.02 |
| Joint | 1.17 | 0.13 | 0.69 | 0.16 | 0.37 | 0.05 | 0.12 | 0.03 |
| Skin | 0.67 | 0.04 | 0.44 | 0.08 | 0.24 | 0.02 | 0.12 | 0.01 |
| Tumor | 7.36 | 1.64 | 6.50 | 0.94 | 6.18 | 1.90 | 1.97 | 0.33 |
| Tumor-kidney ratio | 1.08 | 0.25 | 1.05 | 0.26 | 0.94 | 0.27 | 1.41 | 0.29 |

TABLE 12
Uptake values in different organs and tissues for ¹⁷⁷Lu-VHH1 using
a pre-targeting strategy up to 96 h post i.v. injection, using
a lag time of 8 h. Values are expressed as % injected activity
per gram of tissue (% IA/g). Data presented as mean ± SD (n = 5).

| | 1 h | | 3 h | | 24 h | | 96 h | |
|---|---|---|---|---|---|---|---|---|
| Organ/tissue | MEAN | SD | MEAN | SD | MEAN | SD | MEAN | SD |
| Blood | 0.59 | 0.06 | 0.36 | 0.02 | 0.05 | 0.01 | 0.01 | 0.00 |
| Heart | 0.24 | 0.04 | 0.20 | 0.11 | 0.10 | 0.03 | 0.06 | 0.02 |
| Lungs | 0.67 | 0.08 | 0.58 | 0.22 | 0.33 | 0.10 | 0.29 | 0.04 |
| Liver | 0.35 | 0.04 | 0.30 | 0.03 | 0.20 | 0.07 | 0.17 | 0.03 |
| Spleen | 0.30 | 0.04 | 0.23 | 0.07 | 0.18 | 0.07 | 0.11 | 0.01 |
| Pancreas | 0.15 | 0.03 | 0.13 | 0.03 | 0.06 | 0.02 | 0.03 | 0.01 |
| Stomach | 0.25 | 0.09 | 0.16 | 0.07 | 0.10 | 0.04 | 0.10 | 0.02 |
| Small intestine | 0.88 | 0.31 | 0.17 | 0.07 | 0.09 | 0.02 | 0.09 | 0.05 |
| Large intestine | 0.19 | 0.07 | 0.42 | 0.05 | 0.11 | 0.03 | 0.09 | 0.02 |
| Kidney | 3.12 | 0.75 | 3.57 | 0.47 | 2.65 | 0.78 | 0.82 | 0.26 |
| Muscle | 0.20 | 0.05 | 0.19 | 0.07 | 0.05 | 0.02 | 0.04 | 0.01 |
| Bone | 0.34 | 0.20 | 0.28 | 0.07 | 0.05 | 0.03 | 0.03 | 0.04 |
| Joint | 0.74 | 0.22 | 0.57 | 0.06 | 0.26 | 0.10 | 0.17 | 0.02 |
| Skin | 0.64 | 0.07 | 0.46 | 0.07 | 0.20 | 0.08 | 0.14 | 0.02 |
| Tumor | 8.24 | 0.94 | 6.72 | 0.71 | 5.04 | 1.04 | 2.34 | 0.33 |
| Tumor-kidney ratio | 2.73 | 0.50 | 1.89 | 0.18 | 1.95 | 0.33 | 3.08 | 1.07 |

TABLE 13
Uptake values in different organs and tissues for ¹⁷⁷Lu-DOTA-VHH1
up to 96 h post i.v. injection, expressed as % injected activity
per gram of tissue (% IA/g). Data presented as mean ± SD (n = 5).

| | 1 h | | 24 h | | 96 h | |
|---|---|---|---|---|---|---|
| Organ/tissue | MEAN | SD | MEAN | SD | MEAN | SD |
| Blood | 1.20 | 0.39 | 0.02 | 0.01 | 0.00 | 0.00 |
| Heart | 0.38 | 0.05 | 0.05 | 0.01 | 0.02 | 0.01 |
| Lungs | 0.66 | 0.12 | 0.05 | 0.01 | 0.01 | 0.00 |
| Liver | 0.46 | 0.04 | 0.36 | 0.10 | 0.12 | 0.02 |
| Spleen | 0.32 | 0.07 | 0.27 | 0.09 | 0.05 | 0.01 |
| Pancreas | 0.61 | 0.12 | 0.05 | 0.01 | 0.02 | 0.00 |
| Stomach | 0.57 | 0.34 | 0.04 | 0.01 | 0.02 | 0.01 |
| Small intestine | 0.46 | 0.26 | 0.08 | 0.02 | 0.02 | 0.00 |
| Large intestine | 0.28 | 0.03 | 0.21 | 0.13 | 0.04 | 0.00 |
| Kidney | 45.70 | 13.40 | 26.03 | 3.18 | 5.49 | 1.39 |
| Muscle | 0.69 | 0.18 | 0.08 | 0.03 | 0.02 | 0.01 |
| Bone | 1.15 | 0.17 | 0.34 | 0.09 | 0.08 | 0.02 |
| Joint | 2.37 | 0.36 | 0.60 | 0.09 | 0.22 | 0.09 |
| Skin | 1.88 | 0.33 | 0.33 | 0.03 | 0.06 | 0.02 |
| Tumor | 8.52 | 2.88 | 10.64 | 1.66 | 3.77 | 0.99 |
| Tumor-kidney ratio | 0.19 | 0.02 | 0.42 | 0.09 | 0.71 | 0.21 |

### Example 4: Impact of an Increasing Specific Activity of ¹⁷⁷Lu-DOTA-PEG₇-Tz on the Biodistribution of ¹⁷⁷Lu-VHH1 Using a Pre-Targeting Strategy

In this example we describe the effect of specific activity of ¹⁷⁷Lu-DOTA-PEG₇-Tz on the resulting biodistribution of ¹⁷⁷Lu-VHH1 using a pre-targeting strategy. For this, the biodistribution and tumor targeting of ¹⁷⁷Lu-VHH1 using low and high specific activity ¹⁷⁷Lu-DOTA-PEG₇-Tz was evaluated in mice with human FAP expressing tumors. In both cases, mice with small established tumors were first injected with 200 μg (16 nmol) TCO-VHH1 via the tail vein. 4 h post administration of TCO-VHH1, either ±80 μCi or ±500 μCi of ¹⁷⁷Lu-DOTA-PEG₇-Tz was injected via the tail vain. Next, the mice were euthanized by cervical dislocation up to 96 h post injection, dissected after which different organs and tissues were collected. Organs and tissues of interest were weighed and measured for radioactivity using an automatic gamma counter, along with injection standards. Results were expressed as % of injected activity (IA)/g tissue. The obtained update values were used to calculate the respective area under the curve for both conditions.

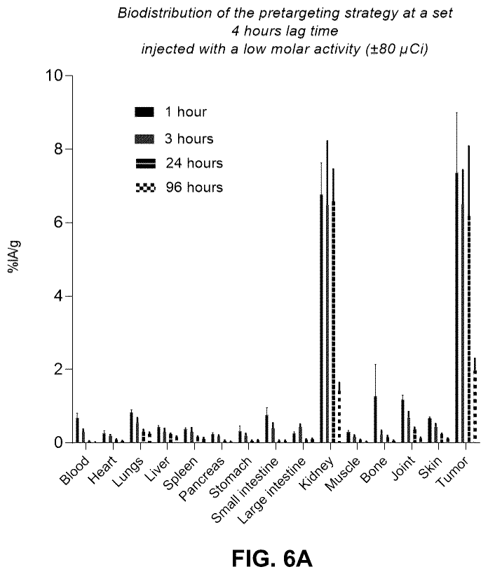

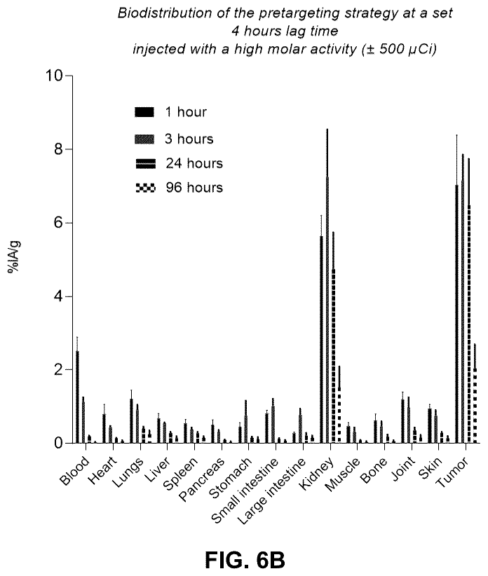

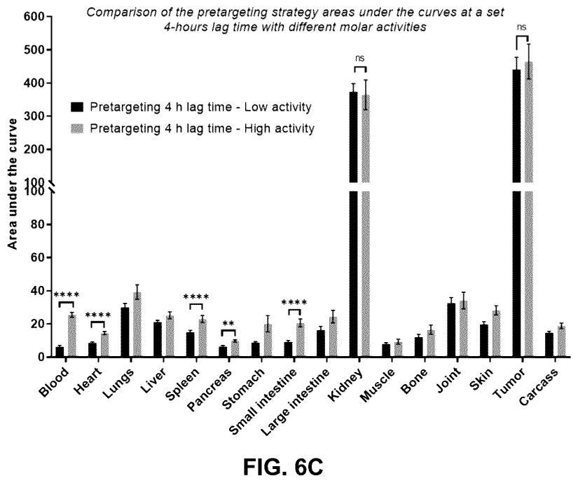

The use of high specific activity ¹⁷⁷Lu-DOTA-PEG₇-Tz resulted in a significant increase of the area under the curve (AUC) in several organs and tissues (P<0.001 for blood, heart, spleen, small intestine and pancreas) as highlighted in Table 16 and FIG. 6.C. However none of these increasing uptake values hamper the use of high specific activity ¹⁷⁷Lu-DOTA-PEG₇-Tz for a pre-targeting strategy. More so, the uptake values and the resulting AUCs in tumor (P=0.706) and kidneys (P=0.850) were very similar for both conditions, enabling therapeutic amounts of ¹⁷⁷Lu-VHH1 (Table 14, 15, 16 and FIG. 6).

TABLE 14
Uptake values in different organs and tissues for ¹⁷⁷Lu-VHH1 using a pre-targeting
strategy up to 96 h post i.v. injection, using a lag time of 4 h and ¹⁷⁷Lu-DOTA-PEG₇-
Tz at a high specific activity of ±50 μCi. Results are expressed as % injected activity
per gram of tissue (% IA/g). Data presented as mean ± SD (n = 5).

| | 1 h | | 3 h | | 24 h | | 96 h | |
|---|---|---|---|---|---|---|---|---|
| Organ/tissue | MEAN | SD | MEAN | SD | MEAN | SD | MEAN | SD |
| Blood | 0.68 | 0.13 | 0.30 | 0.07 | 0.04 | 0.01 | 0.01 | 0.01 |
| Heart | 0.26 | 0.07 | 0.19 | 0.03 | 0.09 | 0.01 | 0.05 | 0.01 |
| Lungs | 0.83 | 0.07 | 0.54 | 0.14 | 0.31 | 0.05 | 0.25 | 0.04 |
| Liver | 0.43 | 0.04 | 0.30 | 0.09 | 0.24 | 0.02 | 0.16 | 0.02 |
| Spleen | 0.37 | 0.04 | 0.30 | 0.11 | 0.16 | 0.02 | 0.11 | 0.03 |
| Pancreas | 0.24 | 0.04 | 0.17 | 0.04 | 0.06 | 0.01 | 0.03 | 0.01 |
| Stomach | 0.32 | 0.14 | 0.20 | 0.05 | 0.06 | 0.01 | 0.07 | 0.01 |
| Small intestine | 0.75 | 0.21 | 0.40 | 0.14 | 0.06 | 0.01 | 0.06 | 0.01 |
| Large intestine | 0.26 | 0.04 | 0.43 | 0.08 | 0.09 | 0.01 | 0.10 | 0.02 |
| Kidney | 6.76 | 0.87 | 6.48 | 1.74 | 6.58 | 0.88 | 1.42 | 0.22 |
| Muscle | 0.30 | 0.04 | 0.17 | 0.04 | 0.08 | 0.01 | 0.03 | 0.01 |
| Bone | 1.26 | 0.88 | 0.22 | 0.12 | 0.15 | 0.05 | 0.05 | 0.02 |
| Joint | 1.17 | 0.13 | 0.69 | 0.16 | 0.37 | 0.05 | 0.12 | 0.03 |
| Skin | 0.67 | 0.04 | 0.44 | 0.08 | 0.24 | 0.02 | 0.12 | 0.01 |
| Tumor | 7.36 | 1.64 | 6.50 | 0.94 | 6.18 | 1.90 | 1.97 | 0.33 |
| Tumor-kidney ratio | 1.08 | 0.25 | 1.05 | 0.26 | 0.94 | 0.27 | 1.41 | 0.29 |

TABLE 15
Uptake values in different organs and tissues for ¹⁷⁷Lu-VHH1 using a pre-targeting
strategy up to 96 h post i.v. injection, using a lag time of 4 h and ¹⁷⁷Lu-DOTA-PEG₇-
Tz at a high specific activity of ±500 μCi. Results are expressed as % injected activity
per gram of tissue (% IA/g). Data presented as mean ± SD (n = 5).

| | 1 h | | 3 h | | 24 h | | 96 h | |
|---|---|---|---|---|---|---|---|---|
| Organ/tissue | MEAN | SD | MEAN | SD | MEAN | SD | MEAN | SD |
| Blood | 2.51 | 0.38 | 1.13 | 0.12 | 0.19 | 0.02 | 0.03 | 0.01 |
| Heart | 0.79 | 0.27 | 0.43 | 0.04 | 0.14 | 0.01 | 0.06 | 0.02 |
| Lungs | 1.21 | 0.24 | 0.90 | 0.15 | 0.40 | 0.05 | 0.25 | 0.10 |
| Liver | 0.68 | 0.13 | 0.55 | 0.02 | 0.28 | 0.03 | 0.14 | 0.05 |
| Spleen | 0.55 | 0.10 | 0.38 | 0.05 | 0.27 | 0.04 | 0.15 | 0.04 |
| Pancreas | 0.50 | 0.14 | 0.31 | 0.05 | 0.09 | 0.01 | 0.04 | 0.01 |
| Stomach | 0.44 | 0.12 | 0.75 | 0.41 | 0.15 | 0.03 | 0.11 | 0.06 |
| Small intestine | 0.81 | 0.09 | 1.01 | 0.20 | 0.12 | 0.02 | 0.07 | 0.02 |
| Large intestine | 0.28 | 0.03 | 0.77 | 0.17 | 0.21 | 0.07 | 0.15 | 0.06 |
| Kidney | 5.64 | 0.56 | 7.25 | 1.29 | 4.74 | 1.00 | 1.52 | 0.57 |
| Muscle | 0.47 | 0.09 | 0.31 | 0.12 | 0.08 | 0.01 | 0.04 | 0.02 |
| Bone | 0.62 | 0.18 | 0.46 | 0.14 | 0.18 | 0.06 | 0.06 | 0.02 |
| Joint | 1.19 | 0.21 | 0.97 | 0.28 | 0.35 | 0.08 | 0.15 | 0.08 |
| Skin | 0.95 | 0.11 | 0.75 | 0.15 | 0.29 | 0.02 | 0.14 | 0.06 |
| Tumor | 7.04 | 1.35 | 7.15 | 0.71 | 6.49 | 1.25 | 2.04 | 0.65 |
| Tumor-kidney ratio | 1.24 | 0.17 | 1.00 | 0.12 | 1.41 | 0.37 | 1.42 | 0.37 |

TABLE 16
The resulting area under the curves (AUCs) for ¹⁷⁷Lu-
VHH1 using a pre-targeting strategy using either high
molecular activity (about 500 μCi) or the low molecular activity
(about 80 μCi) ¹⁷⁷Lu-DOTA-PEG₇-Tz.

| | Pretargeting 4 h lag time | | | |
|---|---|---|---|---|
| | Low molecular activity | | High molecular activity | |
| Organ/tissue | MEAN | SEM | MEAN | SEM |
| Blood | 6.08 | 0.90 | 25.42 | 1.562 |
| Heart | 8.47 | 0.63 | 14.41 | 0.9539 |
| Lungs | 29.96 | 2.37 | 39.16 | 4.363 |
| Liver | 21.12 | 1.04 | 25.07 | 2.137 |
| Spleen | 14.91 | 1.13 | 22.88 | 2.147 |
| Pancreas | 6.23 | 0.71 | 9.69 | 0.7536 |
| Stomach | 8.725 | 0.58 | 20 | 4.965 |
| Small intestine | 9.13 | 0.94 | 20.53 | 2.354 |
| Large intestine | 16.23 | 2.29 | 24.3 | 3.843 |
| Kidney | 373.10 | 25.09 | 364.1 | 44.86 |
| Muscle | 7.87 | 0.80 | 9.195 | 1.506 |
| Bone | 12.04 | 1.57 | 16.44 | 2.79 |
| Joint | 32.48 | 3.38 | 34.02 | 5.11 |
| Skin | 19.64 | 1.66 | 28.1 | 2.78 |
| Tumor | 440.70 | 37.07 | 464.5 | 52.94 |

### Example 5: Therapeutic Potential of Pre-Targeted ²²⁵Ac-VHH1

This example describes the therapeutic potential of pre-targeted ²²⁵Ac-VHH₁, which results from the in vivo coupling of TCO-VHH1 and ²²⁵Ac-DOTA-PEG₇-Tz using a pre-targeting strategy, which was assessed by measuring its capacity to inhibit tumor growth in FAP-expressing human glioblastoma tumor (U87 MG) xenografted mice. Mice with small established tumors (50-80 mm³) were treated six consecutive times over a period of three weeks. The two groups where the pre-targeting strategy was used, were first i.v. injected with 200 μg (16 nmol) TCO-VHH1 followed by either (i) a high (2.70 μCi; ±3.5 μg) or (ii) low (0.54 μCi; ±3.5 μg) dose of ²²⁵Ac-DOTA-PEG₇-Tz. The lag time between the first (TCO-VHH1) and the second injection (²²⁵Ac-DOTA-PEG₇-Tz) was 8 h. The obtained mean survival of mice i.v. injected with the pre-targeting strategy were compared with the mean survival of mice i.v. injected with either (i) direct labeled ²²⁵Ac-DOTA-VHH1 (0.54 μCi; ±5 μg), or with (ii) direct labeled irrelevant ²²⁵Ac-DOTA-R3B23 (0.54 μCi; ±5 μg). A last group only received 200 μg (16 nmol) TCO-VHH1. Tumor volume and animal weight were measured repeatedly. Dropouts were considered when one of the following endpoints was reached: for subcutaneous tumors (i) tumor size of >1500 mm³, (ii) >20% weight loss or (iii) the presence of necrotic tumor tissue.

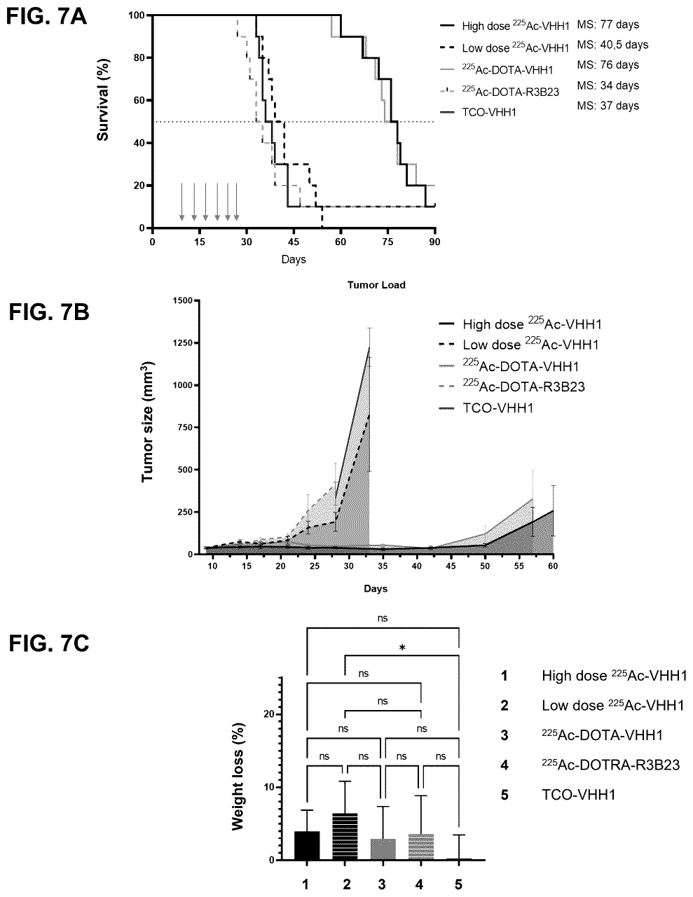

Mice treated with the high dose of pre-targeted ²²⁵Ac-VHH1 could effectively reduce the tumor growth (FIG. 7B) and lived significantly longer compared to mice treated with ²²⁵Ac-DOTA-R3B23 (p=0.01, Log-rank Mantel-cox test) or vehicle solution (p=0.01, Log-rank Mantel-cox test) (FIG. 7A). Moreover, no significant difference in weight loss between these groups was observed (FIG. 7C), indicating that no acute toxicity signs were present after the injection of the different activities of ²²⁵Ac-VHH1. A treatment with high radioactive level (high dose) of pre-targeted ²²⁵Ac-VHH1 was more effective compared to a low radioactive level (low dose) of pre-targeted ²²⁵Ac-VHH1 (p<0.0001, Log-rank Mantel-cox test) as depicted in FIGS. 7A and 7B. Indeed, mice injected with direct labeled ²²⁵Ac-DOTA-VHH1 lived significantly longer compared to mice injected with the low dose pre-targeted ²²⁵Ac-VHH1, suggesting pre-targeting requires higher levels of administered radioactivity to obtain the same therapeutic effect as for the direct labeling strategy.

In conclusion, pre-targeted ²²⁵Ac-VHH1 is effective in FAP+ expressing tumor xenografted mouse models without any relevant signs of acute toxicity.

### Example 6: Long-Term Toxicology Follow-Up of Healthy Female C57BI/6 Mice Treated with Pre-Targeted ²²⁵Ac-VHH1

This example describes the long-term toxicology follow-up of healthy female C57BI/6 mice treated with pre-targeted ²²⁵Ac-VHH1. The health status of treated mice is assessed during six months, after which they are sacrificed and processed for full histopathological analysis. To this, healthy female C57BI/6 mice were i.v. injected with pre-targeted ²²⁵Ac-VHH1 and direct labeled ²²⁵Ac-DOTA-VHH1, via six consecutive injections on day 0, 4, 7, 11, 14 and 18. The groups receiving pre-targeted compounds were first i.v. injected with 200 μg (16 nmol) TCO-VHH1 followed by either (i) a high radioactive dose (2.70 μCi; ±3.5 μg) or (ii) a low radioactive dose (0.54 μCi; ±3.5 μg) of ²²⁵Ac-DOTA-PEG₇-Tz. The lag time between the first (TCO-VHH1) and the second injection (²²⁵Ac-DOTA-PEG₇-Tz) was 8 h. The obtained mean survival and the weight loss of mice i.v. injected with the pre-targeted compounds were compared with the mean survival and the weight loss of mice i.v. injected with either (i) direct labeled ²²⁵Ac-DOTA-VHH1 (0.54 μCi; ±5 μg), or with (ii) direct labeled irrelevant ²²⁵Ac-DOTA-R3B23 (0.54 μCi; ±5 μg). A last group only received 200 μg (16 nmol) TCO-VHH1. Study dropouts were considered when one of the following endpoints was reached: (i) a sudden >20% weight loss, (ii) a body condition score of >5.

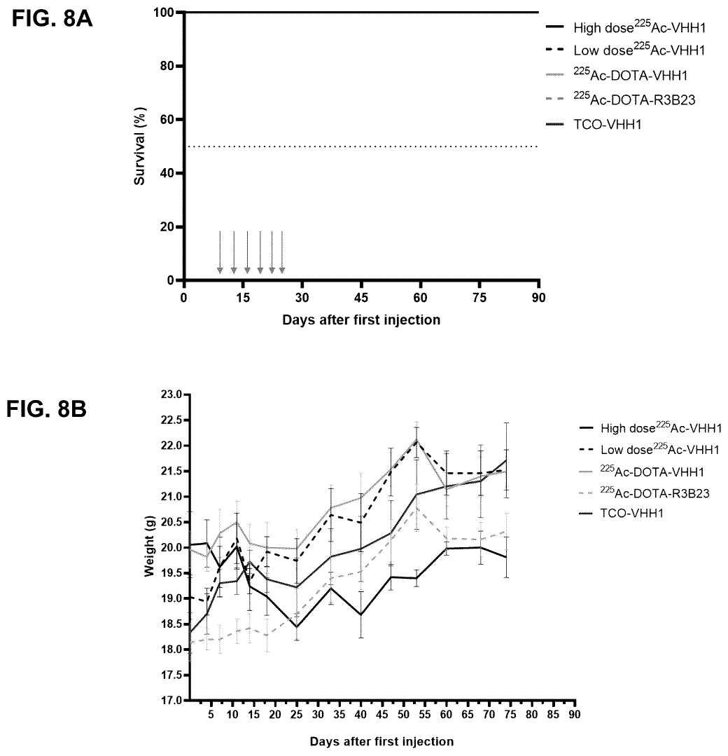

To date, no significant difference in survival (FIG. 8A) or significant drop in weight has been observed between the different experimental groups. The average weight of the mice receiving high dose pre-targeted ²²⁵Ac-VHH1 seems slightly lower compared to the other groups, however not significantly lower (FIG. 8B). Histopathological analysis will be performed after 6 months.

In conclusion, an ongoing toxicity assessment using pre-targeted ²²⁵Ac-VHH1 did not indicate significant impact on weight progression until three months after treatment initiation, indicating that pre-targeted ²²⁵Ac-VHH1 appears to be safe, it does not lead to acute signs of toxicity, and allows the administration of high radioactive amounts of ²²⁵Ac-labeled radiopharmaceuticals.

### Example 7: Minimal VHH Dissociation Rate Constant (k_{off}) Determination for Click Reaction

This example describes the comparison of different TCO conjugated VHHs (TCO-VHHs) binding the same target with a different dissociation rate constant (K_{off}) and the influence of that dissociation rate constant on the click reaction with tetrazine.

Five VHHs binding the target (human FAP) with different dissociation rate constants were conjugated to TCO-NHS. Using the BLI technology on Octet system the binding kinetics towards human FAP were determined for each of the five VHHs pre- and post-TCO conjugation. The table below shows the results of this experiment, from which we concluded that the dissociation rate constant prior and post TCO conjugation did not significantly change and that the presence of the TCO group on the VHH did not significantly alter its binding kinetics.

TABLE 17

| | Pre conjugation | | | Post conjugation | | |
|---|---|---|---|---|---|---|
| VHH | kₒₙ (1/Ms) | k_{off} (1/s) | K_{D} (M) | kₒₙ (1/Ms) | k_{off} (1/s) | K_{D} (M) |
| VHH1 | 1.77E+06 | 8.52E−05 | 4.82E−11 | 6.94E+05 | 4.99E−05 | 7.19E−11 |
| VHH6 | 3.41E+06 | 1.88E−04 | 5.51E−11 | 1.07E+06 | 1.57E−04 | 1.47E−10 |
| VHH7 | 2.40E+06 | 1.97E−04 | 8.19E−11 | 7.46E+05 | 1.23E−04 | 1.66E−10 |
| VHH8 | 1.01E+06 | 6.79E−04 | 6.72E−10 | 8.33E+05 | 5.59E−04 | 6.71E−10 |
| VHH9 | 3.98E+06 | 1.80E−03 | 4.53E−10 | 1.26E+06 | 1.20E−03 | 9.51E−10 |

Next, Octet Streptavidin (SA) Biosensors were coated with 5 μg/mL biotinylated human FAP in assay buffer (HBS+0.5% BSA+0.1% Tween20) for 5 minutes. The sensors were then dipped in wells containing a 5 nM solution of the different VHHs (both TCO conjugated and unconjugated as control) for 20 minutes (association step) and further transferred to the assay buffer (dissociation step) for 30 minutes. After the dissociation phase, that simulates the lag time, the sensors were transferred to either a well containing 50 nM of a tetrazine-coupled, non-FAP binding R3B23 (Tz-R3B23) or to a well containing assay buffer as a control. This reaction was allowed to proceed for 20 minutes (experiment 1) or 30 minutes (experiment 2) after which the sensors were washed for 30 minutes in assay buffer. In the second experiment the TCO-VHHs were measured in duplicate (referred to as assay 1 and 2 of ‘experiment 2’ in the table below). Using the epitope binning function of ‘Octet Analysis Software’ we then determined the difference in signal between the VHHs incubated with Tz-R3B23 and the VHHs incubated with assay buffer. This value is referred to as the span and is displayed in the table below. The span of unconjugated VHHs was considered as noise. When a TCO-VHH demonstrated a span value higher than the one of noise, we concluded that a click reaction took place.

TABLE 18

| | | Experiment 2 | Experiment 2 |
|---|---|---|---|
| | Experiment 1 | 1^{st} assay | 2^{nd} assay |
| VHH | Span | Span | Span |
| VHH1 | 0.0089 | 0.0004 | |
| VHH6 | 0.0021 | 0.0017 | |
| VHH7 | 0.0082 | 0.0018 | |
| VHH8 | 0.0096 | 0.0028 | |
| VHH9 | 0.0040 | −0.0008 | |
| TCO-VHH1 | 0.0237 | 0.0334 | 0.0257 |
| TCO-VHH6 | 0.0230 | 0.0287 | 0.0229 |
| TCO-VHH7 | 0.0145 | 0.0186 | 0.0230 |
| TCO-VHH8 | 0.0224 | 0.0218 | 0.0341 |
| TCO-VHH9 | 0.0015 | 0.0091 | 0.0113 |

In both experiments the span of unconjugated VHH8 (indicated in bold in the table above) was the highest and determined the noise value. In ‘experiment 1’ TCO-VHH9 was the only value below the noise value, indicating that no click reaction took place for TCO-VHH9, while it did take place for the other 4 VHHs. In ‘experiment 2’ TCO-VHH9 showed a span just above the noise value in both assays, but this span was by far the lowest detected in the assay, indicating again that TCO-VHH9 had the worst click reaction of all analyzed VHHs.

From this data we concluded that pre-targeting of the human FAP target with TCO-VHH preferably involves a VHH that binds to its antigen with a dissociation constant of 104 or lower.

### Example 8: Pre-Targeting on hHER2 and hFOLR1

This example describes the click reaction of a tetrazine conjugated molecule on TCO-conjugated VHHs targeting human HER2 (hHER2) or human FOLR1 (hFOLR1).

Two different VHHs were conjugated to NHS-TCO: VHH2 binding hFOLR1 and VHH4 binding hHER2. Using Surface Plasmon Resonance (Biacore) we determined the binding kinetics of the unconjugated and TCO-conjugated VHH (TCO-VHH) for the respective target as described earlier for VHH2 and VHH8 (EP3718574A1). In case the binding kinetics of the VHH for its target would have been drastically reduced after TCO conjugation, the VHH would not have been appropriate for further pre-targeting experiments.

Table 19 shows that the binding kinetics of the VHH2 to hFOLR1 and VHH4 to hHER2 were barely affected after TCO conjugation. This allowed us to continue with the further exploration of these VHHs and their targets for use in pre-targeting applications.

TABLE 19
binding kinetics of the VHHs before and after
TCO conjugation for their appropriate target.

| | Pre conjugation | | | Post conjugation | | |
|---|---|---|---|---|---|---|
| VHH | kₒₙ (1/Ms) | k_{off} (1/s) | K_{D} (M) | kₒₙ (1/Ms) | k_{off} (1/s) | K_{D} (M) |
| VHH2 | 3.54E+06 | 1.67E−04 | 4.70E−11 | 2.61E+06 | 1.92E−04 | 7.34E−11 |
| VHH4 | 2.22E+05 | 4.47E−04 | 2.01E−09 | 1.05E+05 | 7.91E−04 | 7.57E−09 |

Next, the click reaction was verified using SPR. 5 μg/mL recombinant hHER2 in 10 mM NaOAc pH 4.5 or 5 μg/mL recombinant hFOLR1 in 10 mM NaOAc pH 5.5 was immobilized on a Biacore Sensor Chip CM5 to a target level of 740 Response Units (RU) and 250 RU, respectively. To verify the click reaction, an association phase was started with 62.5 nM TCO-VHH4 or unconjugated VHH4 for 180 s followed by a dissociation phase for 600 s. For the VHH7 binding FOLR1 a VHH concentration of 5 nM was used, and the association and dissociation phases took 450 s and 900 s, respectively. To induce the click reaction, Tz coupled R3B23 (Tz-R3B23; R3B23 does not bind hHER2 or hFOLR1) was injected over the flow cell for 350 seconds at a 100 times molar excess (6250 nM or 500 nM) followed by a dissociation step. As a negative control for the click reaction, we injected assay buffer (HBS) instead of the Tz-R3B23 over the flow cell. Using the Biacore evaluation software we determined the difference between the relative response before and after the click reaction with either the Tz-R3B23 or the negative control assay buffer. This difference is further on referred to as span. Table 20 shows the obtained results.

TABLE 20
Span of the Relative Response on Biacore upon administration
of a tetrazine conjugated click ligand on TCO-VHHs and
unconjugated VHHs bound on their respective target.

| | TCO-VHH | Unconjugated VHH |
|---|---|---|
| VHH | Span | Span |
| VHH2 | 12.3 RU | 0.6 RU |
| VHH4 | 17.1 RU | 1.0 RU |

In both experiments the span of TCO-VHH was at least tenfold higher compared to the unconjugated VHH, indicating that the click reaction only took place when TCO-VHHs are bound on their target, while it did not take place when unconjugated VHH bound on the target.

From this data we concluded that a TCO-VHH, when bound to either hHER2 or hFOLR1, can undergo a click reaction specific to a tetrazine conjugated molecule. This allowed us to expand the scope of our pre-targeting work to hHER2 and hFOLR1 as targets.

1. A method of treating a patient in need thereof, said method comprising: administration of a heavy chain variable domain derived from a heavy chain antibody (VHH), or a fragment thereof, said VHH or said fragment comprising a click group, wherein said click group has not underwent a click reaction upon administration of said VHH or said fragment thereof to said patient.

2. A heavy chain variable domain derived from a heavy chain antibody (VHH), or a fragment thereof, comprising a click group, wherein said VHH, or said fragment thereof, is able to specifically bind an antigen with a dissociation constant (k_{off}) equal to or smaller than 10⁻² s⁻¹.

3. The VHH, or a fragment thereof, of claim 2, wherein said click group is an alkene, an alkyne, an azide, a nitrone.

4. The VHH, or a fragment thereof, of claim 2, wherein said antigen is present on the surface of a tumour cell or in a tumour microenvironment.

5. The VHH, or a fragment thereof, of claim 2, wherein said antigen is human fibroblast activation protein (FAP), human folate receptor alpha (FOLR1), or human epidermal growth factor receptor 2 (HER2).

6. A method of treatment and/or diagnosis of a patient with cancer comprising the administration of a combination, said combination comprising:
a heavy chain variable domain derived from a heavy chain antibody (VHH), or a fragment thereof, comprising a click group, as defined in claim 2; and
a labelled compound, comprising a label and a click group;
wherein said VHH, or fragment thereof, is able to specifically bind an antigen associated with said cancer with a dissociation constant (k_{off}) equal to or smaller than 10⁻² s⁻¹;
wherein said method comprises a first administration of said VHH, or fragment thereof, comprising a click group, and a subsequent administration of said labelled compound to said patient in need thereof;
wherein said click group comprised in said VHH, or fragment thereof, forms a click pair with said click group comprised in said labelled compound.

7. The method of claim 6, wherein said click group comprised in said labelled compound is an alkene, an alkyne, an azide, a nitrone, or a tetrazine.

8. The method of claim 6, wherein said labelled compound can be represented by CL-L¹-L²-R*, wherein CL is said click group, L¹ is a first linker or a bond, L² is a second linker, and R* is said label.

9. The method of claim 8, wherein L¹ is PEGₙ, wherein n is an integer from 1 up to 10.

10. The method of claim 8, wherein L² is a chelator or a benzoate linker.

11. The method of claim 6, wherein said label is a radionuclide.

12. The method of claim 11, wherein said radionuclide is an α-emitting radionuclide or a β-emitting radionuclide.

13. The method of claim 11, wherein said radionuclide is a positron-emitting radioisotope (PET) or a γ-emitting radioisotopes (SPECT).

14. The method of claim 6, wherein said labelled compound can be represented by ¹⁷⁷Lu-DOTA-PEG₇-Tz, wherein Tz is a tetrazine.

15. The method of claim 6, wherein no blood clearing agent is administered to said subject between said first and subsequent administration.

16. The method of claim 11, wherein the time between said first and subsequent administration is at least 2 hours.

17. The method of treatment of claim 1, wherein said patient has cancer.

18. The method of treatment of claim 17, wherein said cancer is a solid cancer or said patient is diagnosed with a solid cancer.

19. The VHH, or a fragment thereof, of claim 3, wherein said alkene is a trans-cyclooctene.

20. The VHH, or a fragment thereof, of claim 3, wherein said alkyne is BCN, BARAC or DIBAC.

21. The method of claim 7, wherein said alkene is a trans-cyclooctene or said alkyne is BCN, BARAC or DIBAC.

22. The method of claim 7, wherein said alkene is a tetrazine.

23. The method of claim 10, wherein said chelator is 1,4,7,10-tetraazacyclododecane-1,4,7,10-tetraacetic acid (DOTA), 1,4,7-triazacyclononane-1,4,7-triacetic acid (NOTA), N,N′-bis[(6-carboxy-2-pyridil)methyl]-4,13-diaza-18-crown-6 (Macropa), 2,2′,2″,2′-(1,10-dioxa-4,7,13,16-tetraazacyclooctadecane-4,7,13,16-tetrayl)tetraacetic acid (Crown) or 3,6,9,15-tetraazabicyclo[9.3.1]pentadeca-1(15), 11,13-triene-3,6,9-triacetic acid (PCTA).

24. The method of claim 10, wherein said benzoate linker is guadinomethyl benzoate (GMIB).

25. The method of claim 12, wherein said radionuclide is actinium-225, astatine-211, bismuth-212, bismuth-213, caesium-137, chromium-51, cobalt-60, copper-67, dysprosium-165, erbium-169, fermium-255, gold-198, holium-166, iodine-125, iodine-131, iridium-192, iron-59, lead-212, lutetium-177, molydenum-99, palladium-103, phosphorus-32, potassium-42, rhenium-186, rhenium-188, samarium-153, radium-223, radium-224, ruthenium-106, sodium-24, strontium-89, scandium-47, terbium-149, terbium-161, thorium-227, xenon-133, ytterbium-169, ytterbium-177, or yttrium-90.

26. The method of claim 25, wherein said radionuclide is lutetium-177, terbium-161 or iodine-131.

27. The method of claim 13, wherein said radionuclide is iodine-131, yttrium-90, iodine-125, lutetium-177, rhenium-186, rhenium-188, terbium-161, terbium-149, technetium-99m, indium-111, xenon-133, thallium-201, fluorine-18, gallium-68, gallium-67, copper-67, scandium-44, scandium-43, iodine-123, iodine-124, zirconium-89, or copper-64.
